(19) United States
(12) Patent Application Publication
(10) Pub. No.: US 2002/0006967 A1
(43) Pub. Date: Jan. 17, 2002

(54) METHODS OF TREATMENT OF OCULAR NEOVASCULARIZATION

(76) Inventor: PETER A. CAMPOCHIARO, BALTIMORE, MD (US)

Correspondence Address: Dike, Bronstein, Roberts & Cushman, Intellectual Property Practice Group, EDWARDS & ANGELL, P. O. Box 9169, BOSTON, MA, 02209, US

(21) Appl. No.: 09/100,790

(22) Filed: Jun. 18, 1998

Related U.S. Application Data
(60) Provisional application No. 60/050,225, filed on Jun. 19, 1997.

(51) Int. Cl. C07C 229/00; A61K 31/075; C07D 233/54; A61K 31/47; C07D 233/60; C07D 233/90

(52) U.S. Cl. 514/715; 514/716; 514/717; 560/147; 560/155; 548/338.1

(57) ABSTRACT

The present invention includes methods for treatment and prophylaxis of eye disorders and injuries, particularly treatment and prophylaxis of ocular neovascularization and disorders, especially a vasculopathy that affects retinal or chorodial vessels. The methods of the invention in general comprise administration of a therapeutically effective amount of a compound that inhibits farnesyl-protein transferase to a subject suffering from or susceptible to ocular neovascularization or associated disorder.

The present application is a continuation of U.S. provisional application serial number 60/050,225, filed Jun. 19, 1997, incorporated herein by reference.

### BACKGROUND

1. Field of the Invention

The present invention relates to methods for treatment of ocular neovascularization and, more particularly, to use of one or more farnesyl-protein transferase inhibitor compounds to treat a subject suffering from or susceptible to ocular neovascularization or a disorder associated therewith.

2. Background

New blood vessel formation or neovascularization occurs in a number of ocular disease processes. In particular, retinal neovascularization can result in occlusion of normal retinal blood vessels which leads to retinal ischemia. These disorders have been collectively referred to as ischemic retinopathies and include proliferative diabetic retinopathy (PDR), retinopathy of prematurity, central and branch retinal vein occlusion, and other systematic vasculopathies that affect retinal vessels. The most common affliction is diabetic retinopathy, a major cause of new blindness in developed countries (H. Kahn, Am. J. Ophthalmol., 78:58-67 (1974)).

Panretinal laser photocoagulation can improve retinal oxygenation and has caused regression of retinal neovascularization in certain cases. See C. Pomaras et al., Ophthalmology, 97:1329-1333 (1990). In many instances however, laser photocoagulation is not effective. For example, laser photocoagulation can not be delivered to a retina obscured by vitreous hemorrhage. For patients with severe neovascularization, even with clear media, laser treatment alone may not prevent vision loss. See S. deBustros et al., Arch. Ophthalmol., 105:196-199 (1987); H. Flynn et al., Ophthalmology, 97:1329-1333 (1990).

Choroidal neovascularization occurs in diseases in which there are abnormalities in Bruch's membrane and the surrounding tissues. The most common disease of this type is age-related macular degeneration, a major cause of visual loss in patients over the age of 60 (Macular Photocoagulation Study Group, Arch. Ophthalmol., 109:1109 (1991)). It is estimated that by the year 2000 there will be two million patients in the United States with age-related macular degeneration. Currently, no effective treatment exists for choroidal neovascularization due to age-related macular degeneration.

The oncogene protein Ras is one of several GTP-binding proteins that are modified by a prenyl group. Farnesyl has been reported to modify Ras, Rab, Rho and other small GTP-binding proteins, and geranylgeranyl has been reported to modify Rab, Rho and other small GTP-binding proteins. A. Garcia et al., J. Biol. Chem., 268:18415-18418 (1993).

Farnesyl-protein transferase (FTase), the enzyme that catalyzes the lipid modification of Ras, has become a target for anticancer therapies. Inhibition of FTase has been reported to block the growth of Ras-transformed cells in soft agar. It also has been reported that certain inhibitors of FTase block the processing of the Ras oncoprotein intracellularly. N. E. Kohl et al., Science, 260:1934-1937 (1993); G. L. James et al., Science, 260:1937-1942 (1993); N. E. Kohl et al., Proc. Natl. Acad. Sci U.S.A., 91:9141-9145 (1994); and N. E. Kohl et al., Nature Medicine, 1:792-797 (1995). See U.S. Pat. Nos. 5,238,922; 5,571,792; and 5,571,835; WO 94/10138; WO 94/04561; WO 94/10138; WO 96/21456; and WO 97/02817, which report certain compounds that inhibit farnesyl-protein transferase.

### SUMMARY OF THE INVENTION

The present invention includes methods for treatment and prevention of eye disorders and injuries, particularly treatment and prevention of ocular neovascularization and disorders associated therewith such as diabetic retinopathy, retinopathy of prematurity, retinal vein and artery occlusion, age-related macular degeneration, corneal graft rejection, neovascular glaucoma, retrolental fibroplasia, Eales disease, and other vasculopathies that affect retinal or chordial vessels.

The methods of the invention in general comprise administration of a therapeutically effective amount of a compound that inhibits famesyl-protein transferase (a FTase inhibitor) to a patient in need of treatment, such as a mammal suffering from or susceptible to ocular neovascularization and disorders associated therewith.

A wide variety of FTase inhibitor compounds can be employed in the methods of the invention. For example, suitable compounds have been reported previously including those in U.S. Pat. Nos. 5,238,922; 5,571,792; and 5,571,835; WO 94/10138; WO 94/04561; WO 94/10138; WO 96/21456; and WO 97/02817.

Particularly preferred FTase inhibitor compounds for use in the methods of the invention exhibit good activity in a standard in vitro FTase inhibition assay, preferably an IC₅₀ (concentration required to inhibit FTase activity by 50% relative to control) in such an assay of about 100 nM or less, more preferably an IC₅₀ about 50 nM or less. A standard assay includes the following steps a) through c):

a) admixing in a suitable assay solution 1) a potential FTase inhibitor compound, 2) [³H]farnesyl diphosphate, 3) famesyl-protein transferase and 4) H-Ras;

b) incubating the test mixture for 15 minutes at 37° C.; and

c) measuring utilization of [³H]farnesyl diphosphate over that time relative to a control mixture that is prepared and incubated under the same conditions as the assay mixture but does not include the potential inhibitor compound. A suitable assay solution includes 50 mM HEPES, pH 7.5, 5 mM MgCl₂, 5 mM dithiothreitol. References herein to a standard in vitro farnesyl-protein transferase inhibition assay are intended to refer to that protocol. That protocol also has been described in A.M. Garica et al., J. Biol. Chem., 268:18415-18418 (1993).

Even more preferred are FTase inhibitor compounds that exhibit good activity as defined above, and are also selective for FTase inhibition, i.e. the compounds are relatively poor inhibitors of other prenyl-protein transferases, particularly geranylgeranyl-protein transferase I. Geranylgeranyl protein-transferase I is related to famesyl-protein transferase and catalyzes the prenylation of certain GTP-binding proteins as discussed above. The number of geranylgeranylated proteins in a cell significantly exceeds the number of farnesylated proteins, and therefore preferred compounds for use in the methods of the invention are selective for inhibition of famesylation to avoid undesired pharmacological effects that could arise from inhibition of geranylgeranyl-protein transferase I.

More specifically, preferred FTase inhibitor compounds exhibit at least about a 20-fold greater inhibition of famesyl-protein transferase relative to inhibition of geranylgeranyl-protein transferase I as measured in standard in vitro famesyl-protein transferase and geranylgeranyl-protein transferase I inhibition assays, more preferably at least about a 30-fold greater inhibition of famesyl-protein transferase relative to inhibition of geranylgeranyl-protein transferase I.

Preferred selective FTase inhibitor compounds also may exhibit an IC₅₀ (concentration required to inhibit geranylgeranyl-protein transferase I activity by 50% relative to control) of about 200 nM or greater in a standard in vitro geranylgeranyl-protein transferase I inhibition assay, more preferably an IC₅₀ of about 500 nM or greater. A standard in vitro geranylgeranyl-protein transferase I inhibition assay includes the following steps a) through c):

a) admixing in a suitable assay solution 1) a potential inhibitor compound, 2) [³H]geranylgeranyl diphosphate, 3) geranylgeranyl-protein transferase I, and 4) H-Ras;

b) incubating the test mixture for 15 minutes at 37° C.; and

c) measuring utilization of [³H]geranylgeranyl diphosphate over that time relative to control mixture that is the prepared and incubated under the same conditions as the assay mixture but does not include the potential inhibitor compound. A suitable assay solution includes 50 mM HEPES, pH 7.5, 5 mM MgCl₂, 5 mM dithiothreitol. References herein to a standard in vitro geranylgeranyl-protein transferase I inhibition assay are intended to refer that protocol. That protocol also has been described in A. M. Garica et al., J. Biol. Chem., 268:18415-18418 (1993).

Generally preferred for use in the methods are FTase inhibitor compounds that are competitive with protein substrates for famesyl-protein transferase. Such inhibitors may contain a thiol moiety, although substrate-competitive inhibitors are known that do not contain a thiol group. Some of those compounds are cysteine-containing molecules related in some respect to the “CAAX” motif that is the signal for protein prenylation. That CAAX motif has been defined as C=Cys, A=any aliphatic amino acid, X=any amino acid (N. Kohl et al., Nature Medicine, 1:791 (1995)). Such preferred inhibitor compounds are exemplified by compounds of groups (a) through (z) as those groups are specified below.

Also suitable for use in the methods of the invention are FTase inhibitors that are competitive with farnesyl pyrophosphate. These compounds may contain e.g. a phosphate functionality, or be free of phosphorous groups, e.g. such as in chaetomellic acids. These compounds are exemplified by compounds of groups (aa) through (gg), as those groups are specified below. Further suitable for use in the methods of the invention are FTase inhibitors that comprise features of both classes of inhibitors, i.e. bi-substrate compounds that are competitive with protein substrates and with farnesyl pyrophosphate for FTase.

Specifically preferred FTase inhibitor compounds for use in the methods of the invention include the following where the compound is structurally depicted above the chemical name thereof, and pharmaceutically acceptable salts of the compounds.

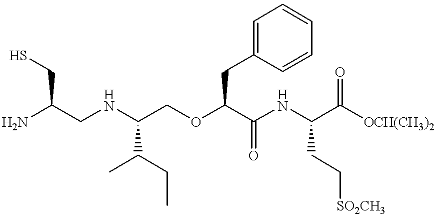

(2(S)-[2(S)-[2(R)-amino-3-mercapto]-propylamino-3(S)-methyl]pentyloxy-3-henylpropionyl-methionine-sulphone isopropyl ester).

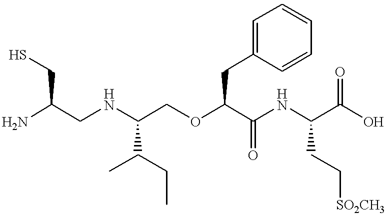

(2(S)-[2(S)-[2(R)-amino-3-mercapto]-propylamino-3(S)-methyl]pentyloxy-3-phenylpropionyl-methionine-sulphone).

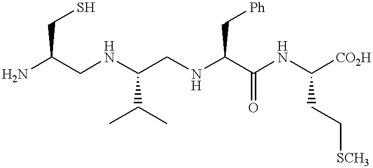

(N-[2(S)-[2(R)-amino-3-mercaptopropylamino]-3-methylbutyl]-L-phenylalaninyl-L-methionine).

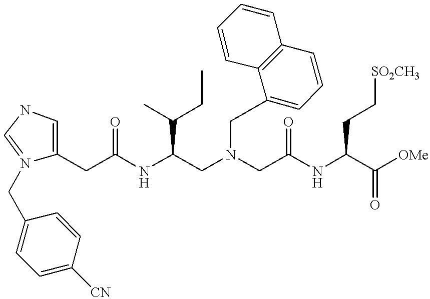

(N-[2(S)-N′-(1-(4-cyanophenylrnethyl)-1 H-imidazol-5-yl-acetyl)amino-3(S)-methylpentyl]-N-l-naphthylmethyl-glycl-methionine-sulphone methylester).

Other aspects of the invention are disclosed infra.

### DETAILED DESCRIPTION OF THE INVENTION

As stated above, the invention provides new therapeutic methods for treatment and prevention of eye disorders and injuries, particularly treatment and prevention of ocular neovascularization and associated disorders. The methods of the invention in general comprise administration of a therapeutically effective amount of a farnesyl-protein transferase inhibitor compound to a patient in need of such treatment.

The efficacy of any particular farnesyl-protein transferase inhibitors in the therapeutic methods of the invention can be readily determined. For example, compounds with superior intrinsic inhibitory activity against and selectivity for farnesyl-protein transferase can be identified through the in vitro assays discussed above.

In vivo assays will be useful for the subsequent evaluation of potent FTase inhibitors for use in treatment of ocular neovascularization. A mouse oxygen-induced ischemic retinopathy model is a preferred assay. A suitable protocol provides that seven days after birth mice are placed in a high oxygen environment which inhibits the development of the normal retinal vessels. After 5 days in that oxygen environment, the mice are transferred to the relative hypoxia of room air where the retina becomes ischemic and retinal neovascularization occurs in 100% of the animals. The amount of neovascularization can be suitably quantitatively determined using a selective endothelial cell marker and image analysis.

For an initial in vivo evaluation of a FTase inhibitor compound, the maximum tolerated dose of the inhibitor compound is given subcutaneously twice a day. Dosing begins as soon as the animals are removed from the high oxygen environment on post natal day 12. The mice are treated for five days with drug or vehicle control alone and then sacrificed. The eyes are frozen in optimal cutting temperature embedding compound (Miles, Elkhart, Ind.) and then 10 μm sections cut and every tenth section stained with an endothelial cell specific lectin. The endothelial cell area on the surface of the retina is suitably measured using a 3 CCD camera, a frame grabber, and Image Pro Plus software. This general protocol has been previously employed for evaluation of αᵥβ₃ integrin inhibitors. J. Luna et al., Lab. Invest., 75:563-573 (1996).

A second in vivo assay for evaluating efficacy of FTase inhibitor compounds in therapeutic methods of the invention involves overexpression of vascular endothelial growth factor (VEGF) in the photoreceptors resulting in focal intraretinal and subretinal neovascularization. Neovascularization can be quantitatively determined by perfusing animals with fluorescein-labeled dextran and then preparing retinal whole mounts. The neovascularization is quantitated by fluorescence microscopy and image analysis. It has been found that the transgene is turned on at one week after birth, and at three weeks after birth 100% of animals have subretinal neovascularization, the area of which varies by less than 5% from animal to animal.

In this assay, dosing suitably begins on postnatal day 7 and will continue for two weeks. Control animals are treated with vehicle alone. On post natal day 21 the animals are perfused through the left ventricle with fluorescein labeled dextran and then the eyes are removed, fixed in 10% phosphate-buffered formalin, and the retinas dissected and whole mounted. The retinas are viewed with fluorescence microscopy and neovascularization in the subretinal space is quantitated, e.g. using Image ProPlus software.

In addition to the above discussed preferred FTase inhibitors, suitable FTase inhibitors compounds for use in the methods of the invention are disclosed below (including those compounds of groups (a) through (gg) as those groups of compounds are defined below, and other compounds defined below). It should be appreciated however that the present invention is not limited by the particular FTase inhibitor, and the invention is applicable to any such FTase inhibitor compound now known or subsequently discovered or developed.

FTase inhibitor compounds suitable for use in the methods of the invention will include those compounds that incorporate a cysteinyl or sulfhydryl containing moiety at the N-terminus of the molecule. More specifically, the following compounds will be useful in the methods of the invention:

(a) a peptide that comprises the amino acids CA₁A₂X, wherein:

C=cysteine;

A₁=an aliphatic amino acid;

A₂=an aliphatic amino acid; and

X=any amino acid;

(b) Cys-Xaa¹-Xaa²-Xaa³-NRR¹, wherein Cys=cysteine;

Xaa¹=any amino acid in the natural L-isomer form;

Xaa²=any amino acid in the natural L-isomer form; and

Xaa³=NRR¹=an amide of any amino acid in the natural L isomer form,

wherein R and R¹ are independently selected from hydrogen, C₁-C₁₂ alkyl, aralkyl, or unsubstituted or substituted aryl;

(c) Cys-Xaa¹-Xaa²-Xaa³, wherein Cys=cysteine;

Xaa¹=any amino acid;

Xaa²=the amino acid phenyl alanine or a p-fluorophenylalanine; and

Xaa³=any amino acid;

(d) Cys-Xaa¹-dXaa²-Xaa³, wherein

Cys=cysteine;

Xaa¹=any amino acid in the natural L-isomer form;

dXaa²=any amino acid in the natural L-isomer form; and

Xaa³=any amino acid in the natural L-isomer form;

(e) compounds of the following formula, which compounds are also disclosed in U.S. Pat. No. 5,238,922, incorporated herein by reference,

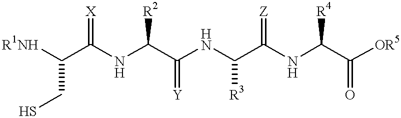

wherein:

X, Y, and Z are independently H₂ or O, provided that at least one of these is H₂;

R¹ is H, an alkyl group, an acyl group, an alkylsulfonyl group or aryl sulfonyl group, wherein alkyl and acyl groups comprise straight chain or branched chain hydrocarbons of 1 to 6 carbon atoms, or in the alternative, R¹INH may be absent;

R², R³ and R⁴ are the side chains of naturally occurring amino acids, or in the alternative may be substituted or unsubstituted aliphatic, aromatic or heteroaromatic groups, such as allyl, cyclohexyl, phenyl, pyridyl, imidazolyl or saturated chains of 2 to 8 carbon atoms, wherein the aliphatic substituents may be substituted with an aromatic or heteroaromatic ring; and

R⁵ is H or a straight or branched chain aliphatic group, which may be substituted with an aromatic or heteroaromatic group;

(f) compounds of the following formula, which compounds are also disclosed in U.S. Pat. No. 5,340,828, incorporated herein by reference,

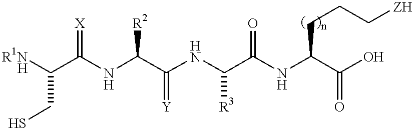

wherein:

X and Y are independently H₂ or O, provided that at least one of these is H₂;

R¹ is H, an alkyl group, an acyl group, an alkylsulfonyl group or aryl sulfonyl group, wherein alkyl and acyl groups comprise straight chain or branched chain hydrocarbons of 1 to 6 carbon atoms, or in the alternative, R¹NH may be absent;

R² and R³ are the side chains of naturally occurring amino acids, or in the alternative may be substituted or unsubstituted aliphatic, aromatic or heteroaromatic groups, such as allyl, cyclohexyl, phenyl, pyridyl, imidazolyl or saturated chains of 2 to 8 carbon atoms, wherein the.aliphatic substituents may be substituted with an aromatic or heteroaromatic ring;

Z is O or S; and

n is0, 1 or2;

(g) compounds of the following formula, which compounds are also disclosed in U.S. Pat. No. 5,340,828, incorporated herein by reference,

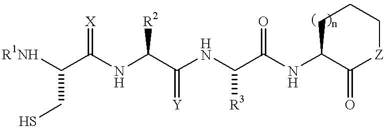

wherein:

X and Y are independently H₂ or O, provided that at least one of these is H₂;

R¹ is H, an alkyl group, an acyl group, an alkylsulfonyl group or aryl sulfonyl group, wherein alkyl and acyl groups comprise straight chain or branched chain hydrocarbons of 1 to 6 carbon atoms, or in the alternative, R¹NH may be absent;

R² and R³ are the side chains of naturally occurring amino acids, or in the alternative may be substituted or unsubstituted aliphatic, aromatic or heteroaromatic groups, such as allyl, cyclohexyl, phenyl, pyridyl, imidazolyl or saturated chains of 2 to 8 carbon atoms, wherein the aliphatic substituents may be substituted with an aromatic or heteroaromatic ring;

Z is O or S; and

n is 0, 1 or2;

(h) compounds of the following formula, which compounds are also disclosed in U.S. Pat. No. 5,352,705, incorporated herein by reference,

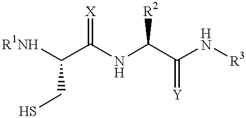

wherein:

X and Y are independently H₂O or O;

R¹ is an alkyl group, hydrogen, an acyl group, an alkylsulfonyl group or arylsulfonyl group, wherein alkyl and acyl groups comprise straight chain or branched chain hydrocarbons of I to 6 carbons atoms, which alternatively may be substituted with an aryl group;

R² is the side chains of naturally occurring amino acids, or in the alternative may be substituted or unsubstituted aliphatic, aromatic or heterocyclic groups, such as allyl, cyclohexyl, phenyl. pyridyl, imidazolyl or saturated chains of 2 to 8 carbon atoms which may be branched or unbranched, wherein the aliphatic substituents may be substituted with an aromatic or heteroaromatic ring;

R³ is an aromatic or heteroaromatic ring or in the alternative an alkyl group or an aryl or heteroaryl substituted alkane, wherein the aromatic ring is unsubstituted or in the alternative, substituted with one or more groups which may be alkyl, halo, alkoxy, trifluoromethyl, or sulfamoyl groups, and which may be polycyclic;

(i) compounds of the following formulae, which compounds are also disclosed in U.S. Pat. No. 5,326,773 and PCT Publication No. WO 94/10137, incorporated herein by reference,

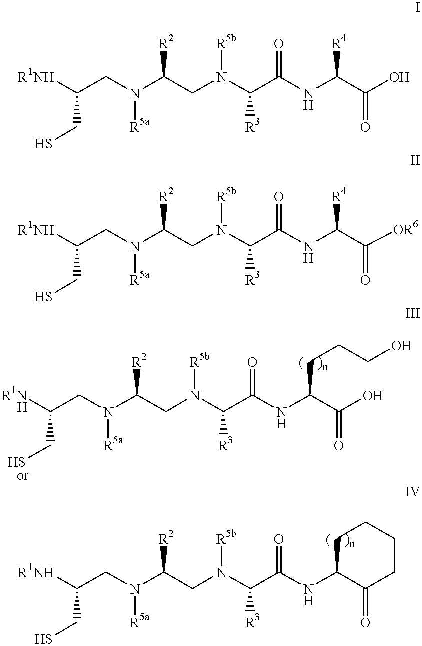

wherein in said formulae I, II, III and IV:

R¹ and R^{5a} are independently selected from hydrogen, a C₁-C₆ alkyl group, a C₁-C₆ acyl group, an aroyl group, a C₁-C₆ alkylsulfonyl group, C₁-C₆ aralkylsulfonyl group or arylsulfonyl group wherein the alkyl group and acyl group is optionally substituted with substituted or unsubstituted aryl or heterocycle; R², R³ and R⁴ are independently selected from:

a) a side chain of naturally occurring amino acids,

b) an oxidized form of a side chain of naturally occurring amino acids selected from methionine sulfoxide and methionine sulfone,

c) substituted or unsubstituted C₁-C₈ alkyl, C₃-C₈ cycloalkyl, C₂-C₈ alkenyl, aryl or heterocycle groups, wherein the aliphatic substituent is optionally substituted with an aryl, heterocycle or C₃-C₈ cycloalkyl;

R^{5b} is a C₁-C₆ alkyl group, a C₁-C₆ acyl group, an aroyl group, a C₁-C₆ alkylsulfonyl group, C₁-C₆ aralkylsulfonyl group or arylsulfonyl group wherein the alkyl group and acyl group is optionally substituted with substituted or unsubstituted aryl or heterocycle;

R⁶ is a substituted or unsubstituted aliphatic, aryl or heterocyclic group, wherein the aliphatic substituent is optionally substituted with an aryl or heterocyclic ring; and

n is 0, 1 or 2;

(j) compounds of the following formulae, which compounds are also disclosed in U.S. Pat. No. 5,504,212, incorporated herein by reference,

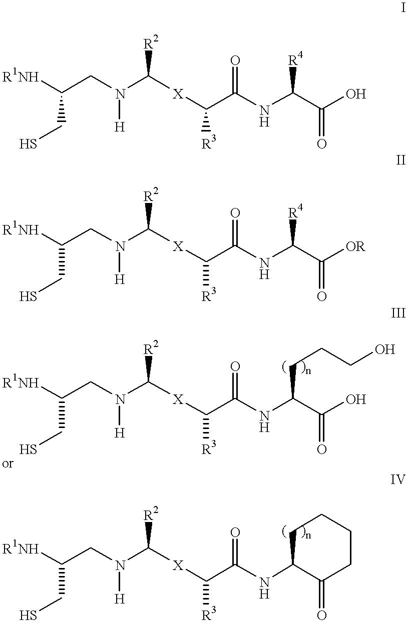

wherein in said formulae I, II, III and IV:

R¹ is selected from hydrogen, a C₁-C₆ alkyl group, a C₁-C₆ acyl group, an aroyl group, a C₁-C₆ alkylsulfonyl group, C₁-C₆ aralkylsulfonyl group or arylsulfonyl group wherein the alkyl group and acyl group is optionally substituted with substituted or unsubstituted aryl or heterocycle;

R², R³ and R⁴ are independently selected from:

a) a side chain of naturally occurring amino acids,

b) an oxidized form of a side chain of naturally occurring amino acids selected from methionine sulfoxide and methionine sulfone,

c) substituted or unsubstituted C₁-C₈ alkyl, C₃-C₈ cycloalkyl, C₂-C₈ alkenyl, aryl or heterocycle groups, wherein the aliphatic substituent is optionally substituted with an aryl, heterocycle or C₃-C₈ cycloalkyl;

X is CH₂CH₂ or trans CH═CH;

R⁶ is a substituted or unsubstituted aliphatic, aryl or heterocyclic group, wherein the aliphatic substituent is optionally substituted with an aryl or heterocyclic ring; and

n is 0, 1 or 2;

(k) compounds of the following formulae, which compounds are also disclosed in PCT Publication No. WO 94/10138, incorporated herein by reference,

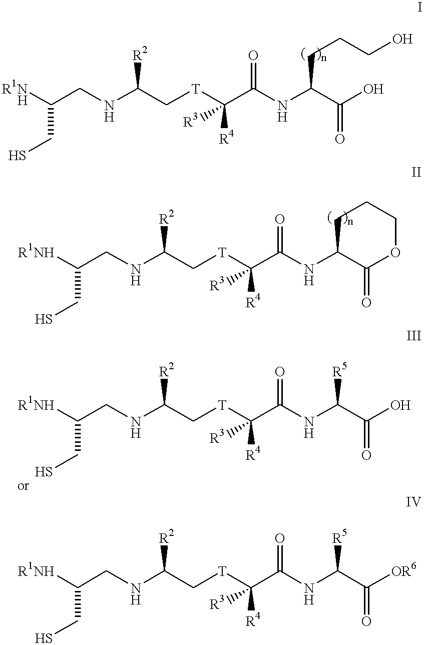

wherein in said formulae I, II, III and IV,

R¹ is hydrogen, an alkyl group, an aralkyl group, an acyl group, an aracyl group, an aroyl group, an alkylsulfonyl group, aralkylsulfonyl group or arylsulfonyl group, wherein alkyl and acyl groups comprise straight chain or branched chain hydrocarbons of 1 to 6 carbon atoms;

R², R³ and R⁵ are the side chains of naturally occurring amino acids, including their oxidized forms which may be methionine sulfoxide or methionine sulfone, or in the alternative may be substituted or unsubstituted aliphatic, aromatic or heteroaromatic groups, such as allyl, cyclohexyl, phenyl, pyridyl, imidazolyl or saturated chains of 2 to 8 carbon atoms which may be branched or unbranched, wherein the aliphatic substituents may be substituted with an aromatic or heteroaromatic ringf;

R⁴ is hydrogen or an alkyl group, wherein the alkyl group comprises straight chain or branched chain hydrocarbons of 1 to 6 carbon atoms;

R⁶ is a substituted or unsubstituted aliphatic, aromatic or heteroaromatic group such as saturated chains of 1 to 8 carbon atoms, which may be branched or unbranched, wherein the aliphatic substituent may be substituted with an aromatic or heteroaromatic ring,

T is O or S(O)ₘ;

is 0, 1 or 2; and

n is 0, 1 or 2;

(1) compounds of the following formulae, which compounds are also disclosed in PCT Publication No. WO 95/00497, incorporated herein by reference,

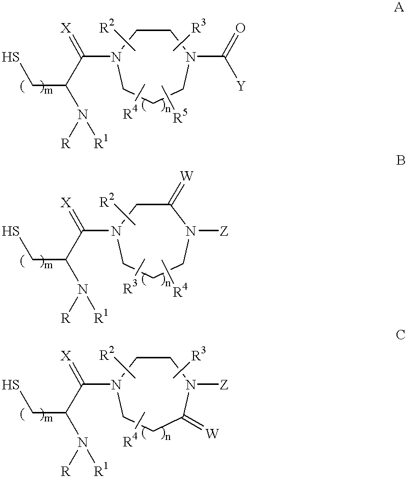

wherein in said formulae A, B and C:

X is O or H₂;

m is 1 or 2;

n is 0 or 1;

t is 1 to 4;

R and R¹ are independently selected from H, C₁₋₄ alkyl, or aralkyl;

R², R³, R⁴, and Rs are independently selected from H, C₁₋₈ alkyl, alkenyl,

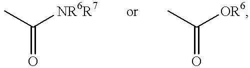

alkynyl, aryl, heterocycle, unsubstituted or substituted with one or more of:

1) aryl or heterocycle, unsubstituted or substituted with:

a) C₁₋₄ alkyl,

b) (CH₂)ₜOR⁶,

c) (CH₂)ₜNR⁶R⁷,

d) halogen,

2) C₃₋₆ cycloalkyl,

3) OR⁶,

4) SR⁶, S(O)R⁶, SO₂R⁶,

5) —NR⁶R⁷,

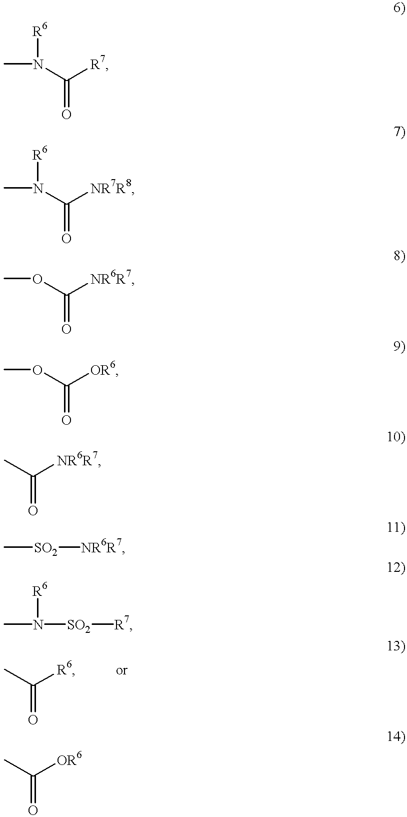

and any two of R², R³, R⁴, and R⁵ are optionally attached to the same carbon atom;

Y is aryl, heterocycle, unsubstituted or substituted with one or more of:

1) C₁₋₄ alkyl, unsubstituted or substituted with:

a) C₁₋₄ alkoxy,

b) NR⁶R⁷,

c) C₃₋₆ cycloalkyl,

d) aryl or heterocycle,

e) HO,

2) aryl or heterocycle,

3) halogen,

4) OR⁶,

5) NR⁶R ,

6) CN,

7) NO₂, or

8) CF₃;

W is H₂ or O;

Z is aryl, heteroaryl, arylmethyl, heteroarylmethyl, arylsulfonyl, heteroarylsulfonyl, unsubstituted or substituted with one or more of the following:

1) C₁₋₄ alkyl, unsubstituted or substituted with:

a) C₁₋₄ alkoxy,

b) NR⁶R,

c) C₃₋₆ cycloalkyl,

d) aryl or heterocycle, or

e) HO,

2) aryl or heterocycle,

3) halogen,

4) OR⁶,

5) NR⁶R⁷,

6) CN,

7) NO₂, or

8) CF₃;

R⁶, R⁷ and R⁸ are independently selected from H, C₁₋₄ alkyl, C₃₋₆ cycloalkyl, heterocycle, aryl, aroyl, heteroaroyl, arylsulfonyl, heteroarylsulfonyl, unsubstituted or substituted with:

a) C₁₋₄ alkoxy,

b) aryl or heterocycle,

c) halogen,

d) HO,

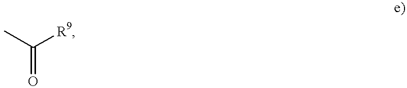

f) —SO₂R⁹, or

g) NRR¹, wherein

R⁶ and R may be joined in a ring, and

R and R¹ may be joined in a ring; and

R⁹ is C₁₋₄ alkyl or aralkyl.

(m) compounds of the following formulae, which compounds are ,so disclosed in PCT Publication No. WO 96/09821, incorporated herein by reference,

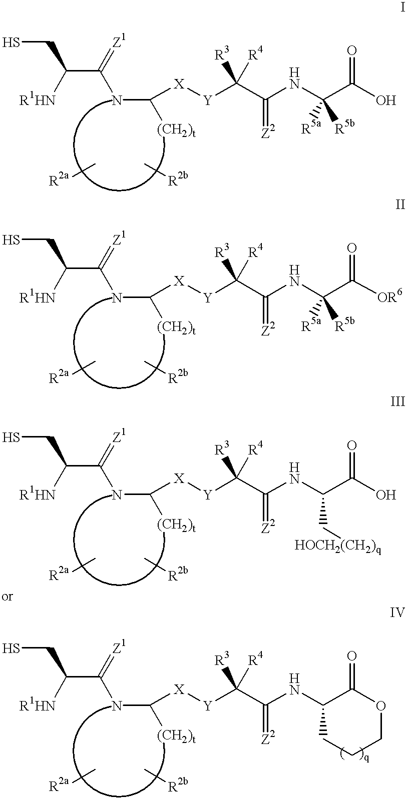

wherein in said formulae I, II, III and IV:

R¹ is selected from:

a) hydrogen,

b) R⁸S(O)₂—, R⁸C(O)—, (R⁸)₂NC(O)— or R⁹OC(O)—, and

c) C₁-C₆ alkyl unsubstituted or substituted by aryl, heterocyclic, cycloalkyl, alkenyl, alkynyl, R⁸O—, R⁸S(O)ₘ—, R⁸C(O)NR⁸-, CN, (R⁸)₂N—C(NR⁸)—, R⁸C(O)—, R⁸OC(O)—, N₃, —N(R⁸)₂, or R⁹OC(O)NR⁸-;

R^{2a} and R^{2b} are independently selected from:

a) hydrogen,

b) C₁-C₆ alkyl unsubstituted or substituted by alkenyl, R⁸O—, R⁸S(O)ₘ—, R⁸C(O)NR—, CN, (R⁸)₂N—C(NR⁸)—, R⁸C(O)—, R⁸OC(O)—, N₃, —N(R⁸)₂, or R⁹OC(O)NR⁸-,

c) aryl, heterocycle, cycloalkyl, alkenyl, R⁸O—, R⁸S(O)ₘ—, R⁸C(O)NR⁸-, CN, NO₂, (R⁸)₂NC(NR⁸)—, R⁸C(O)—, R⁸OC(O)—, N₃, —N(R⁸)₂, or R⁹OC(O)NR⁸-, and

d) C₁-C₆ alkyl substituted with an unsubstituted or substituted group selected from aryl, heterocyclic and C₃-C₁₀ cycloalkyl;

R³ and R⁴ are independently selected from:

a) a side chain of a naturally occurring amino acid,

b) an oxidized form of a side chain of a naturally occurring amino acid which is:

i) methionine sulfoxide, or

ii) methionine sulfone, and

c) substituted or unsubstituted C₁-C₂₀ alkyl, C₂-C₂₀ alkenyl, C₃-C₁₀ cycloalkyl, aryl or heterocyclic group, wherein the substituent is selected from F, Cl, Br, N(R⁸)₂, NO₂, R⁸O—, R⁸S(O)ₘ—, R⁸C(O)NR⁸-, CN, (R⁸)₂N—C(NR⁸)—, R⁸C(O)—, R¹OC(O)—, N₃, —N(R⁸)₂, R⁹OC(O)NR⁸- and C₁-C₂₀ alkyl, and

d) C₁-C₆ alkyl substituted with an unsubstituted or substituted group selected from aryl, heterocyclic and C₃-C₁₀ cycloalkyl; or

R³ and R⁴ are combined to form —(CH₂)ₛ—;

R^{5a} and R^{5b} are independently selected from:

a) a side chain of a naturally occurring amino acid,

b) an oxidized form of a side chain of a naturally occurring amino acid which is:

i) methionine sulfoxide, or

ii) methionine sulfone,

c) substituted or unsubstituted C₁-C₂₀ alkyl, C₂-C₂₀ alkenyl, C₃-C₁₀ cycloalkyl, aryl or heterocycle group, wherein the substituent is selected from F, Cl, Br, N(R⁸)₂, NO₂, R⁸O—, R¹S(O)ₘ—, R⁸C(O)NR⁸-, CN, (R⁸)₂N—C(NR⁸)—, R⁸C(O)—, R⁸OC(O)—, N₃, —N(R⁸)₂, R⁹OC(O)NR⁸- and C₁-C₂₀ alkyl, and

d) C₁-C₆ alkyl substituted with an unsubstituted or substituted group selected from aryl, heterocyclic and C₃-C₁₀ cycloalkyl; or

R^{5a} and R^{5b} are combined to form —(CH₂)ₛ— wherein one of the carbon atoms is optionally replaced by a moiety selected from O, S(O)ₘ, —NC(O)—, and —N(COR⁸)—;

R⁶ is

a) substituted or unsubstituted C₁-C₈ alkyl, wherein the substituent on the alkyl is selected from:

1) aryl,

2) heterocycle,

3) —N(R⁸)₂,

4) —OR⁸, or

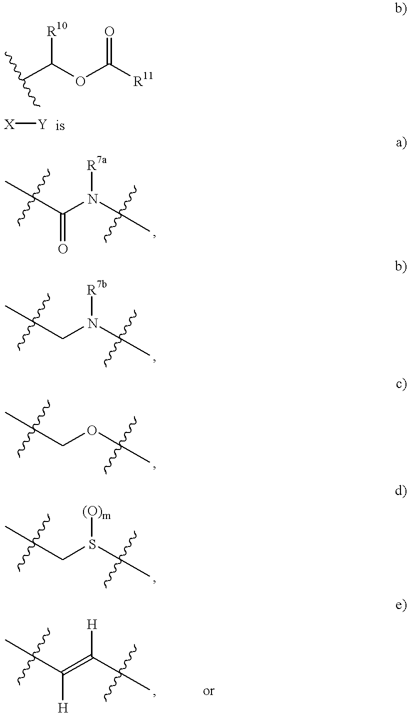

f) —CH₂—CH₂—

R⁷, is selected from

a) hydrogen,

b) unsubstituted or substituted aryl,

c) unsubstituted or substituted heterocycle,

d) unsubstituted or substituted cycloalkyl, and

e) C₁-C₆ alkyl substituted with hydrogen or an unsubstituted or substituted group selected from aryl, heterocycle and cycloalkyl;

R^{7b} is selected from:

a) hydrogen,

b) unsubstituted or substituted aryl,

c) unsubstituted or substituted heterocycle,

d) unsubstituted or substituted cycloalkyl,

e) C₁-C₆ alkyl substituted with hydrogen or an unsubstituted or substituted group selected from aryl, heterocycle and cycloalkyl,

f) a carbonyl group which is bonded to an unsubstituted or substituted group selected from aryl, heterocycle, cycloalkyl and C₁-C₆ alkyl substituted with hydrogen or an unsubstituted or substituted group selected from aryl, heterocycle and cycloalkyl, and

g) a sulfonyl group which is bonded to an unsubstituted or substituted group selected from aryl, heterocycle, cycloalkyl and C₁-C₆ alkyl substituted with hydrogen or an unsubstituted or substituted group selected from aryl, heterocycle and cycloalkyl;

R⁸ is independently selected from hydrogen, C₁-C₆ alkyl and aryl;

R⁹ is independently selected from C₁-C₆ alkyl and aryl;

R¹⁰ is independently selected from hydrogen and C₁-C₆ alkyl;

R¹¹ is independently selected from C₁-C₆ alkyl;

Z¹ and Z² are independently H₂ or O, provided that Z¹ is not 0 when X—Y is —C(O)N(R^{7a});

m is 0, 1 or 2;

q is 0, 1 or2;

s is 4 or 5; and

t is 3, 4 or 5;

(n) compounds of the following formulae, which compounds are also disclosed in PCT Publication No. WO 96109820, incorporated herein by reference,

[chemical structure]

wherein:

R¹ is selected from:

a) hydrogen,

b) R⁵S(O)₂—, R⁵C(O)—, (R⁵)₂NC(O)— or R⁶OC(O)—, and

c) C₁-C₆ alkyl unsubstituted or substituted by aryl, heterocyclic, cycloalkyl, alkenyl, alkynyl, R⁵O—, R⁵S(O)ₘ—, R⁵C(O)NR⁵—, CN, (R⁵)₂N—R⁵)—, R⁵C(O)—, R⁵OC(O)—, N₃, —N(R⁵)₂, or R⁶OC(O)N⁵—;

R^{2a} and R^{2b} are independently selected from:

a) hydrogen,

b) C₁-C₆ alkyl unsubstituted or substituted by aryl, heterocycle, cycloalkyl, alkenyl, R⁵O—, R⁵S(O)ₘ—, R⁵C(O)NR⁵-CN, (R⁵)₂N—C(NR⁵)—, R⁵C(O)—, R⁵OC(O)—, N₃, —N(R⁵)₂, or R⁶OC(O)Nk⁵-, and

c) aryl, heterocycle, cycloalkyl, alkenyl, R⁵O—, R⁵S(O)MR⁵C(O)NR⁵—, CN, NO₂, (R⁵)₂N—C(NR⁵)—, R⁵C(O)—. R¹OC(O)—, N₃, —N(R⁵)₂, or R⁶OC(O)NR⁵—,

R³ is selected from:

a) unsubstituted or substituted aryl,

b) unsubstituted or substituted heterocycle,

c) unsubstituted or substituted cycloalkyl, and

d) C₁-C₆ alkyl substituted with hydrogen or an unsubstituted or substituted group selected from aryl, heterocycle and cycloalkyl;

X—Y is

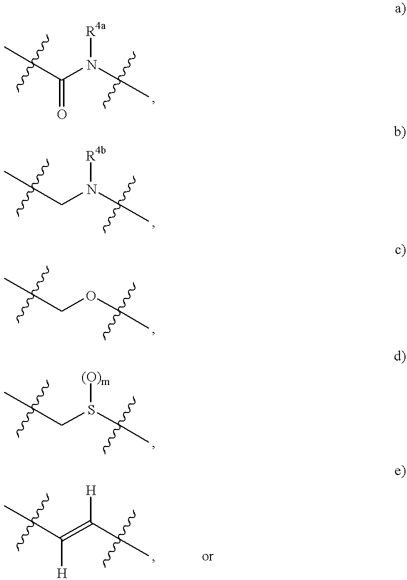

f) —CH₂—CH₂—

R^{4a} is selected from

a) hydrogen,

b) unsubstituted or substituted aryl,

c) unsubstituted or substituted heterocycle,

d) unsubstituted or substituted cycloalkyl, and

e) C₁-C₆ alkyl substituted with hydrogen or an unsubstituted or substituted group selected from aryl, heterocycle and cycloalkyl;

R^{4b} is selected from

a) hydrogen,

b) unsubstituted or substituted aryl,

c) unsubstituted or substituted heterocycle,

d) unsubstituted or substituted cycloalkyl,

e) C₁-C₆ alkyl substituted with hydrogen or an unsubstituted or substituted group selected from aryl, heterocycle and cycloalkyl,

f) a carbonyl group which is bonded to an unsubstituted or substituted group selected from aryl, heterocycle, cycloalkyl and C₁-C₆ alkyl substituted with hydrogen or an unsubstituted or substituted group selected from aryl, heterocycle and cycloalkyl, and

g) a sulfonyl group which is bonded to an unsubstituted or substituted group selected from aryl, heterocycle, cycloalkyl and C₁-C₆ alkyl substituted with hydrogen or an unsubstituted or substituted group selected from aryl, heterocycle and cycloalkyl;

R⁵ is independently selected from hydrogen, C₁-C₆ alkyl and aryl;

R⁶ is independently selected from C₁-C₆ alkyl and aryl;

Z is independently H₂O or O;

m is 0, 1 or 2, provided that m is 0 when R⁵=hydrogen;

n is0, 1, 2, 3 or 4; and

t is 3, 4 or 5.

(o) compounds of the following formulae:

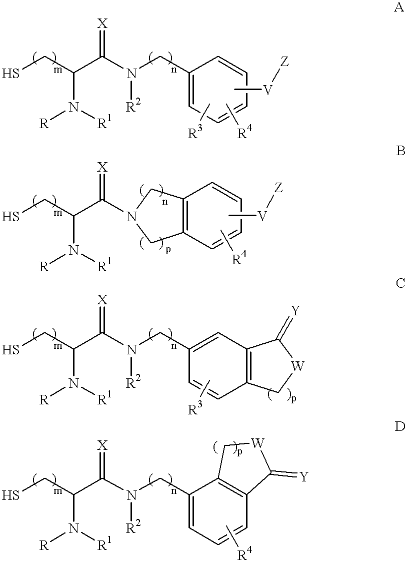

wherein in said formula A, B, C and D:

X and Y are independently O or H₂O;

m is 1 or 2;

n is O or 1;

p is 1, 2 or 3;

q is 0, 1 or 2;

t is 1 to 4;

R, R¹ and R² are independently selected from H, C₁₋₆ alkyl, or C₁₋₆ aralkyl;

R³ and R⁴ are independently selected from:

a) hydrogen,

b) C₁-C₆ alkyl unsubstituted or substituted by C₂-C₆ alkenyl, R⁶O—, R⁵S(O)_{q}—, R⁷C(O)NR⁶-, CN, N₃, R⁶OC(O)NR⁶-, R⁶R⁷N—C(NR⁶R⁸)—, R⁶C(O)—, R⁷R⁸NC(O)O—, R⁷R⁸NC(O)—, R⁶R⁷N-S(0)₂-, —Nk⁶S(0)₂R⁵, R⁶OC(0)0-, —Nk⁶R⁷, or R⁷R⁸NC(O)NR⁶,

c) unsubstituted or substituted cycloalkyl, alkenyl, R⁶0-, R⁵S(O)_{q}—, R⁶C(O)NR⁶-, CN, NO₂, R⁶R⁷N—C(NR⁸)—, R⁶C(O)—, N₃, —NR⁶R⁷, halogen or R⁷OC(O)NR⁶-, and

d) C₁-C₆ alkyl substituted with an unsubstituted or substituted group selected from aryl, heterocyclic and C₃-C₁₀ cycloalkyl;

W is —CHR⁹-or —NR⁹-;

Z is unsubstituted or substituted C₁₀₈ alkyl, unsubstituted or substituted C₂₋₈ alkenyl, unsubstituted or substituted aryl, unsubstituted or substituted heterocycle; wherein the substituted group is substituted with one or more of:

1) C₁₋₄ alkyl, unsubstituted or substituted with:

a) C₁₋₄ alkoxy,

b) N—R⁶R⁷,

c) C₃₋₆ cycloalkyl,

d) aryl or heterocycle,

e) HO,

2) aryl or heterocycle,

3) halogen,

4) OR⁶,

5) NR⁶R⁷,

6) CN,

7) NO₂, or

9) CF₃;

R⁵ is C₁₋₄ alkyl or aralkyl;

R⁶, R⁷ and R⁸ are independently selected from H, C₁₋₄ alkyl, C₃₋₆ cycloalkyl, heterocycle, aryl, aroyl, heteroaroyl, arylsulfonyl, heteroarylsulfonyl, unsubstituted or substituted with:

a) C₁₋₄ alkoxy,

b) aryl or heterocycle,

c) halogen,

d) HO,

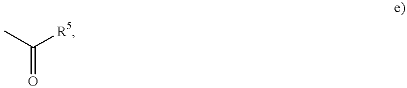

f) —SO₂R⁵, or

g) —NR⁶R⁷, or

R⁶ and R⁷ may be joined in a ring, and

R¹ and RS may be joined in a ring;

R⁹ is selected from H, Cl, alkyl, C₃₋₆ cycloalkyl, heterocycle and aryl, unsubstituted, monosubstituted or disubstituted with substituents independently selected from:

a) C₁₋₄ alkyl,

b) C₁₋₄alkoxy,

c) aryl or heterocycle,

d) halogen,

e) HO,

f)

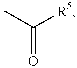

g) —SO₂R³, and

h) —NR⁶R⁷;

V is selected from —C(R¹¹)═C(R¹¹)—, C—C—, —C(O)—, —C(R¹¹)₂—, —C(OR¹¹)R¹¹-, —CN(R¹¹)₂R¹¹-, —OC(R¹¹)2—, —NR¹¹C(R¹¹)₂—, —C(R¹¹)₂0-, —C(R¹¹)₂NR¹¹-, —C(O)NR¹¹-, —NR¹¹C(O)—, 0, —NC(O)R¹¹-, —NC(O)0R¹¹-, —S(O)₂N(R¹¹)—, —N(R¹¹)S(O)₂—, or S(O)ₘ;

R¹⁰ and R¹¹ are independently selected from hydrogen, C₁-C₆ alkyl, C₂-C₄ alkenyl, benzyl and aryl; or the pharmaceutically acceptable salt thereof.

Compounds suitable for use in the methods of the invention also include those farnesyl-protein transferase inhibitors that do not incorporates a cysteinyl or sulfhydryl containing moiety at the N terminus of the molecule. Such compounds may exhibit preferred pharmacological activity, e.g. by avoiding thiol-related reactions in vivo. More specifically, the following compounds may be suitable.

(p) compounds of the following formulae, which compounds are also disclosed in PCT Publication No. WO 95/09001, incorporated herein by reference,

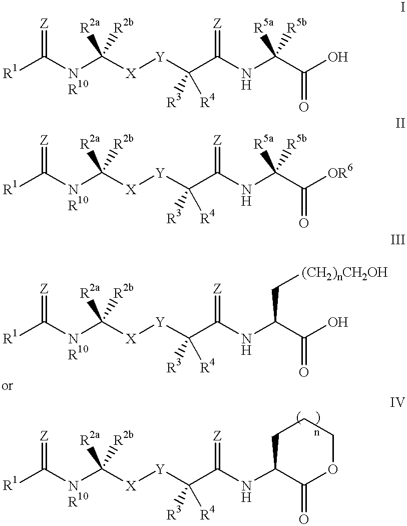

wherein in said formulae I, II, III and IV:

R¹ is selected from:

a) heterocycle, and

b) C₁-C₁₀ alkyl, which is substituted with heterocycle and which is optionally substituted with one or more of C₁-C₄ alkyl, hydroxy or amino groups;

R^{2a} and R^{2b} are independently selected from:

a) a side chain of a naturally occurring amino acid,

b) an oxidized form of a side chain of a naturally occurring amino acid which is:

i) methionine sulfoxide, or

ii) methionine sulfone,

c) substituted or unsubstituted C₁-C alkyl, C₂-C₂₀ alkenyl, C₃-C₁₀ cycloalkyl, aryl or heterocyclic group, wherein the substituent is selected from F, Cl, Br, NO₂, R⁸O—, R⁹S(O)ₘ—, R⁸C(O)NR⁸-, CN, (R⁸)₂NC(NR⁸)—, R⁸C(O)—, R⁸OC(O)—, N₃, —N(R⁸)₂, R⁹OC(O)NR⁸- and C₁-C₂₀ alkyl, and

d) C₁-C₆ alkyl substituted with an unsubstituted or substituted group selected from aryl, heterocycle and C₃-C₁₀ cycloalkyl; or

R^{2a} and R^{2b} are combined to form —(CH₂)ₛ—;

R³ and R⁴ are independently selected from:

a) a side chain of a naturally occurring amino acid,

b) an oxidized form of a side chain of a naturally occurring amino acid which is:

i) methionine sulfoxide, or

ii) methionine sulfone, and

c) substituted or unsubstituted C₁-C₂₀ alkyl, C₂-C₂₀ alkenyl, C₃-C₁₀ cycloalkyl, aryl or heterocyclic group, wherein the substituent is selected from F, Cl, Br, N(R⁸)₂, NO₂, R⁸O—, R⁹S(O)ₘ—, R⁸C(O)NR⁸-, CN, (R⁸)₂N—C(NR⁸)—, R⁸C(O)—, R⁸OC(O)—, N₃, —N(R⁸)₂, R⁹OC(O)NR⁸- and C₁-C₂₀ alkyl, and

d) C₁-C₆ alkyl substituted with an unsubstituted or substituted group selected from aryl, heterocyclic and C₃₋₁₀ cycloalkyl; or

R³ and R⁴ are combined to form —(CH₂)ₛ—;

R^{5a} and R^{5b} are independently selected from:

a) a side chain of a naturally occurring amino acid,

b) an oxidized form of a side chain of a naturally occurring amino acid which is:

i) methionine sulfoxide, or

ii) methionine sulfone,

c) substituted or unsubstituted C₁-C₂₀ alkyl, C₂-C₂₀ alkenyl, C₃-C₁₀ cycloalkyl, aryl or heterocycle group, wherein the substituent is selected from F, Cl, Br, N(R⁸)₂, NO₂, R⁸O—, R⁹S(O)ₘ—, R⁸C(O)NR⁸-, CN, (R⁸)₂N—C(NR⁸)—, R⁸C(O)—, R⁸OC(O)—, N₃, —N(R⁸)₂, R⁹OC(O)NR⁸- and C₁-C₂₀ alkyl, and

d) C₁-C₆ alkyl substituted with an unsubstituted or substituted group selected from aryl, heterocyclic and C₃-C₁₀ cycloalkyl; or

R^{5a} and R^{5b} are combined to form —(CH₂)ₛ— wherein one of the carbon atoms is optionally replaced by a moiety selected from O, S(O)ₘ, —NC(O)—, and —N(COR⁸)—; R⁶ is

a) substituted or unsubstituted C₁-C₈ alkyl, wherein the substituent on the alkyl is selected from:

1) aryl,

2) heterocycle,

3) —N(R⁹)₂,

4) —OR⁸ or

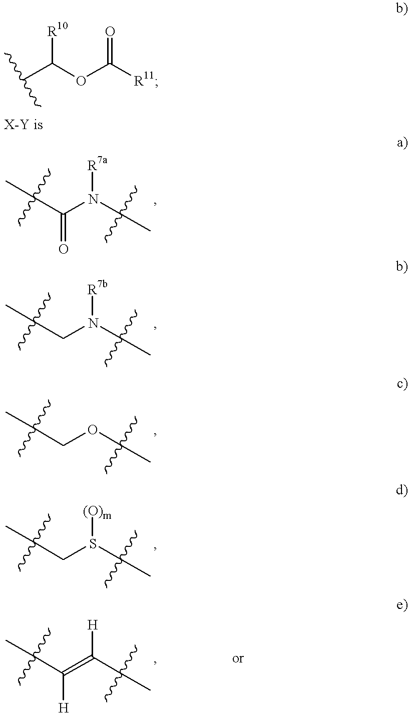

f) —CH₂—CH₂—;

R^{7a} is selected from

a) hydrogen,

b) unsubstituted or substituted aryl,

c) unsubstituted or substituted heterocyclic,

d) unsubstituted or substituted cycloalkyl, and

e) C₁-C₆ alkyl substituted with hydrogen or an unsubstituted or substituted group selected from aryl, heterocyclic and cycloalkyl;

R^{7b} is selected from

a) hydrogen,

b) unsubstituted or substituted aryl,

c) unsubstituted or substituted heterocyclic,

d) unsubstituted or substituted cycloalkyl,

e) C₁-C₆ alkyl substituted with hydrogen or an unsubstituted or substituted group selected from aryl, heterocyclic and cycloalkyl,

f) a carbonyl group which is bonded to an unsubstituted or substituted group selected from aryl, heterocyclic, cycloalkyl and C₁-C₆ alkyl substituted with hydrogen or an unsubstituted or substituted group selected from aryl, heterocyclic and cycloalkyl, and

g) a sulfonyl group which is bonded to an unsubstituted or substituted group selected from aryl, heterocyclic, cycloalkyl and C₁-C₆ alkyl substituted with hydrogen or an unsubstituted or substituted group selected from aryl, heterocyclic and cycloalkyl;

R⁸ is independently selected from hydrogen, C₁-C₆ alkyl and aryl;

R⁹ is independently selected from C₁-C₆ alkyl and aryl;

R¹⁰ is independently selected from hydrogen and C₁-C₆ alkyl;

R¹¹ is independently selected from C₁-C₆ alkyl;

Z is independently H₂ or O;

mis 0, 1 or2;

n is 0, 1 or 2; and

s is 4 or 5;

(q) compounds of the following formulae, which compounds are also disclosed in PCT Publication No. WO 95/09000 and U.S. Pat. No. 5,468,773, incorporated herein by reference,

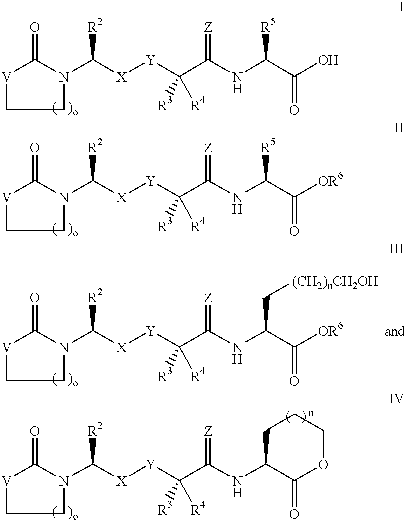

wherein in said formula I, II, III and IV:

V is CH₂, O, S, HN, or R⁷N;

R², R³, R⁴ and R⁵ are independently the side chains of naturally occurring amino acids, including their oxidized forms which may be methionine sulfoxide or methionine sulfone, or in the alternative may be substituted or unsubstituted aliphatic, aromatic or heteroaromatic groups, such as allyl, cyclohexyl, phenyl, pyridyl, imidazolyl or saturated chains of 2 to 8 carbon atoms which may be branched or unbranched, wherein the aliphatic substituents may be substituted with an aromatic or heteroaromatic ring;

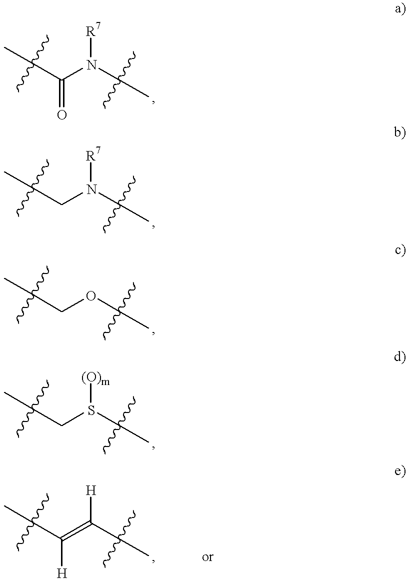

f) —CH₂—CH₂—

R⁶ is a substituted or unsubstituted aliphatic, aromatic or heteroaromatic group such as saturated chains of 1 to 8 carbon atoms, which may be branched or unbranched, wherein the aliphatic substituent may be substituted with an aromatic or heteroaromatic ring;

R⁷ is an alkyl group, wherein the alkyl group comprises straight chain or branched chain hydrocarbons of 1 to 6 carbon atoms, which may be substituted with an aromatic or heteroaromatic group;

Z is H₂or O;

m is 0, 1 or2;

n is 0, 1 or 2; and

o is 0, 1, 2 or 3;

(r) compounds of tbe following formulae, which compounds are also disclosed in PCT Publication No. WO 96/09836, incorporated herein by reference

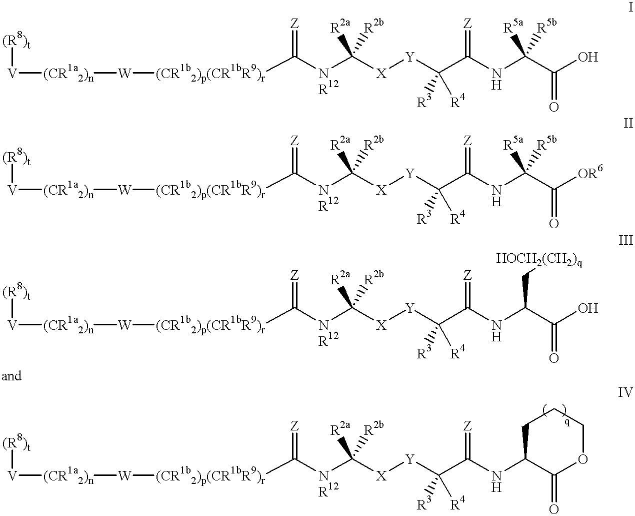

wherein in said forrnulae I, II, III and IV:

R is selected from:

a) hydrogen,

b) aryl, heterocyclic, cycloalkyl, alkenyl, alkynyl, (R¹⁰)₂N—C(NR¹)—, R¹⁰C(O)—, or R¹⁰OC(O)—, and

c) C₁-C₆ alkyl unsubstituted or substituted by aryl, heterocyclic, cycloalkyl, alkenyl, alkynyl, R¹⁰O—, R¹¹S(O)ₘ—, R¹⁰C(O)NR¹⁰-, CN, (R¹⁰)₂N—C(NR¹⁰)—, R¹⁰C(O)—, R¹⁰OC(O)—, N₃, —N(R¹⁰)₂, or R¹¹OC(O)NR¹⁰-;

R^{1b} is independently selected from:

a) hydrogen,

b) unsubstituted or substituted aryl, cycloalkyl, alkenyl, alkynyl, (R¹⁰)₂N—C(NR¹⁰)—, R¹⁰C(O)—, or RROC(O)—, and

c) C₁-C₆ alkyl unsubstituted or substituted by unsubstituted or substituted aryl, cycloalkyl, alkenyl, alkynyl, R¹⁰O—, R¹¹S(O)ₘ—, R¹⁰C(O)NR¹⁰-, CN, (R¹⁰)₂N—C(NR¹⁰)—, R¹⁰C(O)—, R¹⁰OC(O)—, N₃, —N(R¹⁰)₂, or R¹¹OC(O)NR¹⁰-;

provided that R^{1b} is not R¹⁰C(O)NR¹⁰-when R^{1a} is alkenyl,

V is hydrogen and X—Y is C(O)NR^{7a};

R^{2a} and R^{2b} are independently selected from:

a) a side chain of a naturally occurring amino acid,

b) an oxidized form of a side chain of a naturally occurring amino acid which is:

i) methionine sulfoxide, or

ii) methionine sulfone,

c) substituted or unsubstituted C₁-C₂₀ alkyl, C₂-C₂₀ alkenyl, C₃-C₁₀ cycloalkyl, aryl or heterocyclic group, wherein the substituent is selected from F. Cl, Br,

NO₂, R¹⁰O—, R¹(O)ₘ—, R¹⁰C(O)NR¹⁰O—, CN, (R¹⁰)₂N—C(NR¹⁰)—, R¹⁰C(O)—, R¹⁰C(O)—, N₃, —N(R¹⁰)₂, R¹¹OC(O)NR¹⁰- and C₁-C₂₀ alkyl, and

d) C₁-C₆ alkyl substituted with an unsubstituted or substituted group selected from aryl, heterocycle and C₃-C₁₀ cycloalkyl; or

R^{2a} and R^{2b} are combined to form —(CH₂)ₛ—;

R³ and R⁴ are independently selected from:

a) a side chain of a naturally occurring amino acid,

b) an oxidized form of a side chain of a naturally occurring amino acid which is:

i) methionine sulfoxide, or

ii) methionine sulfone,

c) substituted or unsubstituted C₁-C₂₀ alkyl, C₂-C₂₀ alkenyl, C₃-C₁₀ cycloalkyl, aryl or heterocyclic group, wherein the substituent is selected from F, Cl, Br, N(R¹⁰)₂, NO₂, R¹⁰O—, R¹¹S(O)ₘ—, R¹⁰C(O)NR¹⁰-, CN, (R¹⁰)₂N—C(NR¹⁰)—, R¹⁰C(O)—, R¹⁰OC(O)—, N₃, —N(R¹⁰)₂, R¹¹OC(O)NR¹⁰- and C₁-C₂₀ alkyl, and

d) C₁-C₆ alkyl substituted with an unsubstituted or substituted group selected from aryl, heterocycle and C₃-C₁₀ cycloalkyl; or

R³ and R⁴ are combined to form —(CH₂)ₛ—;

R^{5a} and R^{5b} independently selected from:

a) a side chain of a naturally occurring amino acid,

b) an oxidized formn of a side chain of a naturally occurring amino acid which is:

i) methionine sulfoxide, or

ii) methionine sulfone,

c) substituted or unsubstituted C₁-C₂₀ alkyl, C₂-C₂₀ alkenyl, C₃-C₁₀ cycloalkyl, aryl or heterocyclic group, wherein the substituent is selected from F, Cl, Br, NO₂, R¹⁰O—, R¹¹S(O)ₘ—, R¹⁰OC(O)NR¹⁰-, CN, (R¹⁰)₂N—CNR¹⁰)—, R¹⁰C(O)—, R¹⁰OC(9)—, N₃, —N(R¹⁰)₂, R¹¹C(O)NR¹⁰- and C₁-C₂₀ alkyl, and

d) C₁-C₆ alkyl substituted with an unsubstituted or substituted group selected from aryl, heterocycle and C₃-C₁₀ cycloalkyl; or

R^{5a} and R^{5b} are combined to form —(CH₂)s— wherein one of the carbon atoms is optionally replaced by a moiety selected from O, S(O)ₘ, —NC(O)—, and —N(COR¹⁰)—;

R⁶ is

a) substituted or unsubstituted C₁-C₈ alkyl, wherein the substituent on the alkyl is selected from:

1) aryl,

2) heterocycle,

3) —N(R¹¹)₂,

4) —OR¹⁰, or

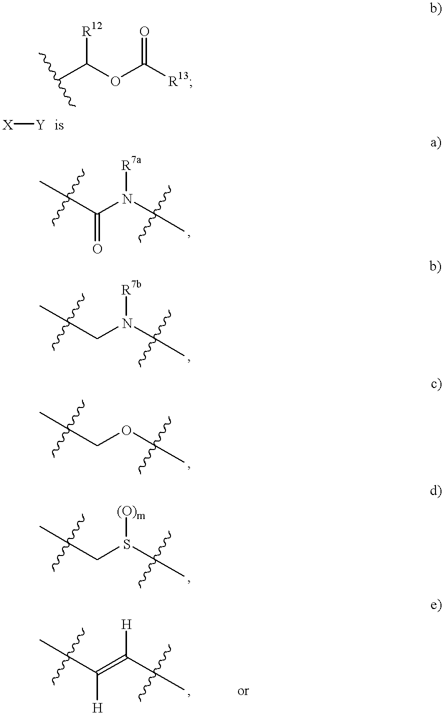

f) —CH₂—CH₂—

R^{7a} is selected from

a) hydrogen,

b) unsubstituted or substituted aryl,

c) unsubstituted or substituted heterocyclic,

d) unsubstituted or substituted cycloalkyl, and

e) C₁-C₆ alkyl substituted with hydrogen or an unsubstituted or substituted group selected from aryl, heterocyclic and cycloalkyl;

R^{7b} is selected from:

a) hydrogen,

b) unsubstituted or substituted aryl,

c) unsubstituted or substituted heterocyclic,

d) unsubstituted or substituted cycloalkyl,

e) C₁-C₆ alkyl substituted with hydrogen or an unsubstituted or substituted group selected from aryl, heterocyclic and cycloalkyl,

f) a carbonyl group which is bonded to an unsubstituted or substituted group selected from aryl, heterocyclic, cycloalkyl and C₁-C₆ alkyl substituted with hydrogen or an unsubstituted or substituted group selected from aryl, heterocyclic and cycloalkyl, and

g) a sulfonyl group which is bonded to an unsubstituted or substituted group selected from aryl, heterocyclic, cycloalkyl and C₁-C₆ alkyl substituted with hydrogen or an unsubstituted or substituted group selected from aryl, heterocyclic and cycloalkyl;

R⁸ is independently selected from:

a) hydrogen,

b) aryl, heterocyclic, cycloalkyl, alkenyl, alkynyl, perfluoroalkyl, F, Cl, Br, R¹⁰O—, R¹¹S(O)ₘ—, R¹⁰C(O)NR¹⁰-, CN, NO₂, R¹⁰2N—C(NR¹⁰)—, R¹⁰C(O)—, R¹⁰OC(O)—, N₃, —N(R¹⁰)₂, or R¹¹OC(O)NR¹⁰-, and

c) C₁-C₆ alkyl unsubstituted or substituted by aryl, heterocyclic, cycloalkyl, alkenyl, alkynyl, perfluoroalkyl, F, Cl, Br, R¹⁰O—, R¹¹S(O)ₘ—, R¹OC(O)NH—, CN, H₂N—C(NH)—, R¹⁰C(O)—, R¹⁰OC(O)—, N₃, —N(R⁸)₂, or R¹¹OC(O)NH—;

R⁹ is selected from:

hydrogen, C₁-C₆ alkyl, R¹⁰O—, R¹¹S(O)ₘ—, R¹OC(O)NR¹⁰-, CN, NO₂, N₃, —N(R¹⁰)₂, and R¹¹OC(O)NR¹⁰-;

provided that R⁹ is not R¹⁰C(O)NR¹⁰-when R^{1a} is alkenyl, V is hydrogen and X—Y is —C(O)NR⁷-;

R¹⁰ is independently selected from hydrogen, C₁-C₆ alkyl, benzyl and aryl;

R¹¹ is independently selected from C₁-C₆ alkyl and aryl;

R¹² is independently selected from hydrogen and C₁-C₆ alkyl;

R¹³ is C₁-C₆ alkyl;

V is selected from:

a) aryl;

b) heterocycle; or

c) hydrogen;

W is —S(O)ₘ—, —O—, —NHC(O)—, —C(O)NH—, —NHSO₂—, —SO₂NH—, N(R^{7a})— or N[C(O)R^{7a}];

Z is independently H₂ or O;

m is 0, 1 or 2;

n is 0, 1, 2, 3 or 4, provided that n is not 0 when V is hydrogen and W is —S(O)ₘ;

p is 0, 1, 2, 3 or 4, provided that p=0 when R⁹ is not hydrogen or C₁-C₆ lower alkyl;

q is 0, 1 or 2;

r is 0 or 1;

s is 4 or 5; and

t is 0, 1 or 2, provided that t=0 when V is hydrogen;

(s) compounds of the following formulae which compounds are also disclosed in PCT Publication WO 96/10011, incorporated herein by reference,

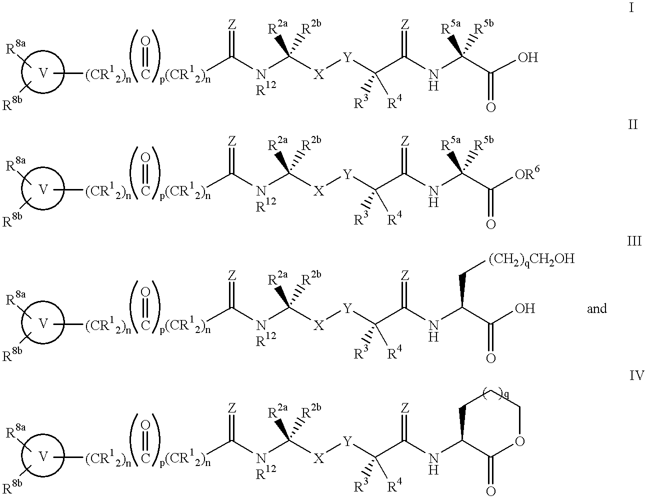

wherein in said forrnulae I, II, III and IV:

R¹ is hydrogen, C₁-C₆ alkyl or aryl;

R^{2a} and R^{2b} are independently selected from:

a) a side chain of a naturally occurring amino acid,

b) an oxidized forrn of a side chain of a naturally occurring amino acid which is:

i) methionine sulfoxide, or

ii) methionine sulfone,

c) substituted or unsubstituted C₁-C₂₀ alkyl, C₂-C₂₀ alkenyl, C₃-C₁₀ cycloalkyl, aryl or heterocycle group, wherein the substituent is selected from F, Cl, Br, NO₂, R⁹0-, R¹⁰S(O)ₘ—, R⁹C(O)NR⁹-, CN, (R⁹)₂NC(NR⁹)—, R⁹C(O)—, R⁹OC(O)—, N₃, —N(R⁹)₂, R¹⁰OC(O)NR⁹- and C₁-C₂₀ alkyl, and

d) C₁-C₆ alkyl substituted with an unsubstituted or substituted group selected from aryl, heterocycle and C₃-C₁₀ cycloalkyl; or

R^{2a} and R^{2b} are combined to form —(CH₂)ₛ—;

R³ and R⁴ are independently selected from:

a) a side chain of a naturally occurring amino acid,

b) an oxidized form of a side chain of a naturally occurring amino acid which is:

i) methionine sulfoxide, or

ii) methionine sulfone,

c) substituted or unsubstituted C₁-C₂₀ alkyl, C₂-C₂₀ alkenyl, C₃-C₁₀ cycloalkyl, aryl or heterocycle group, wherein the substituent is selected from F, Cl, Br, NO₂, R⁹0-, R¹OS(O)ₘ—, R⁹C(O)NR⁹-, CN, (R⁹)₂NCNR⁹)—, R⁹C(O)—, R⁹OC(O)—, N₃, —N(R⁹)₂, R¹⁰OC(O)NR⁹- and C₁-C₂₀ alkyl, and

d) C₁-C₆ alkyl substituted with an unsubstituted or substituted group selected from aryl, heterocycle and C₃-C₁₀ cycloalkyl; or

R³ and R⁴ are combined to form —(CH₂)ₛ—;

R^{5a} and R^{5b} are independently selected from:

a) a side chain of a naturally occurring amino acid,

b) an oxidized form of a side chain of a naturally occurring amino acid which is:

i) methionine sulfoxide, or

ii) methionine sulfone,

c) substituted or unsubstituted C₁-C₂₀ alkyl, C₂-C₂₀ alkenyl, C₃-C₁₀ cycloalkyl, aryl or heterocycle group, wherein the substituent is selected from F, Cl, Br, NO₂, R⁹0-, R¹OS(O)ₘ—, R⁹C(O)NR⁹-, CN, (R⁹)₂N—C(NR⁹)—, R⁹C(O)—, R⁹OC(O)—, N₃, —N(R⁹)₂, R¹⁰OC(O)NR⁹- and C₁-C₂₀ alkyl, and

d) C₁-C₆ alkyl substituted with an unsubstituted or substituted group selected from aryl, heterocycle and C₃-C₁₀ cycloalkyl; or

R^{5a} and R^{5b} are combined to form —(CH₂)ₛ— wherein one of the carbon atoms is optionally replaced by a moiety selected from O, S(O)ₘ, —NC(O)—, and —N(COR⁹)—;

R⁶ is

a) substituted or unsubstituted C₁-C₈ alkyl, wherein the substituent on the alkyl is selected from:

1) aryl,

2) heterocycle,

3) —N(R¹⁰)₂,

4) —OR⁹, or

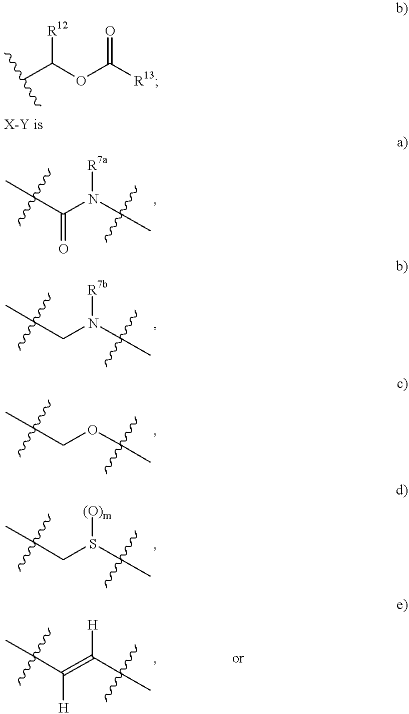

f) —CH₂—CH₂—;

R^{7a} is selected from

a) hydrogen,

b) unsubstituted or substituted aryl,

c) unsubstituted or substituted heterocycle,

d) unsubstituted or substituted cycloalkyl, and

e) C₁-C₆ alkyl substituted with hydrogen or an unsubstituted or substituted group selected from aryl, heterocycle and cycloalkyl;

R^{7b} is selected from

a) hydrogen,

b) unsubstituted or substituted aryl,

c) unsubstituted or substituted heterocycle,

d) unsubstituted or substituted cycloalkyl,

e) C₁-C₆ alkyl substituted with hydrogen or an unsubstituted or substituted group selected from aryl, heterocycle and cycloalkyl,

f) a carbonyl group which is bonded to an unsubstituted or substituted group selected from aryl, heterocycle, cycloalkyl and C₁-C₆ alkyl substituted with hydrogen or an unsubstituted or substituted group selected from aryl, heterocycle and cycloalkyl, and

g) a sulfonyl group which is bonded to an unsubstituted or substituted group selected from aryl, heterocycle, cycloalkyl and C₁-C₆ alkyl substituted with hydrogen or an unsubstituted or substituted group selected from aryl, heterocycle and cycloalkyl;

R^{8a} and R^{8b} are independently selected from hydrogen, F, Cl, Br, NO₂, R¹¹O—, R¹⁰S(O)ₘ—, CN, R⁹C(O)NR⁹-, (R⁹)₂N—C(NR⁹)—, R⁹C(O)—, R⁹OC(O)—, N₃, —N(R⁹)₂, R¹⁰OC(O)NR⁹-, C₁-C₂₀ alkyl, aryl, heterocycle or C₁-C₂₀ alkyl substituted with aryl or heterocycle;

R⁹ is independently selected from hydrogen, C₁-C₆ alkyl and aryl;

R¹⁰ is independently selected from C₁-C₆ alkyl and aryl;

R¹¹ is independently selected from hydrogen, C₁-C₆ alkyl and aryl, provided R¹¹ is C₁-C₆ alkyl when n is 0:

R¹² is independently hydrogen or C₁-C₆ alkyl;

R¹³ is C₁-C₆ alkyl;

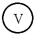

is aryl or 1,2,3,4-tetrahydronaphthyl;

Z is independently H₂ or O;

m is 0, 1 or 2;

n is independently 0 to 4;

p is 0 or 1;

q is 0, 1 or 2; and

s is 4 or 5;

(t) compounds of the following formulae, which compounds are also disclosed in PCT Publication 96/10034, incorporated herein by reference,

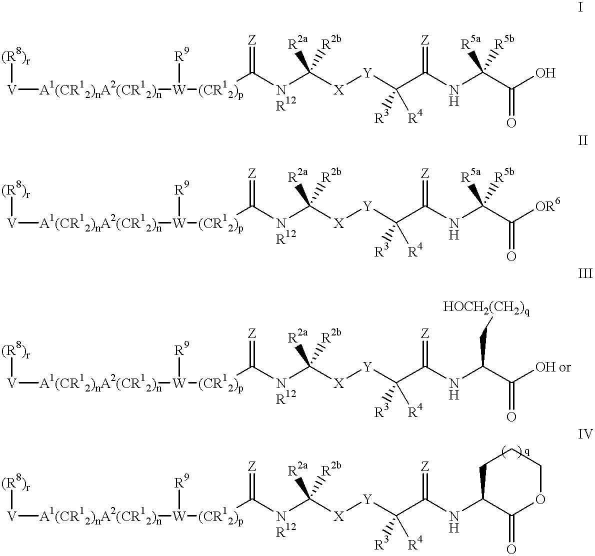

wherein in said formulae I, II, III and IV:

R¹ is independently selected from:

a) hydrogen,

b) aryl, heterocyclic, cycloalkyl, alkenyl, alkynyl, R¹¹OR¹¹S(O)ₘ—, R¹¹C(O)NR¹⁰-, CN, NO₂, (R¹⁰)₂NC(NR¹⁰)—, R¹⁰C(O)—, R¹⁰OC(O)—, N₃, —N(R¹¹)₂, or R¹¹OC(O)NR¹⁰

c) C₁-C₆ alkyl unsubstituted or substituted by aryl, heterocyclic, cycloalkyl, alkenyl, alkynyl, R¹⁰O—, R¹¹S(O)ₘ—, R¹⁰C(O)NR¹⁰-, CN, (R¹⁰)₂N—C(NR¹⁰)—, R¹⁰C(O)—, R¹⁰OC(O)—, N₃, —N(R¹⁰)₂, or R¹¹OC(O)NR¹⁰-;

R^{2a} and R^{2b} are independently selected from:

a) a side chain of a naturally occurring amino acid,

b) an oxidized form of a side chain of a naturally occurring amino acid which is:

i) methionine sulfoxide, or

ii) methionine sulfone,

c) substituted or unsubstituted C₁-C₂₀ alkyl, C₂-C₂₀ alkenyl, C₃-C₁₀ cycloalkyl, aryl or heterocyclic group, wherein the substituent is selected from F, Cl, Br, NO₂, R¹⁰O—, R¹¹S(O)ₘ—, R¹⁰C(O)NR¹⁰-, CN, (R¹⁰)₂N—C(R¹⁰)—, R¹⁰C(O)—, R¹⁰C(O)—, N₃, —N(R¹⁰)₂, R¹¹OC(O)NR¹⁰- and C₁-C₂₀ alkyl, and

d) C₁-C₆ alkyl substituted with an unsubstituted or substituted group selected from aryl, heterocycle and C₃-C₁₀ cycloalkyl; or

R^{2a} and R^{2b} are combined to form —(CH₂)ₛ—;

R³ and R⁴ are independently selected from:

a) a side chain of a naturally occurring amino acid,

b) an oxidized form of a side chain of a naturally occurring amino acid which is:

i) methionine sulfoxide, or

ii) methionine sulfone,

c) substituted or unsubstituted C₁-C₂₀ alkyl, C₂-C₂₀ alkenyl, C₃-C₁₀ cycloalkyl, aryl or heterocyclic group, wherein the substituent is selected from F, Cl, Br, N(R¹⁰)₂, NO₂, R¹⁰O—, R¹¹S(O)ₘ—, R¹⁰C(O)NR¹⁰-, CN, (R¹⁰)₂N—C(NR¹⁰)—, R¹⁰C(O)—, R¹⁰C(O)—, N₃, —N(R¹⁰)₂, R¹¹OC(O)NR¹⁰- and C₁-C₂₀ alkyl, and

d) C₁-C₆ alkyl substituted with an unsubstituted or substituted group selected from aryl, heterocycle and C₃-C₁₀ cycloalkyl; or

R³ and R⁴ are combined to form —(CH₂)ₛ—;

R^{5a} and R^{5b} are independently selected from:

a) a side chain of a naturally occurring amino acid,

b) an oxidized form of a side chain of a naturally occurring amino acid which is:

i) methionine sulfoxide, or

ii) methionine sulfone,

c) substituted or unsubstituted C₁-C₂₀ alkyl, C₂-C₂₀ alkenyl, C₃-C₁₀ cycloalkyl, aryl or heterocycle group, wherein the substituent is selected from F, Cl, Br, N(R¹⁰)₂, NO₂, R¹⁰O—, R¹¹S(O)ₘ—, R¹⁰C(O)NR¹⁰-, CN, (R¹⁰)₂N—C(NR¹⁰)—, R¹⁰C(O)—, R¹⁰OC(O)—, N₃, —N(R¹⁰)₂, R¹¹OC(O)NR¹⁰- and C₁-C₂₀ alkyl, and

d) C₁-C₆ alkyl substituted with an unsubstituted or substituted group selected from aryl, heterocycle and C₃-C₁₀ cycloalkyl; or

R^{5a} and R^{5b} are combined to form —(CH₂)ₛ—wherein one of the carbon atoms is optionally replaced by a moiety selected from O, S(O)ₘ, —NC(O)—, and —N(COR¹)—;

R⁶ is

a) substituted or unsubstituted C₁-C₈ alkyl, wherein the substituent on the alkyl is selected from:

1) aryl,

2) heterocycle,

3) —N(R¹¹)₂,

4) —OR¹⁰. or

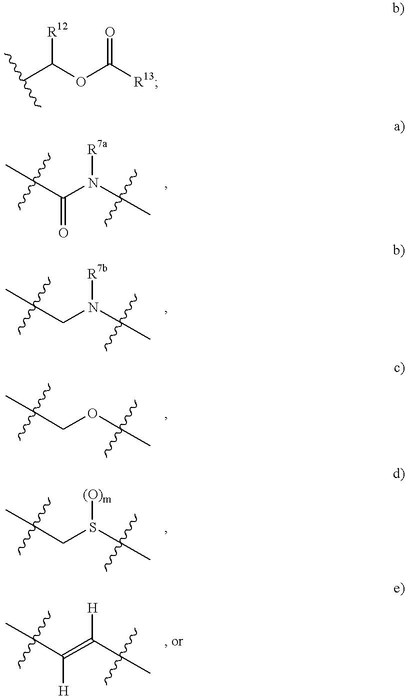

f) —CH₂—CH₂—;

R^{7a} is selected from

a) hydrogen,

b) unsubstituted or substituted aryl,

c) unsubstituted or substituted heterocyclic,

d) unsubstituted or substituted cycloalkyl, and

e) C₁-C₆ alkyl substituted with hydrogen or an unsubstituted or substituted group selected from aryl, heterocyclic and cycloalkyl;

R^{7b} is selected from

a) hydrogen,

b) unsubstituted or substituted aryl,

c) unsubstituted or substituted heterocyclic,

d) unsubstituted or substituted cycloalkyl,

e) C₁-C₆ alkyl substituted with hydrogen or an unsubstituted or substituted group selected from aryl, heterocyclic and cycloalkyl,

f) a carbonyl group which is bonded to an unsubstituted or substituted group selected from aryl, heterocyclic, cycloalkyl and C₁-C₆ alkyl substituted with hydrogen or an unsubstituted or substituted group selected from aryl, heterocyclic and cycloalkyl, and

g) a sulfonyl group which is bonded to an unsubstituted or substituted group selected from aryl, heterocyclic, cycloalkyl and C₁-C₆ alkyl substituted with hydrogen or an unsubstituted or substituted group selected from aryl, heterocyclic and cycloalkyl;

R⁸ is independently selected from:

a) hydrogen,

b) aryl, heterocyclic, cycloalkyl, alkenyl, alkynyl, perfluoroalkyl, F, Cl, Br, R¹⁰O—, R¹¹S(O)ₘ—, R¹¹C(O)NR¹⁰-, CN, NO₂, (R⁸)₂N—C(NR¹⁰)—, R¹⁰C(O)—, R¹⁰OC(O)—, N₃, —N(R¹⁰)₂, or R¹¹OC(O)NR¹⁰-, and

c) C₁-C₆ alkyl unsubstituted or substituted by aryl, heterocyclic, cycloalkyl, alkenyl, alkynyl, perfluoroalkyl, F, Cl, Br, R¹⁰O—, R¹¹S(O)ₘ—, R¹⁰C(O)NH—, CN, H₂N—C(NH)—, R¹⁰C(O)—, R¹⁰OC(O)—, N₃, —N(R¹⁰)₂, or R¹¹OC(O)NH—;

R⁹ is selected from:

a) hydrogen, alkenyl, alkynyl, perfluoroalkyl, F, Cl, Br, R¹⁰O—, R¹¹S(O)ₘ—. R¹⁰C(O)NR¹⁰-, CN, NO₂, (R¹⁰)₂NC(NR¹⁰)—, R¹⁰C(O)—, R¹⁰OC(O)—, N₃, —N(R¹⁰)₂, or R¹¹OC(O)NR¹⁰-, and

c) C₁-C₆ alkyl unsubstituted or substituted by perfluoroalkyl, F, Cl, Br, R¹⁰O—, R¹¹S(O)ₘ—, R¹⁰C(O)NR¹⁰-, CN, (R¹⁰)2N—C(NR¹⁰)—, R¹⁰C(O)—, R¹⁰OC(O)—, N₃, —N(R¹⁰)₂, or R¹¹OC(O)NR¹⁰-;

R¹⁰ is independently selected from hydrogen, C₁-C₆ alkyl and aryl;

R¹¹ is independently selected from C₁-C₆ alkyl and aryl;

R¹² is independently selected from hydrogen and C₁-C₆ alkyl;

R¹³ is independently selected from C₁-C₆ alkyl;

A₁ and A₂ are independently selected from a bond, —CH═CH—, —C≡C—, —C(O)—, —C(O)NR¹¹-, 0, —N(R¹⁰)—, —NR¹⁰C(O)—, -S(0)₂N(R¹⁰)—, —N(R¹⁰)S(O)₂— or S(O)ₘ;

V is selected from:

a) hydrogen,

b) heterocycle,

c) aryl,

d) C₁-C₂₀ alkyl wherein from 0 to 4 non-terminal carbon atoms are replaced with a heteroatom selected from O, S, and N, and

e) C₂-C₂₀ alkenyl;

provided that V is not hydrogen if A₁ is S(O)ₘ and V is not hydrogen if A₁ is a bond, n is 0 and A₂ is S(O)ₘ or a bond;

W is a heterocycle;

z is independently H₂ or O;

m is 0, 1 or 2;

n is 0, 1, 2, 3 or 4;

p is 0, 1, 2, 3 or 4;

q is 0, 1 or 2;

r is 0 to 5, provided that r is 0 when V is hydrogen; and

s is 4or 5;

(u) compounds of the following formulae, which compounds are also disclosed in PCT Publication No. WO 96/10035, incorporated herein by reference,

[chemical structure]

wherein in said formnulae I, II, III and IV:

R^{1a} and R^{1b} are independently selected from:

a) hydrogen,

b) aryl, heterocycle, C₃-C₁₀ cycloalkyl, C₂-C₆ alkenyl, C₂-C₆ alkynyl, R¹⁰O—, R¹¹S(O)ₘ—, R¹⁰C(O)NR¹⁰-, CN, NO₂, (R¹⁰)₂N—C(NR¹⁰)—, R¹⁰C(O)—, R¹⁰C(O)—, N₃, —N(R¹⁰)₂, or R¹¹OC(O)NR¹⁰-,

c) C₁-C₆ alkyl unsubstituted or substituted by aryl, heterocyclic, C₃-C₁₀cycloalkyl, C₂-C₆ alkenyl, C₂-C₆ alkynyl, R¹⁰O—, R¹¹S(O)ₘ—, R¹⁰C(O)NR¹⁰-, CN, (R¹⁰)₂N—C(NR¹⁰)—, R¹⁰C(O)—, R¹⁰OC(O)—, N₃, —N(R¹⁰)₂, or R¹¹OC(O)—NR¹⁰-;

R^{2a} and R^{2b} are independently selected from:

a) hydrogen,

b) C₁-C₆ alkyl unsubstituted or substituted by C₂-C₆ alkenyl, R¹⁰O—, R¹¹S(O)ₘ—, R¹⁰C(O)NR¹⁰-, CN, N₃, (R¹⁰)₂N—(NR¹⁰)—, R¹⁰C(O)—, R¹¹OC(O)—, —N(R¹⁰)₂, or R¹⁰C(O)NR¹⁰-,

c) aryl, heterocycle, C₃-C₁₀ cycloalkyl, C₂-C₆ alkenyl, R¹⁰O—, R¹¹S(O)ₘ—, R¹⁰C(O)NR¹⁰-, CN, NO,, (R¹⁰)₂N—C(NR¹⁰)—, R¹⁰C(O)—, R¹⁰OC(O)—, N₃, —N(R¹⁰)₂, or R¹¹OC(O)NR¹⁰-, and

d) C₁-C₆ alkyl substituted with an unsubstituted or substituted group selected from aryl, heterocyclic and C₃-C₁₀ cycloalkyl;

R³ and R⁴ are independently selected from:

a) a side chain of a naturally occurring amino acid,

b) an oxidized form of a side chain of a naturally occurring amino acid which is:

i) methionine sulfoxide, or

ii) methionine sulfone,

c) substituted or unsubstituted C₁-C₂₀ alkyl, C₂-C₂₀ alkenyl, C₃-C₁₀ cycloalkyl, aryl or heterocyclic group, wherein the substituent is selected from F, Cl, Br, N(R¹⁰)₂, NO₂, R¹⁰O—, R¹¹S(O)ₘ—, R¹⁰C(O)NR¹⁰-, CN, (R¹⁰)₂N—C(NR¹)—, R¹⁰C(O)—, R¹⁰OC(O)—, N₃, —N(R¹⁰)₂, R¹¹OC(O)NR¹⁰- and C₁-C₂₀ alkyl, and

d) C₁-C₆ alkyl substituted with an unsubstituted or substituted group selected from aryl, heterocycle and C₃-C₁₀ cycloalkyl; or

R³ and R⁴ are combined to form —(CH₂)ₛ—;

R^{5a} and R^{5b} are independently selected from:

a) a side chain of a naturally occurring amino acid,

b) an oxidized form of a side chain of a naturally occurring amino acid which is:

i) methionine sulfoxide, or

ii) methionine sulfone,

c) substituted or unsubstituted C₁-C₂₀ alkyl, C₂-C₂₀ alkenyl, C₃-C₁₀ cycloalkyl, aryl or heterocycle group, wherein the substituent is selected from F, Cl, Br, CF₃, N(R¹⁰)₂, NO₂, R¹⁰O—, R¹¹S(O)ₘ—, R¹⁰C(O)NR¹⁰-, CN, (R¹⁰)₂N—CCNR¹⁰)—, R¹⁰C(O)—, R¹⁰C(O)—, N₃, —N(R¹⁰)₂, R¹⁰C(O)N—R¹⁰- and C₁-C₂₀ alkyl, and

d) C₁-C₆ alkyl substituted with an unsubstituted or substituted group selected from aryl. heterocycle and C₃-C₁₀ cycloalkyl; or

R^{5a} and R^{5b} are combined to form —(CH₂)ₛ—wherein one of the carbon atoms is optionally replaced by a moiety selected from O, S(O)ₘ, —NC(O)—, and —N(COR¹⁰)—;

R⁶ is

a) substituted or unsubstituted C₁-C₈ alkyl, substituted or unsubstituted C₁-C₈ cycloalkyl, or substituted or unsubstituted cyclic amine, wherein the substituted alkyl, cycloalkyl or cyclic amine is substituted with 1 or 2 substituents independently selected from:

1) C₁-C₆ alkyl,

2) aryl,

3) heterocycle,

4) —N(R¹¹)₂,

5) —OR¹⁰,or

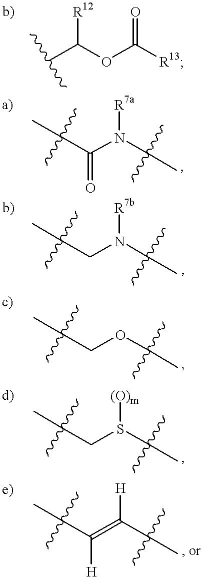

f) —CH₂—CH₂—

R^{7a} is selected from

a) hydrogen,

b) unsubstituted or substituted aryl,

c) unsubstituted or substituted heterocycle,

d) unsubstituted or substituted C₃-C₁₀ cycloalkyl, and

e) C₁-C₆ alkyl substituted with hydrogen or an unsubstituted or substituted group selected from aryl, heterocycle and C₃-C₁₀ cycloalkyl;

R^{11b} is selected from

a) hydrogen,

b) unsubstituted or substituted aryl,

c) unsubstituted or substituted heterocycle,

d) unsubstituted or substituted C₃-C₁₀ cycloalkyl,

e) C₁-C₆ alkyl substituted with hydrogen or an unsubstituted or substituted group selected from aryl, heterocycle and C₃-C₁₀ cycloalkyl,

f) a carbonyl group which is bonded to an unsubstituted or substituted group selected from aryl, heterocycle, C₃-C₁₀ cycloalkyl and C₁-C₆ alkyl substituted with hydrogen or an unsubstituted or substituted group selected from aryl, heterocycle and C₃-C₁₀ cycloalkyl, and

g) a sulfonyl group which is bonded to an unsubstituted or substituted group selected from aryl, heterocycle, C₃-C₁₀ cycloalkyl and C₁-C₆ alkyl substituted with hydrogen or an unsubstituted or substituted group selected from aryl, heterocycle and C₃-C₁₀ cycloalkyl;

R⁸⁹ is independently selected from:

a) hydrogen,

b) aryl, heterocycle, C₃-C₁₀ cycloalkyl, C₂-C₆ alkenyl, C₂-C₆ alkynyl, perfluoroalkyl, F, Cl, Br, R¹⁰O—, R¹¹S(O)ₘ—, R¹⁰C(O)NR¹⁰-, CN, NO₂, (R¹⁰)₂N—C(NR¹⁰)—R¹⁰C(O)—, R¹⁰C(O)—, N₃, —N(R¹⁰)₂, or R¹¹OC(O)NR¹⁰-, and

c) C₁-C₆ alkyl unsubstituted or substituted by aryl, heterocycle, C₃-C₁₀ cycloalkyl, C₂-C₆ alkenyl, C₂-C₆ alkynyl, perfluoroalkyl, F, Cl, Br, R¹⁰O—, R¹¹S(O)ₘ—, R¹⁰C(O)NH—, CN, H₂N—C(H)—, R¹⁰C(O)—, R¹⁰OC(O)—, N₃, —N(R¹⁰)₂, or

R¹⁰OC(O)NH—;

R⁹ is selected from:

a) hydrogen,

b) C₂-C₆ alkenyl, C₂-C₆ alkynyl, perfluoroalkyl, F, Cl, Br, R¹⁰O—, R¹¹S(O)ₘ—, R¹⁰C(O)NR¹⁰-, CN, NO₂, (R⁸)₂N—C-(NR¹⁰)—, R¹⁰C(O)—, R¹⁰OC(O)—, N₃, —N(R¹⁰)₂, or R¹¹OC(O)NR¹⁰-, and

c) C₁-C₆ alkyl unsubstituted or substituted by perfluoroalkyl, F, Cl, Br, R¹⁰O—, R¹¹S(O)ₘ—, R¹⁰C(O)NR¹⁰-, CN, (R¹⁰)₂N—C(NR¹⁰)—, R¹⁰C(O)—, R¹⁰OC(O)—, N₃, —N(R¹⁰)₂, or R¹¹OC(O)NR¹⁰;

R¹⁰ is independently selected from H, C₁-C₆ alkyl, benzyl, substituted aryl and C₁-C₆ alkyl substituted with substituted aryl;

R¹¹ is independently selected from C₁-C₆ alkyl and aryl;

R¹² is hydrogen or C₁-C₆ alkyl;

R¹³ is C₁-C₆ alkyl;

A₁ and A₂ are independently selected from a bond, —CH═CH—, —C≡C—, —C(O)—, —C(O)NR¹⁰-, —NR¹⁰C(O)—, O, —N(R¹⁰)—, —(O)₂N(R¹⁰)—, —N(R¹⁰)S(O)₂—, or S(O)ₘ;

V is selected from:

a) hydrogen,

b) heterocycle,

c) aryl,

d) C₁-C₂₀ alkyl wherein from 0 to 4 carbon atoms are replaced with a heteroatom selected from O, S, and N, and

e) C₂-C₂₀ alkenyl, provided that V is not hydrogen if A₁ is S(O)ₘ and V is not hydrogen if A₁ is a bond, n is 0 and A₂ is S(O)ₘ;

W is a heterocycle;

Z is independently H₂ or O;

m is 0, 1 or 2;

n is 0, 1, 2, 3 or 4;

p is 0, 1, 2, 3 or 4;

q is 0, 1 or 2;

r is 0 to 5, provided that r is 0 when V is hydrogen;

s is 4or 5;

t is 3, 4 or 5; and

u is 0 or 1;

(v) compounds of the following formulae,

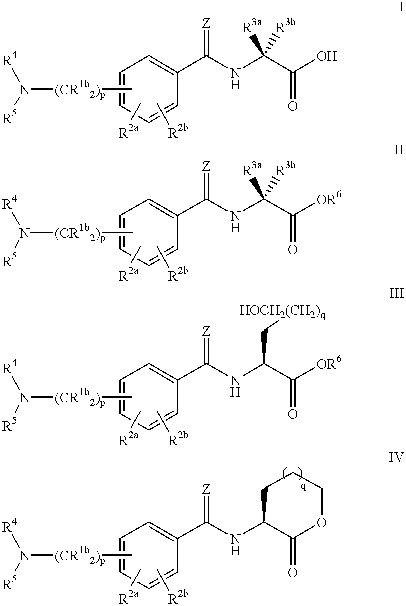

wherein in formulae I, II, III and IV:

R^{1a} and R^{1b} are independently selected from:

a) hydrogen,

b) aryl, heterocycle, cycloalkyl, alkenyl, alkynyl, R¹⁰O—, R¹¹S(O)ₘ—, R¹⁰C(O)NR¹⁰-, CN, NO₂, (R¹⁰)₂N—C(NR¹⁰)—, R¹⁰C(O)—, R¹⁰OC(O)—, N₃, —N(R¹⁰)₂, or R¹¹OC(O)NR¹⁰-,

c) C₁-C₆ alkyl unsubstituted or substituted by aryl, heterocyclic, cycloalkyl, alkenyl, alkynyl, R¹⁰O—, R¹¹S(O)ₘ—, R¹⁰C(O)NR¹⁰-, CN, (R¹⁰)₂N—C(NR¹⁰)—, R¹⁰C(O)—, R¹⁰C(O)—, N₃, —N(R¹⁰)₂, or R¹¹OC(O)—NR¹⁰-;

R^{2a} and R^{2b} are independently selected from:

a) hydrogen,

b) C₁-C₆ alkyl unsubstituted or substituted by alkenyl, R¹⁰O—, R¹¹S(O)ₘ—, R¹⁰C(O)NR¹⁰-, CN, N₃, (R¹⁰)₂N—C(NiR¹⁰)—, R¹⁰C(O)—, R¹⁰OC(O)—, —N(R¹⁰)₂, or R¹¹OC(O)NR¹⁰-,

c) aryl, heterocycle, cycloalkyl, alkenyl, R¹⁰O R¹¹S(O)ₘ—, R¹⁰C(O)NR¹⁰-, CN, NO₂, (R¹⁶)₂N—C(NR¹⁰)—, R¹⁰C(O)—, R¹⁰OC(O)—, N₃, —N(R¹⁰)₂, or R¹¹OC(O)NR¹⁰-, and

d) C₁-C₆ alkyl substituted with an unsubstituted or substituted group selected from aryl, heterocyclic and C₃-C₁₀ cycloalkyl;

R^{3a} and R^{3b} are independently selected from:

a) a side chain of a naturally occurring amino acid,

b) an oxidized form of a side chain of a naturally occurring amino acid which is:

i) methionine sulfoxide, or

ii) methionine sulfone, and

c) substituted or unsubstituted C₁-C₂₀ alkyl, C₂-C₂₀ alkenyl, C₃-C₁₀ cycloalkyl, aryl or heterocyclic group, wherein the substituent is selected from F, Cl, Br, N(R¹⁰)₂, NO₂, R¹⁰O—, R¹¹S(O)ₘ—, R¹⁰C(O)NR¹⁰-, CN, (R¹⁰)₂N—C(NR¹⁰)—, R¹⁰C(O)—, R¹⁰C(O)—, N₃, —N(R¹⁰)₂, R¹⁰OC(O)N—R¹⁰- and C₁-C₂₀ alkyl, and

d) C₁-C₆ alkyl substituted with an unsubstituted or substituted group selected from, heterocycle and C₃-C₁₀ cycloalkyl; or

R^{3a} and R^{3b} are combined to form —(CH₂)ₛ—wherein one of the carbon atoms is optionally replaced by a moiety selected from O, S(O)ₘ, —NC(O)—, and —N(COR¹⁰)—;

R⁴ and R⁵ are independently selected from:

a) hydrogen, and

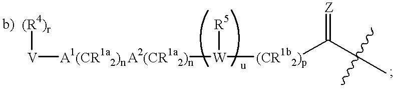

R⁶ is

a) substituted or unsubstituted C₁-C₈ alkyl or substituted or unsubstituted C₃-C₈ cycloalkyl, wherein the substituent on the alkyl is selected from:

1) aryl,

2) heterocycle,

3) —N(R¹¹)₂,

4) —OR¹⁰, or

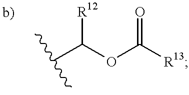

R⁷ is independently selected from:

a) hydrogen,

b) aryl, heterocycle, cycloalkyl, alkenyl, alkynyl, perfluoroalkyl, F, Cl, Br, R¹⁰O—, R¹¹S(O)ₘ—, R¹⁰C(O)NR¹⁰-, CN, NO₂, (R¹⁰)₂N—C(NR¹⁰)—, R¹⁰C(O)—, R¹⁰OC(O)—, N₃, —N(R¹⁰)₂, or R¹¹OC(O)NR¹⁰-, and

c) C₁-C₆ alkyl unsubstituted or substituted by aryl, heterocycle, cycloalkyl, alkenyl, alkynyl, perfluoroalkyl, F, Cl, Br, R¹⁰O—, R¹¹S(O)ₘ—, R¹⁰C(O)NH—, CN, H₂NC(NH)—, R¹⁰C(O)—, R¹⁰OC(O)—, N₃, —N(R¹⁰)₂, or R¹⁰OC(O)NH—;

R⁸ is selected from:

a) hydrogen,

b) alkenyl, alkynyl, perfluoroalkyl, F, Cl, Br, R¹⁰O—, R¹¹S(O)ₘ—, R¹⁰C(O)NR¹⁰-, CN, NO₂, (R¹⁰)₂N—C-(NR¹⁰)—, R¹⁰C(O)—, R¹⁰OC(O)—, N₃, —N(R¹⁰)₂, or R¹¹OC(O)NR¹⁰-, and

c) C₁-C₆ alkyl unsubstituted or substituted by perfluoroalkyl, F, Cl, Br, R¹⁰O—, R¹¹S(O)ₘ—, R¹⁰C(O)NR¹⁰-, CN, (R¹⁰)₂N—C(NR¹⁰)—, R¹⁰C(O)—, R¹⁰OC(O)—, N₃, —N(R¹⁰)₂, or R¹¹OC(O)NR¹⁰-;

R¹⁰ is independently selected from hydrogen, C₁-C₆ alkyl, benzyl and aryl;

R¹¹ is independently selected from C₁-C₆ alkyl and aryl;

R¹² is independently selected from hydrogen and C₁-C₆ alkyl; R¹³ is independently selected from C₁-C₆ alkyl;

A¹ and A² are independently selected from a bond, —CH═CH—, —C≡C—, —C(O)—, —C(O)NR¹⁰-, —NR¹⁰C(O)—, 0,—N(R¹⁰)—, -S(O)₂N(R¹⁰)—, —N(R¹⁰)S(O)₂-, or S(O)ₘ;

V is selected from:

a) hydrogen,

b) heterocycle,

c) aryl,

d) C₁-C₂₀ alkyl wherein from 0 to 4 carbon atoms are replaced with a heteroatom selected from O, S, and N, and

e) C₂-C₂₀ alkenyl, provided that V is not hydrogen if A¹ is S(O)ₘ and V is not hydrogen if A¹ is a bond, n is 0 and A² is S(O)ₘ;

W is a heterocycle;

Z is independently H₂ or O;

m is 0, 1 or 2;

n is 0, 1, 2, 3 or 4;

p is 0, 1, 2, 3 or 4;

q is 0, 1 or 2;

r is 0 to 5, provided that r is 0 when V is hydrogen;

s is 4 or 5; and

u is 0 or 1;

(w) compounds of the following formulae,

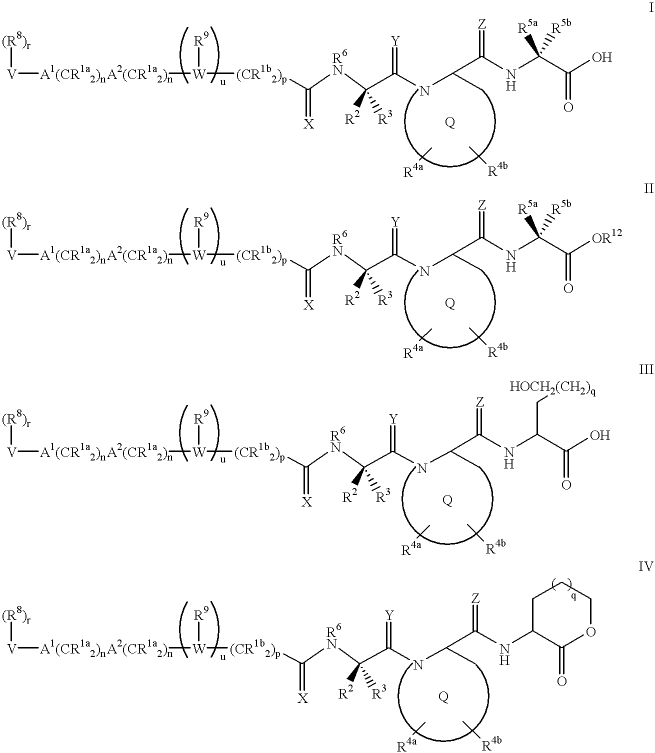

wherein in said formulae I, II, III and IV:

R^{1a} and R^{1b} are independently selected from:

a) hydrogen,

b) aryl, heterocycle, cycloalkyl, alkenyl, alkynyl, R¹⁰O—, R¹¹S(O)ₘ—, R¹⁰C(O)NR¹⁰-, CN, NO₂, (R¹⁰)₂N—C(NR¹⁰)—, R¹⁰C(O)—, R¹⁰OC(O)—, N₃, —N(R¹⁰)₂, or R¹¹OC(O)NR¹⁰-,

c) C₁-C₆ alkyl unsubstituted or substituted by aryl, heterocyclic, cycloalkyl, alkenyl, alkynyl, R¹⁰O—, R¹¹S(O)ₘ—, R¹⁰C(O)NR¹⁰-, CN, (R¹⁰)₂N—C(NR¹⁰)—, R¹⁰C(O)—, R¹⁰C(O)—, N₃, N(R¹⁰)21 or R¹¹OC(O)—NR¹⁰-;

R² and R¹ are independently selected from:

a) a side chain of a naturally occurring amino acid,

b) an oxidized form of a side chain of a naturally occurring amino acid which is:

i) methionine sulfoxide, or

ii) methionine sulfone. and

c) substituted or unsubstituted C₁-C₂₀ alkyl. C₁-C₂₀ alkenyl, C₃-C₁₀ cycloalkyl, aryl or heterocyclic group, wherein the substituent is selected from F, Cl, Br, N(R¹⁰)₂, NO,, R¹⁰O—, R¹¹S(O)ₘ—, R¹⁰C(O)NR¹⁰-, CN, (R¹⁰)₂N—C(NR¹⁰)—, R¹¹C(O)—, R¹⁰C(O)—, N₃, —N(R¹⁰)₂, R¹¹OC(O)NR¹⁰- and C₁-C₂₀ alkyl, and

d) C₁-C₆ alkyl substituted with an unsubstituted or substituted group selected from aryl, heterocycle and C₃-C₁₀ cycloalkyl; or R² and R³ are combined to form —(CH₂)ₛ—; or R² or R¹ are combined with R⁶ to form a ring such that

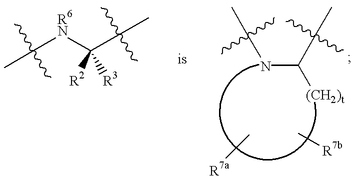

R^{4a}, R^{4b}, R^{7a} and R^{7b} are independently selected from:

a) hydrogen,

b) C₁-C₆ alkyl unsubstituted or substituted by alkenyl, R¹⁰O—, R¹¹S(O)ₘ—, R¹⁰C(O)NR¹⁰-, CN, N₃, (R¹⁰)₂N—C(NR¹⁰)—, R¹⁰C(O)—, R¹⁰OC(O)—, —N(R¹⁰)₂, or R¹¹OC(O)NR¹⁰-,

c) aryl, heterocycle, cycloalkyl, alkenyl, R¹⁰O—, R¹¹S(O)ₘ-, R¹⁰C(O)NR¹⁰-, CN, NO₂, (R¹⁰)₂N—C(NR¹⁰)—, R¹⁰C(O)—, R¹⁰C(O)—, N₃, —N(R¹⁰)₂, or R¹¹OC(O)NR¹⁰-, and

d) C₁-C₆ alkyl substituted with an unsubstituted or substituted group selected from aryl, heterocyclic and C₃-C₁₀ cycloalkyl;

R^{5a} and R^{5b} are independently selected from:

a) a side chain of a naturally occurring amino acid,

b) an oxidized form of a side chain of a naturally occurring amino acid which is:

i) methionine sulfoxide, or

ii) methionine sulfone,

c) substituted or unsubstituted C₁-C₂₀ alkyl, C₂-C₂₀ alkenyl, C₃-C₁₀ cycloalkyl, aryl or heterocycle group, wherein the substituent is selected from F, Cl, Br, N(R¹⁰)₂, NO₂, R¹⁰O—, R¹¹S(O)ₘ—, R¹⁰C(O)NR¹⁰-, CN, (R¹⁰)₂N—C(NR¹⁰)—, R¹⁰C(O)—, R¹⁰C(O)—, N₃, —N(R¹⁰)₂, R¹⁰C(O)NR¹⁰- and C₁-C₂₀ alkyl,

d) C₁-C₆ alkyl substituted with an unsubstituted or substituted group selected from aryl, heterocycle and C₃-C₁₀ cycloalkyl; or

R^{5a} and R^{5b} are combined to form —(CH,)ₛ—wherein one of the carbon atoms is optionally replaced by a moiety select from O, S(O)ₘ, —NC(O)—, and —N(COR¹⁰)—;′

R⁶ is independently selected from hydrogen or C₁-C₆ alkyl;

R⁸ is independently selected from:

a) hydrogen,

b) aryl, heterocycle, cycloalkyl, alkenyl, alkynyl, perfluoroalkyl, F, Cl, Br, R¹⁰O—, R¹⁰S(O)ₘ—, R¹⁰C(O)NR¹⁰-, CN, NO₂, (R¹⁰)₂N—C(NR¹⁰)—, R¹⁰C(O)—, R¹⁰OC(O)—, N₃, —N(R¹⁰)₂, or R¹¹OC(O)NR¹⁰-, and

c) C₁-C₆ alkyl unsubstituted or substituted by aryl, heterocycle, cycloalkyl, alkenyl, alkynyl, perfluoroalkyl, F, Cl, Br, R¹⁰O—, R¹⁰S(O)ₘ—, R¹⁰C(O)NH—, CN, H₂NC(NH)—, R¹⁰C(O)—, R¹⁰OC(O)—, N₃, —N(R¹⁰)₂, or R¹⁰OC(O)NH—;

R⁹ is selected from:

a) hydrogen,

b) alkenyl, alkynyl, perfluoroalkyl, F, Cl, Br, R¹⁰O—, R¹¹S(O)ₘ—, R¹⁰C(O)NR¹⁰-, CN, NO₂, (R¹⁰)₂N—C—(NR¹⁰)—, R¹⁰C(O)—, R¹⁰OC(O)—, N₃, —N(R¹⁰)₂, or R¹⁰OC(O)NR¹⁰-, and

c) C₁-C₆ alkyl unsubstituted or substituted by perfluoroalkyl, F, Cl, Br, R¹⁰O—, R¹¹S(O)ₘ—, R¹⁰C(O)NR¹⁰-, CN, (R¹⁰)₂N—C(NR¹⁰)—, R¹⁰C(O)—, R¹⁰C(O)—, N₃, —N(R¹⁰)₂, or R¹¹OC(O)NR¹⁰-;

R¹⁰ is independently selected from hydrogen, C₁-C₆ alkyl, benzyl and aryl;

R¹¹ is independently selected from C₁-C₆ alkyl and aryl; R¹² is

a) substituted or unsubstituted C₁-C₈ alkyl or substituted or unsubstituted C₃-C₈ cycloalkyl, wherein the substituent on the alkyl or cycloalkyl is selected from:

1) aryl,

2) heterocycle,

3) —N(R¹¹)₂,

4) —OR¹⁰, or

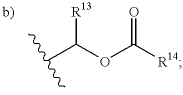

R¹³ is independently selected from hydrogen and C₁-C₆ alkyl;

R¹⁴ is independently selected from C₁-C₆ alkyl;

A₁ and A₂ are independently selected from a bond, —CH═CH—, —C≡C—, —C(O)—, —C(O)NR¹⁰-, —NR¹⁰C(O)—, O, —N(R¹⁰)—, -S(O)₂N(R¹⁰)—, —N(R¹⁰)S(O)₂—, or S(O)ₘ;

Q is a substituted or unsubstituted nitrogen-containing C₄-C₉ mono or bicyclic ring system, wherein the non-nitrogen containing ring may be an aromatic ring, a C₅-C₇ saturated ring or a heterocycle;

V is selected from:

a) hydrogen,

b) heterocycle,

c) aryl,

d) C₁-C₂₀ alkyl wherein from 0 to 4 carbon atoms are replaced with a heteroatom selected from O, S. and N, and

e) C₂-C₂₀ alkenyl, provided that V is not hydrogen if A₁ is S(O)ₘ and V is not hydrogen if A₁ is a bond, n is 0 and A₂ is S(O)ₘ;

W is a heterocycle;

X, Y and Z are independently H₂ or O;

m is 0, 1 or 2;

n is 0, 1, 2, 3 or 4;

p is 0, 1, 2, 3 or 4;

qis 0, 1 or 2;

r is 0 to 5, provided that r is 0 when V is hydrogen;

s is 4 or 5;

t is 3, 4 or 5; and

u is 0 or 1;

(x) compounds of the following formulae,

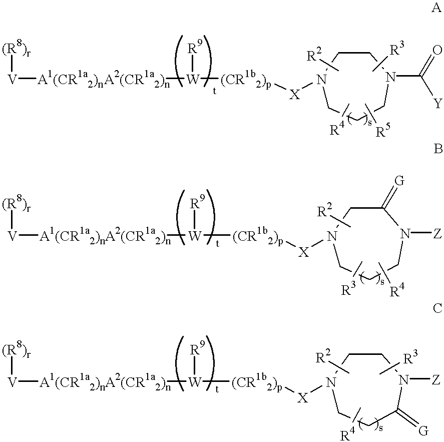

wherein in said formulae A, B and C:

R^{1a} and R^{1b} are independently selected from:

a) hydrogen,

b) aryl, heterocycle, C₃-C₁₀ cycloalkyl, C₂-C₆ alkenyl, C₂-C₆ alkynyl, R¹⁰O—, R¹¹S(O)ₘ—, R¹⁰C(O)NR¹⁰-, CN, NO₂, (R¹⁰)₂N—C(O)—, R¹⁰C(O)—, R¹⁰OC(O)—, N₃—, —N(RIO)₂, or R¹¹OC(O)NR¹⁰-,

c) C₁-C₆ alkyl unsubstituted or substituted by aryl, heterocyclic, C₃-C₁₀ cycloalkyl, C₂-C₆ alkenyl, C₂-C₆ alkynyl, R¹⁰O—, R¹¹S(O)ₘ—, R¹⁰C(O)NR¹⁰-, CN, (R¹⁰)₂N—C(NR¹⁰)—, R¹⁰C(O)—, R¹⁰OC(O)—, N₃, —N(R¹⁰)₂, or R¹¹OC(O)—NR¹⁰-;

R² and R³ are independently selected from H; unsubstituted or substituted C,., alkyl, unsubstituted or substituted C₂₋₈ alkenyl, unsubstituted or substituted C₂,₈ alkynyl, unsubstituted or substituted aryl, unsubstituted or

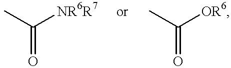

substituted heterocycle, wherein the substituted group is substituted with one or more of:

1) aryl or heterocycle, unsubstituted or substituted with:

a) C, alkyl,

b) (CH₂)pOR⁶,

c) (CH₂)pNR⁶R¹,

d) halogen,

2) C₃₋₆ cycloalkyl,

3) OR⁶,

4) SR⁶, S(O)R⁶, SO₂R⁶,

5) —NR⁶R⁷

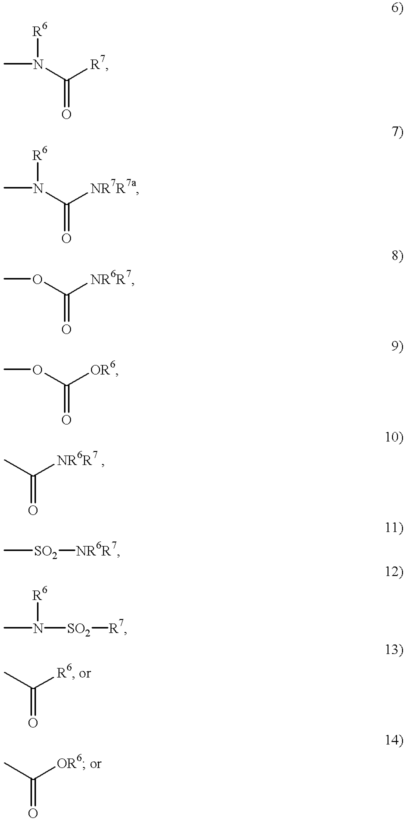

R² and R³ are attached to the same C atom and are combined to form (CH₂)u- wherein one of the carbon atoms is optionally replaced by a moiety selected from 0, S(O)ₘ, —NC(O)—, and —N(COR¹⁰)—;

R⁴ is selected from H and CH₃; and any two of R², R³ and R⁴ are optionally attached to the same carbon atom;

R⁶, R⁷ and R⁷ are independently selected from H, C₁₋₄ alkyl, C₃₋₆ cycloalkyl, heterocycle, aryl, aroyl, heteroaroyl, arylsulfonyl, heteroarylsulfonyl, unsubstituted or substituted with:

a) C₁₄ alkoxy,

b) aryl or heterocycle,

c) halogen,

d) HO,

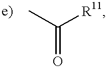

f) —SO₂R¹¹, or

g) N(R¹⁰)₂;or

R⁶ and R¹ may be joined in a ring; R⁷ and R^{7a} may be joined in a ring;

R⁸ is independently selected from:

a) hydrogen,

b) aryl, heterocycle, C₃-C₁₀ cycloalkyl, C₂-C₆ alkenyl, C₂-C₆ alkynyl, perfluoroalkyl, F, Cl, Br, R¹⁰O—, R¹¹S(O)ₘ—, R¹⁰C(O)NR¹⁰-, CN, NO₂, (R¹⁰)₂N—C(NR¹⁰)—R¹⁰C(O)—, R¹⁰OC(O)—, N₃, —N(R¹⁰)₂, or R¹⁰C(O)NR¹⁰-, and

c) C₁-C₆ alkyl unsubstituted or substituted by aryl, heterocycle, C₃-C₁₀ cycloalkyl, C₂-C₆ alkenyl, C₂-C₆ alkynyl, perfluoroalkyl, F, Cl, Br, R¹⁰O—, R¹¹S(O)ₘ— R¹⁰C(O)NH—, CN, H₂N—C(NH)—, R¹⁰C(O)—, R¹⁰OC(O)—, N₃, —N(R¹⁰)₂, or R¹⁰OC(O)N—H—;

R⁹ is selected from:

a) hydrogen,

b) alkenyl, alkynyl, perfluoroalkyl, F, Cl, Br, R¹⁰O—, R¹¹S(O)ₘ—, R¹⁰C(O)NR¹⁰-, CN, NO₂, (R¹⁰)₂N—C-(NR¹⁰)—, R¹⁰C(O)—, R¹⁰OC(O)—, N₃, —N(R¹⁰)₂, or R¹¹OC(O)NR¹⁰-, and

c) C₁-C₆ alkyl unsubstituted or substituted by perfluoroalkyl, F, Cl, Br, R¹⁰O—, R¹¹S(O),,-, R¹⁰C(O)NR¹k-, CN, (R¹⁰)₂N—C(NR¹⁰)—, R¹⁰C(O)—, R¹⁰OC(O)—, N₃, —N(R¹⁰)₂, or R¹⁰C(O)NR¹⁰-;

R¹⁰ is independently selected from hydrogen, C₁-C₆ alkyl, benzyl and aryl;

R¹¹ is independently selected from C₁-C₆ alkyl and aryl;

A₁ and A₂ are independently selected from a bond, —CH═CH—, —C≡C—, —C(O)—, —C(O)NR¹⁰-, —NR¹⁰C(O)—, O, —N(R¹⁰)—, —S(O)₂N(R¹⁰)—, —N(R¹⁰)S(O)₂-, or S(O)ₘ; G is H₂ or O;

V is selected from:

a) hydrogen,

b) heterocycle,

c) aryl,

d) C₁-C₂₀ alkyl wherein from 0 to 4 carbon atoms are replaced with a heteroatom selected from O, S, and N, and

e) C₂-C₂₀ alkenyl, provided that V is not hydrogen if A₁ is S(O)ₘ and V is not hydrogen if A₁ is a bond, n is 0 and A₂ is S(O)ₘ;

W is a heterocycle;

X is —CH₂—, —C(=O)—, or —S(═O)ₘ—;

Y is aryl, heterocycle, unsubstituted or substituted with one or more of:

1) C₁₋₄ alkyl, unsubstituted or substituted with:

a) C₁₋₄ alkoxy,

b) NR⁶R⁷,

c) C₃₋₆ cycloalkyl,

d) aryl or heterocycle,

e) HO,

f) —S(O)ₘR⁶, or

g) —C(O)NR⁶R⁷,

2) aryl or heterocycle,

3) halogen,

4) OR⁶,

5) NR⁶R⁷,

6) CN,

7) NO₂, CF₃,

9) —S(O)ₘR⁶,

10) —C(O)NR⁶R, or

11) C₃-C₆ cycloalkyl;

Z is aryl, heteroaryl, arylmethyl, heteroarylmethyl, arylsulfonyl, heteroarylsulfonyl, unsubstituted or substituted with one or more of the following:

1) C₁₋₄ alkyl, unsubstituted or substituted with:

a) C₁₋₄, alkoxy,

b) Nk⁶R⁻,

c) C₃₋₆ cycloalkyl.

d) aryl or heterocycle,

e) HO,

f) —S(O)ₘR⁶, or

g) —C(O)NR⁶R¹,

2) aryl or heterocycle,

3) halogen,

4) OR,

5) NR⁶R⁷,

6) CN,

7) NO₂

8) CF₃;

9) —S(O)ₘR⁶,

10) —C(O)NR⁶R⁷, or

11) C₃-C₆ cycloalkyl;

m is 0, 1 or 2;

n is 0, 1, 2, 3 or 4;

p is 0, 1, 2, 3 or 4;

r is 0 to 5, provided that r is 0 when V is hydrogen; is 0 to 1;

t is 0 to 1; and

u is 4 or 5;

(y) compounds of the following formula,

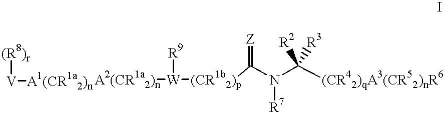

wherein:

R¹a is independently selected from:

a) hydrogen,

b) aryl, heterocycle, C₁-C₁₀ cycloalkyl, C₂-C₂₀ alkenyl, C₂-C₂₀ alkynyl, R¹⁰O—, R¹¹S(O)ₘ—, R¹⁰C(O)NR¹⁰-, CN, NO₂, (R¹⁰)₂N—C,R¹⁰)—, R¹⁰C(O)—, R¹⁰OC(O)—, N₃, —N(R¹⁰)₂, or R¹¹OC(O)NR¹⁰-,

c) C₁-C₆ alkyl unsubstituted or substituted by aryl, heterocyclic, C₃-C₁₀ cycloalkyl, C₂-C₂₀ alkenyl, C₂-C₂₀ alkynyl, R¹⁰O—, R¹¹S(O)ₘ—, R¹⁰C(O)NR¹⁰-, CN, (R¹⁰)₂N—C(NR¹⁰)—, R¹⁰C(O)—, R¹⁰C(O)—, N₃, —N(R¹⁰)₂, or R¹¹OC(O)—NR¹⁰-;

R^{1b} is independently selected from:

a) hydrogen,

b) substituted or unsubstituted aryl, substituted or unsubstituted heterocycle, C₃-C₁₀ cycloalkyl, C₂-C₂₀ alkenyl, C₂-C₂₀ alkynyl, R¹⁰O—, R¹¹S(O)ₘ—, CN, NO₂, (R¹⁰)₂N—C(NR¹⁰)—, R¹⁰C(O)—, R¹⁰OC(O)—, N₃ or —N(R¹⁰)₂,

c) C₁-C₆ alkyl unsubstituted or substituted by substituted or unsubstituted aryl, substituted or unsubstituted heterocyclic, C₃-C₁₀ cycloalkyl, C₂-C₂₀ alkenyl, C₂-C₂₀ alkynyl, R¹⁰O—, R¹¹S(O)ₘ—, CN, (R¹⁰)₂N—C(NR⁰)—, R¹⁰C(O)—, R¹⁰OC(O)—, N₃ or —N(R¹⁰)₂;

R² and R³ are independently selected from:

a) a side chain of a naturally occurring amino acid,

b) an oxidized form of a side chain of a naturally occurring amino acid which is:

i) methionine sulfoxide, or

ii) methionine sulfone, and

c) substituted or unsubstituted C₁-C₂₀ alkyl, substituted or unsubstituted C₂-C₂₀ alkenyl, substituted or unsubstituted C₃-C₁₀ cycloalkyl, substituted or unsubstituted aryl or substituted or unsubstituted heterocyclic group, wherein the substituent is selected from F, Cl, Br, N(R¹⁰)₂, NO₂, R¹⁰O—, R¹¹S(O)ₘ—, R¹⁰C(O)NR¹⁰-, CN, (R¹⁰)₂N—C(NR¹⁰)—, R⁸C(O)—, R¹⁰OC(O)—, N₃, —N(R¹⁰)₂, R¹¹OC(O)NR¹⁰- and C₁-C₂₀ alkyl, and

d) C₁-C₆ alkyl substituted with an unsubstituted or substituted group selected from aryl, heterocycle and C₃-C₁₀ cycloalkyl; or

R² and R³ are combined to form —(CH₂)ₛ—; or

R² or R³ are combined with R⁷ to formn a ring such that

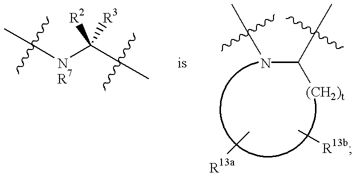

R⁴, R⁵, R^{13a} and R^{13b} are independently selected from:

a) hydrogen,

b) C₁-C₆ alkyl unsubstituted or substituted by C₂-C₂₀ alkenyl, R¹⁰O—, R¹¹S(O)ₘ—, R¹⁰C(O)NR¹⁰-, CN, N₃, (R⁸)₂N—C(NR¹⁰)—, R¹⁰C(O)—, —N(R¹¹)₂, or R¹¹OC(O)NR¹⁰-,

c) unsubstituted or substituted aryl, unsubstituted or substituted heterocycle, C₃-C₁₀, cycloalkyl, C₂-C₂₀ alkenyl, R¹⁰O—, R¹¹S(O)ₘ—, R¹⁰C(O)NR¹⁰-, CN, NO₂, (R¹⁰)₂N—C(R¹⁰)—, R¹⁰C(O)—, N₃, —N(R¹⁰)₂, or R¹¹OC(O)NR¹⁰-, and

d) C₁-C₆ alkyl substituted with an unsubstituted or substituted group selected from aryl, heterocyclic and C₃-C₁₀ cycloalkyl;

R⁶ is selected from:

a) hydrogen,

b) substituted or unsubstituted aryl, substituted or unsubstituted heterocycle, C₃-C₁₀ cycloalkyl, C₂-C₂₀ alkenyl, C₂-C₂₀ alkynyl, C₁-C₂₀ perfluoroalkyl, allyloxy, F, Cl, Br, R¹⁰O—, R¹¹S(O)ₘ—, R¹⁰C(O)NR¹⁰-, CN, NO₂, R¹⁰2N—C(NR¹⁰)—, R¹⁰C(O)—, N₃, —N(R¹⁰)₂, (R¹²)₂NC(O)— or R¹¹OC(O)NR¹⁰-, and

c) C₁-C₆ alkyl unsubstituted or substituted by substituted or unsubstituted aryl, substituted or unsubstituted heterocycle, C₃-C₁₀ cycloalkyl, C₂-C₂₀ alkenyl, C₂-C₂₀ alkynyl, C₂-C₂₀ perfluoroalkyl, F, Cl, Br, R¹⁰O—, R¹¹S(O)ₘ—, R¹⁰C(O)NH—, CN, H₂N—C(H)—, R¹⁰C(O)—, N₃, —N(R¹⁰)₂, or R¹⁰OC(O)NH—;

R¹ is independently selected from

a) hydrogen,

b) unsubstituted or substituted aryl,

c) unsubstituted or substituted heterocycle,

d) unsubstituted or substituted C₃-C₁₀ cycloalkyl, and

e) C₁-C₆ alkyl substituted with hydrogen or an unsubstituted or substituted group selected from aryl, heterocycle and C₃-C₁₀ cycloalkyl;

R⁸ is selected from:

a) hydrogen,

b) substituted or unsubstituted aryl, substituted or unsubstituted heterocycle, C₃-C₁₀ cycloalkyl, C₂-C₂₀ alkenyl, C₂-C₂₀ alkynyl, C₁-C₂₀ perfluoroalkyl, allyloxy, F, Cl, Br, R¹⁰O—, R¹¹S(O)ₘ—, R¹⁰C(O)NR¹⁰-, —S(O)₂N(R¹⁰)₂, CN, NO₂, (R¹⁰)₂N—C(NR¹⁰)—, R¹⁰C(O)—, R¹⁰OC(O)—, N₃, —N(R¹⁰)₂, or R¹¹OC(O)NR¹⁰-, and

c) C₁-C₆ alkyl unsubstituted or substituted by substituted or unsubstituted aryl, substituted or unsubstituted heterocycle, C₃-Cl, cycloalkyl, C₂-C₂₀ alkenyl, C₂-C₂₀ alkynyl, C₂-C₂₀ perfluoroalkyl, F, Cl, Br, R¹⁰O—, R¹¹S(O)ₘ—, R¹⁰C(O)NH—, CN, H₂N—C(NH)—, R¹⁰C(O)—, R¹⁰OC(O)—, N₃, —N(R¹⁰)₂1 or R¹⁰OC(O)NH—;

R⁹ is selected from:

a) hydrogen,

b) C₂-C₂₀ alkenyl, C₂-C₂₀ alkynyl, C₂-C₂₀ perfluoroalkyl, F, Cl, Br, R¹⁰O—, R¹¹S(O)ₘ—, R¹⁰C(O)NR¹⁰-, CN, NO₂, (R¹⁰)₂N—C-(NR¹⁰)—, R¹⁰C(O)—, R¹⁰OC(O)—, N₃, —N(R¹⁰)₂, or R¹¹OC(O)NR¹⁰-, and

c) C₁-C₆ alkyl unsubstituted or substituted by C₂-C₂₀ perfluoroalkyl, F, Cl, Br, R¹⁰O—, R¹¹S(O)ₘ—, R¹⁰C(O)NR¹⁰-, CN, (R⁸)₂N—CiR¹⁰)—, R¹⁰C(O)—, R¹⁰OC(O)—, N₃, —N(R¹⁰)₂, or R¹¹OC(O)NR¹⁰-;

R¹⁰ is independently selected from hydrogen, C₁-C₆ alkyl, benzyl and aryl;

R¹¹ is independently selected from C₁-C₆ alkyl and aryl;

R¹² is independently selected from hydrogen, C₁-C₆ alkyl and aryl, or (R¹

2)₂ forms —(CH₂)ₛ—;

A₁, A₂ and A₃ are independently selected from a bond, —CH═CH—, —C≡C—, —C(O)—, —C(O)NR⁷-, —NR⁷C(O)—, O, —N(R⁷)—, —S(O)₂N(R⁷)—, —N(R⁷)S(O)₂-, or S(O)ₘ;

V is selected from:

a) hydrogen,

b) heterocycle,

c) aryl,

d) C₁-C₂₀ alkyl wherein from 0 to 4 carbon atoms are replaced with a heteroatom selected from O, S, and N, and

e) C₂-C₂₀ alkenyl, provided that V is not hydrogen if A₁ is S(O)ₘ and V is not hydrogen if A₁ is a bond, n is 0 and A₂ is S(O)ₘ;

W is a heterocycle;

Z is independently H₂ or O;

m is 0, 1 or 2;

n is 0, 1, 2, 3 or 4;

p is 0, 1, 2, 3 or 4;

q is 0, 1, 2, 3 or 4;

r is 0 to 5, provided that r is 0 when V is hydrogen;

s is 4or 5; and

t is 3,4 or 5; and

(z) compounds of the following formula,

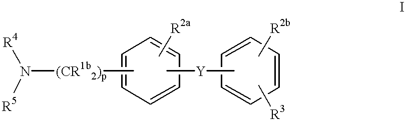

wherein:

R^{1a} and R^{1b} are independently selected from:

a) hydrogen,

b) unsubstituted or substituted aryl, unsubstituted or substituted heterocycle, unsubstituted or substituted C₃-C₆ cycloalkyl, C₂-C₆ alkenyl, C₂-C₆ alkynyl, R⁸O—, R⁹S(O)ₘ—, R⁸C(O)NR⁸-, CN, NO₂, (R⁸)₂N—C(NR⁸)—, R⁸C(O)—, R⁸OC(O)—, N₃, —N(R⁸)₂, or R⁹OC(O)N⁸-,

c) C₁-C₆ alkyl unsubstituted or substituted by unsubstituted or substituted aryl, unsubstituted or substituted heterocyclic, unsubstituted or substituted C₃-C₆ cycloalkyl, C₂-C₆ alkenyl, C₂-C₆ alkynyl, R⁸0-, R⁹S(O)ₘ—, R⁸C(O)NR¹⁰-, CN, (R⁸)₂N—C(NR⁸)—, R⁸C(O)—, R⁸OC(O)—, N₃, —N(R⁸)₂, or R⁹OC(O)—NR⁸-;

R^{2a}, R^{2b} and R³ are independently selected from:

a) hydrogen,

b) C₁-C₆ alkyl unsubstituted or substituted by C₂-C₆ alkenyl, R⁸O—, R⁹S(O)ₘ—, R⁸C(O)N⁸-, CN, N₃, (R⁸)₂N—C(NR⁸)—, R⁸C(O)—, R⁸OC(O)—, —N(R⁸)₂, or R⁹OC(O)NR⁸-,

c) unsubstituted or substituted aryl, unsubstituted or substituted heterocycle, unsubstituted or substituted cycloalkyl, alkenyl, R⁸O—, R⁹S(O)ₘ—, R⁸C(O)NR¹⁰-, CN, NO₂, (R⁸)₂N—C(NR⁸)—, R⁸C(O)—, R⁸OC(O)—, N₃, —N(R⁸)₂, halogen or R⁹OC(O)Nr⁸-, and

d) C₁-C₆ alkyl substituted with an unsubstituted or substituted group selected from aryl, heterocyclic and C₃-C₁₀ cycloalkyl;

R⁴ and R⁵ are independently selected from:

a) hydrogen, and

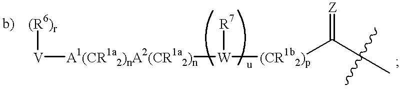

R⁶ is independently selected from:

a) hydrogen,

b) unsubstituted or substituted aryl, unsubstituted or substituted heterocycle, unsubstituted or substituted C₃-C₆ cycloalkyl, C₂-C₆ alkenyl, C₂-C₆ alkynyl, C₁-C₆ perfluoroalkyl, F, Cl, is Br, R⁸O—, R⁹S(O)ₘ—, R⁸C(O)NR⁸-, CN, NO₂, (R⁸)₂NC(NR⁸)—, R⁸C(O)—, R⁸OC(O)—, N₃, —N(R⁸)₂, or R⁹OC(O)N⁸-, and

c) C₁-C₆ alkyl unsubstituted or substituted by unsubstituted or substituted aryl, unsubstituted or substituted heterocycle, unsubstituted or substituted C₃-C₆ cycloalkyl, C₂-C₆ alkenyl, C₂-C₆ alkynyl, C₁-C₆ perfluoroalkyl, F, Cl, Br, R⁸O—, R⁹S(O)ₘ—, R⁸C(O)NH—, CN, H₂N—C(H)—, R⁸C(O)—, R⁸OC(O)—, N₃, —N(R⁸)₂, or R¹⁰C(O)NH—;

R is selected from:

a) hydrogen,

b) C₂-C₆ alkenyl, C₂-C₆ alkynyl, C₁-C₆ perfluoroalkyl, F, Cl, Br, R⁸O—, R⁹S(O)ₘ—, R⁸C(O)NR¹⁰-, CN, NO₂, (R⁸)₂N—C(NR⁸)—, R⁸C(O)—, R⁸OC(O)—, N₃, —N(R⁸)₂, or R⁹OC(O)NR⁸-, and

c) C₁-C₆ alkyl unsubstituted or substituted by C₁-C₆ perfluoroalkyl, F, Cl, Br, R⁸O—, R⁹S(O)ₘ—, R⁸C(O)NR¹¹S—, CN, (R⁸)₂N—C(NR⁸)—, R⁸C(O)—, R⁸OC(O)—, N₃—N(R⁸)₂, or R⁹OC(O)NR⁸-;

R⁸ is independently selected from hydrogen, C₁-C₆ alkyl, substituted or unsubstituted C₁-C₆ aralkyl and substituted or unsubstituted aryl;

R⁹ is independently selected from C₁-C₆ alkyl and aryl;

R¹⁰ is independently selected from hydrogen, C₁-C₆ alkyl, substituted or unsubstituted C₁-C₆ aralkyl and substituted or unsubstituted aryl;

A₁ and A₂ are independently selected from a bond, —CH═CH—, —C≡C—, is —C(O)—, —C(O)NR⁸-, —NR⁸C(O)—, O, —N(R⁸)—, —S(O)₂N(R⁸)—, —N(R⁸)S(O)₂—, or S(O)ₘ;

V is selected from:

a) hydrogen,

b) heterocycle,

c) aryl,

d) C₁-C₂₀ alkyl wherein from 0 to 4 carbon atoms are replaced with a heteroatom selected from O, S, and N, and

e) C₂-C₂₀ alkenyl, provided that V is not hydrogen if A₁ is S(O)ₘ and V is not hydrogen if

A₁ is a bond, n is 0 and A₂ is S(O)ₘ;

W is a heterocycle;

Y is selected from a bond, —C(R¹⁰)═C(R¹⁰)—, —C≡C—, —C(O)—, —C(R¹⁰)₂—, —C(OR¹⁰)R¹⁰-, —CN(R¹⁰)₂R¹⁰-, —OC(R¹⁰)₂-, —NR¹⁰C(R¹⁰)₂-, —C(R⁸)20-, —C(R¹⁰)₂NR¹⁰, —C(O)NR¹⁰-, —NR¹⁰C(O)—, O, —NC(O)R¹⁰-, —NC(O)OR¹⁰-, —S(O)₂N(R¹⁰)—, —N(R¹⁰)s(O)₂—, or S(O)ₘ;

Z is H₂or O;

m is 0, or 2;

nis 0, 1, 2, or 4;

p is 0, 1, 2, 3 or 4;

r is 0 to 5, provided that r is 0 when V is hydrogen; and u is 0 or 1;

or the pharmaceutically acceptable salts thereof.

Compounds for use in the methods of the invention also may obtained by fermentation of cultures of novel organisms, such as the compounds disclosed in U.S. Pat. No. 5,420,334. Other suitable compounds are disclosed in U.S. Pat. No. 5,420,245; European Patent Publication No. 0618 221; PCT Patent Publication Nos. WO 94/26723; WO 95/10514; WO 95/10515; WO 95/10516; WO 95/08542; WO 95/11917; and WO 95/12612. In certain embodiments of the invention, manomycin is less preferred and may be excluded from preferred aspects of the invention.

Specifically suitable compounds include the following:

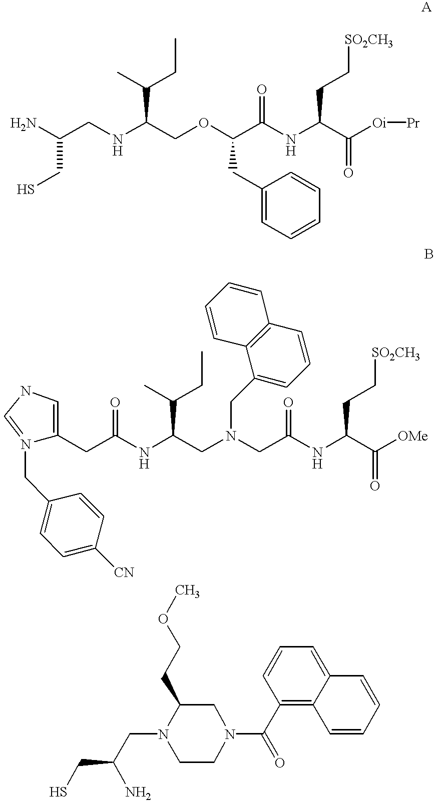

or the pharmaceutically acceptable salts thereof.

Other specifically suitable compounds include the following:

5(S)-[2(R)-amino-3-mercaptopropylamino]-6(S)-methyl-2(R)-n-propyl-3,4-E-octenoyl-homoserine, and the corresponding homoserine lactone,

5(S)-[2(R)-amino-3-mercaptopropylamino]-6(S)-methyl-2(R)-methyl-3,4-E-octenoyl-homoserine, and the corresponding homoserine lactone,

5(S)-[2(R)-amino-3-mercaptopropylamino]-6(S)-methyl-2(R)-E-octenoyl-homoserine, and the corresponding homoserine lactone,

5(S)-[2(R)-amino-3-mercaptopropylamino] -6(S)-methyl-2(R)-i-propyl-3 ,4-E-octenoyl-homoserine, and the corresponding homoserine lactone,

5(S)-[2(R)-amino-3-mercaptopropylamino]-6(S)-methyl-2(R)-n-butyl-3,4-E-octenoyl-homoserine, and the corresponding homoserine lactone,

5(S)-[2(R)-amino-3r-mercaptopropylamino]-6(S)-methyl-2(R)-s-butyl-3 ,4-E-octenoyl-homoserine, and the corresponding homoserine lactone,

5(S)-[2(R)-amino-3-mercaptopropylamino]-6(S)-methyl-2(R)-t-butyl-3,4-E-octenoyl-homoserine, and the corresponding homoserine lactone,

5(S)-[2(R)-amino-3-mercaptopropylamino]-6(S)-methyl-2(R)-cyclohexyl-3,4-E-octenoyl-homoserine, and the corresponding homoserine lactone,

5(S)-[2(R)-amino-3-mercaptopropylamino] -6(S)-methyl-2(R)-cyclopentyl-3 ,4-E-octenoyl-homoserine, and the corresponding homoserine lactone,

5(S)-[2(R)-amino-3-mercaptopropylamino]-6(S)-methyl-2(R)-benzyl-3,4-E-octenoyl-homoserine, and the corresponding homoserine lactone,

5(S)-[2(R)-amino-3-mercaptopropylamino]-6(S)-methyl-2(R)-i-propyl-3,4-E-octenoyl-homoserine, and the corresponding homoserine lactone,

5(S)-[2(R)-amino-3-mercaptopropylamino]-6(S)-2(R)-i-propyl-3 ,4-E-octenoyl-methionine, and the corresponding methyl ester,

5(S)-[2(R)-amino-3-mercaptopropylamino]-6(S)-methyl-2(R)-n-butyl-3 ,4-E-octenoyl-methionine, and the corresponding methyl ester,

5(S)-[2(R)-amino-3-mercaptopropylamino]-6(S)-methyl-2(R)-benzyl-3,4-E-octenoyl-methionine, and the corresponding methyl ester,

5(S)-[2(R)-amino-3-mercaptopropylamino]-6(S)-methyl-2(R)-n-propyl-octenoyl-homoserine, and the corresponding homoserine lactone,

5(S)-[2(R)-amino-3-mercaptopropylamino]-6(S)-methyl-2(R)-benzyl-octenoyl-homoserine, and the corresponding homoserine lactone,

N-(3-phenyl-2(S)-(mercaptopropionylamino)prop-1-yl)isoleucyl-methionine,

N-(2(R)-amino-3-mercaptopropyl)isoleucyl-phenylalanyl-methionine,

N-(3-mercaptopropyl)isoleucyl-phenylalanyl-methionine,

N-(3-mercaptopropyl)valyl-isoleucyl-methionine,

N-(2(R)-amino-3-mercaptopropyl)valyl-isoleucyl-methionine,

N-(3-methyl-2(S)-(cysteinylamino)but-1-yl)phenylalanyl-methionine,

N-(3-methyl-2(S)-(mercaptopropionylamino)butyl)-phenylalanyl-methionine,

N-[2(S)-(2(R)-amino-3-mercaptopropylamino)-3(S)methylpentyl]-phenylalanyl-methionine,

N-[2(S)-(3-mercaptopropylamino)-3-(S)methylpentyl]-phenylalanyl-methionine,

N-(2(R)-amino-3-mercaptopropyl)isoleucyl-phenylalanyl-(methionine sulfone),

N-(2(R)-amino-3-mercaptopropyl)isoleucyl)-(p-iodophenylalanyl)-methionine,

N-[2(R)-(cysteinyl-isoleucylamino)-3(S)-methylpentyl]-methionine,

N-[2(R)-(N′-(2(R)-amino-3-mercaptopropyl)—isoleucylamino)-3-phenylpropyl]methionine,

N-[2(R)-(N′-(2(R)-amino-3-mercaptopropyl)—isoleucylamino)-3(S)-methylpentyl]methionine,

N-(3-mercaptopropyl)valyl-isoleucyl-methionine methyl ester,

N-(2(R)-amino-3-mercaptopropyl)isoleucyl-phenylalanyl-methionine ethyl ester,

N-(2(R)-amino-3-mercaptopropyl)isoleucyl-phenylalanyl-methionine benzyl ester,

N-[2(R)-amino-3-mercaptopropyl]-L-isoleucine-phenethylamide,

N-[2(R)-amino-3-mercaptopropyl]-L-isoleucine-benzylamide,

N-[2(R)-amino-3-mercaptopropyl]-L-isoleucine-3-methylbutylamide,

N-[2(R)-amino-3-mercaptopropyl]-L-isoleucine-3-phenylpropylamide,

N-[2(R)-amino-3-mercaptopropyl]-L-isoleuceyl-L-phenylalaninol,

N-[2(R)-amino-3-mercaptopropyl]-L-isoleucine-N′-methylbenzylamide,

N-[2(R)-amino-3-mercaptopropyl]-L-isoleucine-(4-methoxybenzyl)amide,

N-[2(R)-amino-3-mercaptopropyl]-L-isoleucine-(2,4-dichlorobenzyl)amide,

N-[2(R)-amino-3-mercaptopropyl]-L-isoleucine-(4-trifluoromethylbenzyl)amide,

N-[2(R)-amino-3-mercaptopropyl]-L-isoleucine-(2,4-dichlorophenethyl)amino,

N-[2(R)-amino-3-mercaptopropyl]-L-isoleucine-(2-benzimidazolylmethyl)amide,

N-[2(R)-amino-3-mercaptopropyl]-L-isoleucine-(1-indanyl)amide,

N-[2(R)-amino-3-mercaptopropyl]-L-isoleucine-(2,4-dimethylbenzyl)amide,

N-[2(R)-amino-3-mercaptopropyl]-L-isoleucine-(2,3-dichlorobenzyl)amide,

N-[2(R)-amino-3-mercaptopropyl]-L-isoleucine-(4-sulfamoylbenzyl)amide,

N-[2(R)-amino-3-mercaptopropyl]-L-isoleucineanilide,

N-[2(R)-amino-3-mercaptopropyl]-L-isoleucine-(2,4-dimethylphenyl)amide,

N-[2(R)-amino-3-mercaptopropyl]-L-isoleucine-(2,3-dimethylphenyl)amide,

L-cysteinyl-L-isoleucine-phenethylamide,

N-[2(S)-[2(R)-amino-3-mercaptopropylamino]-3-methylpentyl]phenethylamide,

N-(2(R)-amino-3-mercaptopropyl)-L-alaninebenzylamide,

N-benzyl-[2(S)-2(R)-amino-3-mercaptopropyl)-amino]butyramide,

N-(2(R)-amino-3-mercaptopropyl)-L-norleucinebenzylamide,

N-(2(R)-amino-3-mercaptopropyl)-L-norvalinebenzylamide,

N-(2(R)-aino-3-mercaptopropyl)isoleucyl-phenylalanyl-homoserine,

N-(2(R)-amino-3-mercaptopropyl)isoleucyl-isoleucyl-homoserine,

N-(2(R)-a:ino-3-mercaptopropyl)isoleucyl-phenylalanyl-homoserine lactone,

N-(2(R)-amino-3-mercaptopropyl)isoleucyl-isoleucyl-homoserine lactone,

N-(2(R)-amino-3-mercaptopropyl)isoleucyl-phenylalanyl-homocysteine lactone,

N-[2(S)-(2(R)-amino-3-mercaptopropyl) -3(S)-methyl pentyl]-isoleucyl homoserine lactone,

N-[N′-(2(R)-amino-3-mercaptopropyl)isoleucyl-phenylalanyl]-3-(S)amino-tetrahydropyran-2-one,

N-[N′-(2(R)-amino-3-mercaptopropyl)isoleucyl-isoleucyl]-3-(S)-aminotetrahydropyran-2-one,

N-(2(R)-amino-3-mercaptopropyl)isoleucyl-isoleucyl-homocysteine lactone,

N-[2(S)-(2(R)-amino-3-mercaptopropyl)-3(S)-methylpentyl]isoleucyl homoserine,

N-[N′-(2(R)-amino-3-mercaptopropyl)isoleucyl-phenylalanyl]-3(S)-amino-4-hydroxypentanoic acid,

N-[N′-(2(R)-amino-3-mercaptopropyl)isoleucyl-isoleucyl]-3-(S)-amino-hydroxypentanoic acid,

N-[2(S)-(2(R)-amino-3-mercaptopropylamino)-3-(S)-methylpentyl]—N-methyl-isoleucyl-homoserine,

N-[2(S)-(2(R)-amino-3-mercaptopropylamino)-3(S)-methylpentyl]—N-methyl-isoleucyl-homoserine lactone,

N-[2(S)-(2(R)-amino-3-mercaptopropylamino)-3(S)-methylpentyl]—N-methylphenylalanyl-homoserine,

N-[2(S)-(2(R)-amino-3-mercaptopropylamino)-3(S)-methylpentyl]—N-methylphenylalanyl-homoserine lactone,

N-[2(S)-(2(R)-amino-3-mercaptopropylamino)-3-methyl-butyl]-N-methylphenylalanyl-homoserine lactone,

3(S)-(N-[2(S)-(2(R)-amino-3-mercaptopropylamino)-3-(S)-methylpentyl)-N-methyl-isoleucylamino)-3-methyltetra-hydropyran-2-one,

2(S)-(N-[2(S)-(2(R)-amino-3-mercaptopropylamino)-3(S)-methylpentyl]—N-methyl-isoleucylamino)-2-methyl-5-hydroxypentanoic acid,

2(S)-(N-[2(S)-(2(R)-amino-3-mercaptopropylamino)-3-(S)-methylpentyl]—N-methyl-isoleucylamino)-5-methyl-5-hydroxyhexanoic acid,

N-[2(S)-(2(R)-amino-3-mercaptopropylamino)-3(S)-methylpentyl]—N-methyl-norvalyl-homoserine,

N-[2(S)-(2(R)-amino-3-mercaptopropylamino)-3-(S)-methylpentyl]—N-methyl-norvalyl-homoserine lactone,

N-[2(S)-(2(R)-amino-3-mercaptopropylamino)-3-(S)-methylpentyl]—N-methyl-isoleucyl-methionine,

N-[2(S)-(2(R)-amino-3-mercaptopropylamino)-3-(S)-methylpentyl]-N-methyl-isoleucyl-methionine methyl ester,

N-[2(S)-(2(R)-amino-3-mercaptopropylamino)-3-(S)-methylpentyl]—N-methyl-phenylalanyl-methionine,

N-[2(S)-(2(R)-amino-3-mercaptopropylamino)-3(S)-methylpentyl]—N-methyl-phenylalanyl-methionine methyl ester,

3(S)-(N-[2(S)-(2(R)-amino-3-mercaptopropylamino)-3-(S)-methylpentyl]-N-methyl-isoleucylamino)-6,6-dimethyl-tetrahydropyran-2-one,

N-[2(S)-(2(R)-amino-3-mercaptopropylamino)-3-(S)-methylpentyl]—N-methyl-norvalyl-methionine,

N-[2(S)-(2(R)-amino-3-mercaptopropylamino)-3-(S)-methylpentyl]-N-methyl-norvalyl-methionine methyl ester,

N-[2(S)-(2(R)-amino-3-mercaptopropylamino)-3-(S)-methylpentyl]—N-methyl-D-norvalyl-homoserine,

N-[2(S)-(2(R)-amino-3-mercaptopropylamino)-3-(S)-methylpentyl]-N-methyl-D-norvalyl-homoserine lactone,

2(S)-[2(S)-[2(R)-amino-3-mercapto]propylamino-3(S)-methyl]-pentyloxy-3-phenylpropionyl-homoserine lactone,

2(S)-[2(S)-[2(R)-amino-3-mercapto]propylamino-3(S)-methyl]-pentyloxy-3-phenylpropionyl-homoserine,

2(S)-[2(S)-[2(R)-amino-3-mercapto]propylamino-3(S)-methyl]-pentyloxy-2-methyl-3-phenylpropionyl-homoserine lactone,

2(S)-[2(S)-[2(R)-amino-3-mercapto]propylamino-3(S)-methyl]-pentyloxy-2-methyl-3-phenylpropionyl-homoserine,

2(S)-[2(S)-[2(R)-amino-3-mercapto]propylamino-3(S)-methyl]-pentyloxy-4-pentenoyl-homoserine lactone,

2(S)-[2(S)-[2(R)-amino-3-mercapto]propylamino-3(S)-methyl]-pentyloxy-4-pentenoyl-homoserine,

2(S)-[2(S)-[2(R)-amino-3-mercapto]propylamino-3(S)-methyl]-pentyloxypentanoyl-homoserine lactone,

2(S)-[2(S)-[2(R)-amino-3-mercapto]propylamino-3(S)-methyl]-pentyloxypentanoyl-homoserine,

2(S)-[2(S)-[2(R)-amino-3-mercapto]propylamino-3(S)-methyl]-pentyloxy-4-methylpentanoyl-homoserine lactone,

2(S)-[2(S)-[2(R)-amino-3-mercapto]propylamino-3(S)-methyl]-pentyloxy-4-methylpentanoyl-homoserine,

2(S)-[2(S)-[2(R)-amino-3-mercapto]propylamino-3(S)-methyl]-pentyloxy-3-methylbutanoyl-homoserine lactone,

2(S)-[2(S)-[2(R)-amino-3-mercapto]propylamino-3(S)-methyl]-pentyloxy-3-methylbutanoyl-homoserine,

2(S)-[2(S)-[2(R)-amino-3-mercapto]propylamino-3(S)-methyl]-pentyloxy-3-phenylbutanoyl-homoserine lactone,

2(S)-[2(S)-[2(R)-amino-3-mercapto]propylanino-3(S)-methyl]-pentyloxy-3-phenylbutanoyl-homoserine,

2(S)-[2(S)-[2(R)-amino-3-mercapto]propylamino-3(S)-methyl]-pentylthio-2-methyl-3-phenylpropionyl-homoserine lactone,

2(S)-[2(S)-[2(R)-amino-3-mercapto]propylamino-3(S)-methyl]-pentylthio-2-methyl-3-phenylpropionyl-homoserine,

2(S)-[2(S)-[2(R)-amino-3-mercapto]propylamino-3(S)-methyl]-pentylsulfonyl-2-methyl-3-phenylpropionyl-homoserine lactone,

2(S)-[2(S)-[2(R)-amino-3-mercapto]propylamino-3(S)-methyl]-pentylsulfonyt-2-methyl-3-phenylpropionyl-homoserine,

2(S)-[2(S)-[2(R)-amino-3-mercapto]propylamino-3(S)-methyl]-pentyloxy-3-phenylpropionyl-methionine methyl ester,

2(S)-[2(S)-[2(R)-amino-3-mercapto]propylamino-3(S)-methyl]-pentyloxy-3-phenylpropionyl-methionine,

2(S)-[2(S)-[2 (R)-amino-3-mercapto ]propylam ino-3 (S)-methyl]-pentyloxy-3-phenylpropionyl-methionine sulfone methyl ester,

2(S)-[2(S)-[2(R)-amino-3-mercapto]propylamino-3(S)-methvl]-pentyloxy-3-phenylpropionyl-methionine sulfone,

2-(S)-[2(S)-[2(R)-amino-3-(S)-methyl]-pentyloxy-3-naphth-2-yl-propionyl-methionine sulfone methyl ester,

2-(S)-[2(S)-[2(R)-amino-3-mercapto]propylamino-3(S)-methyl]-pentyloxy-3-naphth-2-yl-propionyl-methionine sulfone,

2-(S)-[2(S)-[2(R)-amino-3-mercapto]propylamino-3-(S)-methyl]pentyloxy-3-naphth-1-yl-propionyl-methionine sulfone methyl ester,

2-(S)-[2(S)-[2(R)-amino-3-mercapto]propylamino-3(S)-methyl]-pentyloxy-3-naphth-1-yl-propionyl-methionine sulfone,

2-(S)-[2(S)-[2(R)-amino-3-mercapto]propylamino-3-(S)-methyl]pentyloxy-3-methybutanoyl-methionine methyl ester,

2-(S)-[2(S)-[2(R)-amino-3-mercapto]propylamino-3-(S)-methyl]pentyloxy-3-methybutanoyl-methionine,

Disulfide of 2(S)-[2(S)-[2(R)-amino-3-mercapto]propylamino-3(S)-methyl]pentyloxy-3-phenylpropionyl-homoserine lactone,

Disulfide of 2(S)-[2(S)-[2(R)-amino-3-mercapto]propylamino-3(S)-methyl]pentyloxy-3-phenylpropionyl-homoserine,

Disulfide of 2(S)-[2(S)-[2(R)-amino-3-mercapto]propylamino-3(S)-methyl]pentyloxy-3-methylbutanoyl-methionine methyl ester,

1-[2-(R)-amino-3-mercaptopropyl]-2(S)-(1-butyl)-4-(2,3-dimethylbenzoyl)piperazine dihydrochloride,

1-[2(R)-amino-3-mercaptopropyl]-2(S)-(n-butyl)-4-(1-naphthoyl)piperazine,

1-[2(R)-amino-3-mercaptopropyl]-2(S)-benzyl-4-[1-(2,3-dimethyl)benzoyl]piperazine,

1-[2(R)-amino-3-mercaptopropyl]-2(S)-(2-methoxy)ethyl-4-[ 1-(2,3-dimethyl)benzoyl]piperazine.,

1-[2(R)-amino-3-mercaptopropyl]-2(S)-(2-methylthio)ethyl-4-[1-(2,3-dimethyl)benzoyl]piperazine,

1-[2(R)-amino-3-mercaptopropyl]-2(S)-(n-butyl)-4-[7-(2,3-dihydrobenzofuroyl)]piperazine,

1-(2(R)-amino-3-mercaptopropyl)-4-(1-naphthoyl)-2(S)-pyridinylcarboxyl-4-piperazine dihydrochloride,

Methyl 4-(2(R)-amino-3-mercaptopropyl)-1-(1-naphthylmethyl)piperazine-2-carboxylate hydrochloride,

1-[2(R)-amino-3-mercaptopropyl]-2(S)-(2-methoxyethyl)-4-(1-naphthoyl)piperazine,

1-[2(R)-amino-3-mercaptopropyl]-2(S)-n-butyl-4-(8-quinolinylcarbonyl)piperazine,

1-[2(R)-amino-3-mercaptopropyl]-2(S)-(2-(1-propoxy)ethyl)-4-(1-naphthoyl)piperazine,

1-[2(R)-amino-3-mercaptopropyl)-2(S)-(3-methoxy-1-propyl)-4-(1-naphthoyl)piperazine,

1-[2(R)-amino-3-mercaptopropyl]-2(S)-(2-(1-propoxy)ethyl)-4-(8-quinolinoyl)piperazine,

1-[2(R)-amino-3-mercaptopropyl]-2(S)-[(3-pyridyl)methoxyethyl)]-4-(1-naphthoyl)piperazine,

1-[2(R)-amino-3-mercaptopropyl]-4-naphthoyl-2(S)-(2-phenylsulfonylethyl)piperazine dihydrochloride,

bis-1,1′-[2(R)-amino-3-(2(S)-(2-methoxyethyl)-4-naphthoyl-1-piperazinyl)]propyl disulfide tetrahydrochloride,

bis-1,1′-[2(R)-amino-3-(4-naphthoyl-2(S)-(2-phenylsulfonylethyl)-1-piperazinyl)]propyl disulfide tetrahydrochloride,

1-[2(R)-amino-3-mercaptopropyl]-4-naphthoyl-2(S)-(2-cyclopropyloxyethyl)piperazine dihydrochloride,

1-[2(R)-amino-3-mercaptopropyl]-4-(1-naphthoyl)-2(S)-(4-acetamidobutyl)piperazine dihydrochloride,

1-[2(R)-amino-3-mercaptopropyl]-4-naphthoyl-2(S)-(2-cyclopropylmethylsulfonylethyl)piperazine dihydrochloride,

Pyroglutamyl-valyl-phenylalanyl-methionine,

Pyroglutamyl-valyl-phenylalanyl-methionine methyl ester,

Pyroglutamyl-valyl-isoleucyl-methionine,

Pyroglutamyl-valyl-isoleucyl-methionine methyl ester,

Nicotinoyl-isoleucyl-phenylalanyt-methionine,

Nicotinoyl-isoleucyl-phenylalanyl-methionine methyl ester,

N-[2(S)-(L-pyroglutamylamino)-3-methylbutyl]phenylalanyl-methionine,

N-[2(S)-(L-pyroglutamylamino)-3-methylbutyl]phenylalanyl-methioninemethyl ester,

N-[5(S)-(L-pyroglutamylamino)-6(S)-methyl-2(R)-butyl-3 ,4(E)octenoyl]-methionine,

N-[5(S)-(L-pyroglutamylamino)-6(S)-methyl-2(R)-butyl-3,4(E)octenoyl]-methionine methyl ester,

N-[5(S)-((Imidazol-4-yl)acetylamino)-6(S)-methyl-2(R)-butyl-3,4(E)octenoyl]-methionine,

N-[5(S)-((Imidazol-4-yl)acetylamino)-6(S)-methyl-2(R)-butyl-3,4(E)octenoyl]-methionine methyl ester,

N-[5(S)-((Imidazol-4-yl-carbonylamino)-6(S)-methyl-2(R)-butyl-3,4(E)octenoyl]-methionine,

N-[5(S)-((Imidazol-4-yl-carbonylamino)-6(S)-methyl-2(R)-butyl-3,4(E)octenoyl]-methionine methyl ester,

N-[2(S)-(2(S)-(Imidazol-4-yl)acetylamino)-3(S)-methylpentyloxy)-3-phenylpropionyl]-methionine,

N-[2(S)-(2(S)-(Imidazol-4-yl)acetylamino)-3(S)-methylpentyloxy)-3-phenylpropionyl]-methionine methyl ester,

N-[2(S)-(2(S)-Pyroglutamylamino-3(S)-methylpentyloxy)-3-phenylpropionyl]-methionine,

N-[2(S)-(2(S)-Pyroglutamylamino-3(S)-methylpentyloxy)-3-phenylpropionyl]-methionine methyl ester,

N-[2(S)-(2(S)-Imidazol-4-yl-carbonyl)amino)-3(S)-methylpentyloxy)-3-phenylpropionyl]-methionine,

N-[2(S)-(2(S)-Imidazol-4-yl-carbonyl)amino)-3(S)-methylpentyloxy)-3-phenylpropionyl]-methionine methyl ester,

N-[2(S)-(2(S)-((3-Picolinyl)amino)-3(S)-methylpentyloxy)-3-phenylpropionyl]-methionine,

N-[2(S)-(2(S)-((3-Picolinyl)amino)-3(S)-methylpentyloxy)-3-phenylpropionyl]-methionine methyl ester,

N-[2(S)-(2(S)-((Histidyl)amino)-3(S)-methylpentyloxy)-3-phenylpropionyl]-methionine,

N-[2(S)-(2(S)-((Histidyl)amino)-3 (S)-methylpentyloxy)-3-phenylpropionyl]-methionine methyl ester,

N-Benzyl-N-[2(S)-((Imidazol-4-yl-carbonyl)amino)-3(S)-methylpentyl]glycyl-methionine,

N-Benzyl-N-[2(S)-((Imidazol-4-yl-carbonyl)amino)-3(S)-methylpentyl]glycyl-methionine methyl ester,

N-Benzyl-N-[2(S)-((Imidazol-4-yl-acetyl)amino)-3(S)-methylpentyl]glycyl-methionine,

N-Benzyl-N-[2(S)-((Imidazol-4-yl-acetyl)amino)-3(S)-methylpentyl]glycyl-methionine methyl ester,

N-Benzyl-N-[2(S)-((Pyroglutamyl)amino)-3(S)-methylpentyl]-glycylmethionine,

N-Benzyl-N-[2(S)-((Pyroglutamyl)amino)-3(S)-methylpentyl]-glycylmethionine methyl ester,

N-(1-Naphthylmethyl)-N-[2(S)-((imidazol-4-yl-carbonyl)amino)-3(S)-methylpentyl]-glycyl-methionine,

N-(1-Naphthylmethyl)-N-[2(S)-((imidazol-4-yl-carbonyl)amino)-3(S)-methylpentyl]-glycyl-methionine methyl ester,

N-(1Naphthylmethyl)-N-[2(S)-((imidazol-4-yl-acetyl)amino)-3(S)-methylpentyl]-glycyl-methionine,

N-(1-Naphthylmethyl)-N-[2(S)-((imidazol-4-yl-acetyl)amino)-3(S)-methylpentyl]-glycyl-methionine methyl ester,

N-(1-Naphthylmethyl)-N-[2(S)-((pyroglutamyl)amino-3(S)-methylpentyl]-glycyl-methionine,

N-(1-Naphthylmethyl)-N-[2(S)-((pyroglutamyl)amino-3(S)-methylpentyl]-glycyl-methionine methyl ester,

N-[1-(Pyroglutamylamino)cyclopent-1-yl-methyl]—N-(1-naphthylmethyl)-glycyl-methionine methyl ester,

N-[1-(Pyroglutamylamino)-cyclopent-1-yl-methyl]—N-(1-naphthytmethyl)-glycyl-methionine,

N-(2(S)-L-Histidylamino-3(S)-methylpentyl)-N-(benzylmethyl)-glycylmethionine methyl ester,

N-(2(S)-L-Histidylamino-3(S)-methylpentyl)-N-(benzylmethyl)glycylmethionine,

N-(2(S)-L-Histidylamino-3(S)-methylpentyl)-N-(1-naphthylmethyl)glycylmethionine methyl ester,

N-(2(S)-L-Histidylamino-3(S)-methylpentyl)-N-(1-naphthylmethyl)glycyl-methionine,

2(S)-[2(S)-(L-Pyroglutamyl)amino-3(S)-methylpentyloxy]-3-methylbutanoyl-methionine methyl ester,

2(S)-[2(S)-(L-Pyroglutamyl)amino-3(S)-methylpentyloxy]-3-methylbutanoyl-methionine,

2(S)-[2(S)-(Imidazol-4-yl-acetyl)amino-3(S)-methylpentyloxy]-3-methylbutanoyl-methionine methyl ester,

2(S)-[2(S)-(Imidazol-4-yl-acetyl)amino-3(S)-methylpentyloxy]-3-methylbutanoyl-methionine,

N-(Benzyl)-N-[2(S)-(2-oxopyrrolidin-5(R,S)-yl-methyl)amino-3(S)methylpentyl]-glycyl-methionine methyl ester,

N-(Benzvl)-N-[2(S)-(2-oxopyrrolidin-5(R,S)-yl-methyl)amino-3(S)methylpentyl]-glycyl-methionine,

N-(Benzyl)-N-(2(S)-[((D,L)-2-thiazolyl)alanyl)amino]-3(S)methylpentyl)-glycyl-methionine methyl ester,

N-(Benzyl)-N-(2(S)-[((D,L)-2-thiazolyl)alanyl)amino]-3(S)methylpentyl)-glycyl-methionine,

N-(Benzyl)-N-[2(S)-(3-pyridylmethyl)amino-3(S)-methylpentyl]-glycylmethionine methyl ester,

N-(Benzyl)-N-[2(S)-(3-pyridylmethyl)amino-3(S)-methylpentyl]-glycylmethionine,

2(S)-[2(S)-(2-Oxopyrrolidin-5(S)-yl-methyl)amino-3(S)methylpentyloxy]-3-phenylpropionyl-methionine methyl ester,

2(S)-[2(S)-(2-Oxopyrrolidin-S(S)-yl-methyl)amino-3(S)-methyl-pentyloxy]-3-phenylpropionyl-methionine,

2(S)-[2(S)-(L-Pyroglutamyl)amino-3(S)-methylpentyloxy]-3-(1-naphthyl)propionyl-methionine sulfone methyl ester,

2(S)-[2(S)-(L-Pyroglutamyl)amino-3(S)-methylpentyloxy]-3-(1-naphthyl)propionyl-methionine sulfone,

2(S)-[2(S)-(L-Pyroglutamyl)amino-3(S)-methylpentyloxy]-3-(2-naphthyl)propionyl-methionine sulfone methyl ester,

2(S)-[2(S)-(L-Pyroglutamyl)amino-3(S)-methylpentyloxy]-3-(2-naphthyl)propionyl-methionine sulfone,

2(S)-[2(S)-(Imidazol-4-yl-acetyl)amino-3(S)-methylpentyloxy]-3-(1-naphthyl)propionyl-methionine sulfone methyl ester,

2(S)-[2(S)-(Imidazol-4-yl-acetyl)amino-3(S)-methylpentyloxy]-3-(1-naphthyl)propionyl-methionine sulfone,

2(S)-[2(S)-(Imidazol-4-yl-acetyl)amino-3(S)-methylpentyloxy]-3-(2-naphthyl)propionyl-methionine sulfone methyl ester,

2(S)-[2(S)-(Imidazol-4-yl-acetyl)amino-3(S)-methylpentyloxy]-3-(2-naphthyl)propionyl-methionine sulfone,

N-[2(S)-(L-pyroglutamyl)amino-3(S)-methylpentyl]-N-(3-quinolylmethyl)glycyl-methionine methyl ester,

N-[2(S)-(L-pyroglutamyl)amino-3(S)-methylpentyl]-N-(3-quinolylmethyl)glycyl-methionine,

N-(Benzyl)-N-[2(S)-(tetrazol-1-yl-acetyl)amino-3(S)-methylpentyl]glycyl-methionine methyl ester,

N-(Benzyl)-N-[2(S)-(tetrazol-1-yl-acetyl)amino-3(S)-methylpentyl]glycyl-methionine,

N-(Benzyl)-N-[2(S)-nicotinoylamino-3(S)-methylpentyl]-glycylmethionine-methyl ester,

N-(Benzyl)-N-[2(S)-nicotinoylamino-3(S)-methylpentyl]-glycylmethionine,

N-[2(S)-(L-Pyroglutamyl)amino-3(S)-methylpentyl]-N-(1-naphthylmethyl)-glycyl-methionine sulfoxide methyl ester,

N-[2(S)-(L-Pyroglutamyl)amino-3(S)-methylpentyl]-N-(1-naphthylmethyl)-glycyl-methionine sulfoxide,

2(S)-(2(S)-[2(S,R)-(Imidazol-4-yl)-2-aminoacetyl)amino]-3(S)-methylpentyloxy)-3-phenylpropionyl-methionine sulfone methyl ester,

2(S)-(2(S)-[2(S,R)-(Imidazol-4-yl)-2-aminoacetyl)amino]-3(S)-methylpentyloxy)-3-phenylpropionyl-methionine sulfone,

2(S)-(2(S)-[2(R,S)-(Imidazol-4-yl)-2-aminoacetyl)amino]-3(S)-methylpentyloxy)-3-phenylpropionyl-methionine sulfone methylester,

2(S)-(2(S)-[2(R,S)-(Imidazol-4-yl)-2-aminoacetyl)amino]-3(S)-methylpentyloxy)-3-phenylpropionyl-methionine sulfone,

N-(2(S)-[2(S,R)-(Imidazol-4-yl)-2-aminoacetyl]amino-3(S)-methylpentyl)-N-(1-naphthylmethyl)-glycyl-methioninemethyl ester,

N-(2(S)-[2(S,R)-(Imidazol-4-yl)-2-aminoacetyl]amino-3(S)-methylpentyl)-N-(1-naphthylmethyl)-glycyl-methionine,

N-(2(S)-[2(R,S)-(Imidazol-4-yl)-2-aminoacetyl]amino-3(S)-methylpentyl)-N-(1-naphthylmethyl)-glycyl-methioninemethyl ester,

N-(2(S)-[2(R,S)-(Imidazol-4-yl)-2-aminoacetyl]amino-3(S)-methylpentyl)-N-(1-naphthylmethyl)-glycyl-methionine,

N-(2(S)-[(Imidazol-4-yl)methyl]amino-3(S)-methylpentyl)-N-(1-naphthylmethyl)-glycyl-methionine methyl ester,

N-(2(S)-[(Imidazol-4-yl)methyl]amino-3(S)-methylpentyl)-N-(1-naphthylmethyl)-glycyl-methionine,

N-[2(S)-(L-pyroglutamyl)amino-3(S)-methylpentyl]-N-(1-naphthylmethyl)glycyl-methionine isopropyl ester,

N-[2(S)-(L-pyroglutamyl)amino-3(S)-methylpentyl]-N-(1-naphthylmethyl)glycyl-methionine t-butyl ester,

N-[2(S)-(L-pyroglutamyl)amino-3(S)-methylpentyl]-N-(4-quinolyl-5-methyl)glycyl-methionine methyl ester,

N-[2(S)-(L-pyroglutamyl)amino-3 (S)-methylpentyl]-N-(4-quinolylmethyl)glycyl-methionine,

N-(2(S)-[3-(Imidazol-4-yl)propyl]amino-3(S)-methylpentyl)-N-(1-naphthylmethyl)glycyl-methionine methyl ester,

N-(2(S)-[3-(Imidazol-4-yl)propyl]amino-3(S)-methylpentyl)-N-(1-naphthylmethyl)glycyl-methionine,

N-[2(S)-(L-pyroglutamyl)amino-3(S)-methylpentyl]-N-(1-naphthylmethyl)glycyl-norleucine,

N-[2(S)-(L-pyroglutamyl)amino-3(S)-methylpentyl]-N-(1-naphthylmethyl)glycyl-norleucine methyl ester,

N-[2(S)-(L-pyroglutamyl)amino-3(S)-methylpentyl]-N-(1-naphthylmethyl)glycyl-glutarnine,

N-[2(S)-(L-pyroglutamyl)amino-3(S)-methylpentyl]-N-(1-naphthylmethyl)glycyl-glutamine t-butyl ester,

N-[2(S)-(L-pyroglutamyl)amino-3(S)-methylpentyl]-N-[5-(dimethylamino)naphthylsulfonyl]glycyl-methionine methyl ester,

N-[2(S)-(3-pyridylmethyl)amino-3(S)-methylpentyl]-N-(1-naphthylmethyl)glycyl-methionine,

2(S)-(2(S)-[2-(Imidazol-4-yl)ethyl]amino-3(S)-methylpentyloxy)-3-phenylpropionyl-methionine sulfone methyl ester,

2(S)-(2(S)-[2-(Imidazol-4-yl)ethyl]amino-3(S)-methylpentyloxy)-3-phenylpropionyl-methionine sulfone,

N-[2(S)-(L-pyroglutamyl)amino-3(S)-methylpentyl]-N-(1-naphthylmethyl)glycyl-serine methyl ester,

N-[2(S)-(L-pyroglutamyl)amino-3(S)-methylpentyl]-N-(1-naphthylmethyl)glycyl-(D,L)-serine,

N-[2(S)-(L-pyroglutamyl)amino-3(S)-methylpentyl]-N-(1-naphthylmethyl)glycyl-(L,D)-serine,

N-[2(S)-(L-pyroglutamyl)amino-3(S)-methylpentyl]-N-(1-naphthylmethyl)glycyl-homoserine lactone,

N-[2(S)-(L-pyroglutamyl)amino-3(S)-methylpentyl]-N-(1-naphthylmethyl)glycyl-homoserine,

N-[2(S)-(L-pyroglutamyl)amino-3(S)-methylpentyl]-N-(cinnamyl)glycyl-methionine methyl ester,

N-[2(S)-(L-pyroglutamyl)amino-3 (S)-methylpentyl]-N-(cinnamyl)glycyl-methionine,

N-(2(S)-[2-(Imidazol-4-yl)ethyl]amino-3(S)-methylpentyl)-N-(1-naphthylmethyl)glycyl-methionine methyl ester,

N-(2(S)-[2-(Imidazol-4-yl)ethyl]amino-3(S)-methylpentyl)-N-(1-naphthylmethyl)glycyl-methionine,

N-[2(S)-(L-pyroglutamyl)amino-3 (S)-methylpentyl]-N-(1-naphthyl-methyl)glycyl-alanine methyl ester,

N-[2(S)-(L-pyroglutamyl)amino-3(S)-methylpentyl]-N-(1-naphthyl-5-methyl)glycyl-alanine,

N-[2(S)-(D-pyroglutamyl)amino-3(S)-methylpentyl]-N-(1-naphthylmethyl)glycyl-methionine methyl ester,

N-[2(S)-(D-pyroglutamyl)amino-3(S)-methylpentyl]-N-(1-naphthylmethyl)glycyl-methionine,

2(S)-[2(S)-(L-Pyroglutamyl)amino-3(S)-methylpentyloxy]-3-phenylpropionyl-methionine sulfone methyl ester,

2(S)-[2(S)-(L-Pyroglutamyl)amino-3(S)-methylpentyloxy]-3-phenylpropionyl-methionine sulfone,

N-[2(S)-(L-pyroglutamyl)amino-3(S)-methylpentyl]-N-(2,3-methylenedioxybenzyl)glycyl-methionine methyl ester,

N-[2(S)-(L-pyroglutamyl)amino-3(S)-methylpentyl]-N-(2,3-methylenedioxybenzyl)glycyl-methionine,

N-[2(S)-(Imidazol-4-yl-acetyl)amino-3(S)-methylpentyl]-N-(2,3-dihydrobenzofuran-7-yl-methyl)glycyl-methionine methyl ester,

N-[2(S)-(Imidazol-4-yl-acetyl)amino-3(S)-methylpentyl]-N-(2,3-dihydrobenzofuran-7-yl-methyl)glycyl-methionine,

N-(2(S)-[3-(3-indolyl)propionyl]amino-3(S)-methylpentyl)-N-(1-naphthylmethyl)glycyl-methionine methyl ester,

N-(2(S)-[3-(3-indolyl)propionyl]amino-3(S)-methylpentyl)-N-(1-naphthylmethyl)glycyl-methionine,

N-(2(S)-[3-(1-indolyl)propionyl]aamino-3(S)-methylpentyl)-N-(1-naphthylmethyl)glycyl-methionine methyl ester,

N-(2(S)-[3-(1-indolyl)propionyl]amino-3(S)-methylpentyl)-N-(1-naphthylmethyl)glycyl-methionine,

N-[2(S)-(L-pyroglutamyl)amino-3(S)-methylpentyl]-N-(1-naphthylmethyl)glycyl-histidine methyl ester,

N-[2(S)-(L-pyroglutamyl)amino-3(S)-methylpentyl]-N-(1-naphthylmethyl)glycyl-histidine,

N-[2(S)-(L-pyroglutamyl)amino-3(S)-methylpentyl]-N-(cyclopropylmethyl)glycyl-methionine methylester,

N-[2(S)-(L-pyroglutamyl)amino-3 (S)-methylpentyl]-N-(cyclopropylmethyl)glycyl-methionine,

N-[2(S)-(Imidazol-4-yl-acetyl)amino-3(S)-methylpentyl]-N-(cyclopropylmethyl)glycyl-methionine methylester,

N-[2(S)-(Imidazol-4-yl-acetyl)amino-3(S)-methylpentyl]-N-(cyclopropylmethyl)glycyl-methionine,

N-[2(S)-(L-pyroglutarnyl)amino-3(S)-methylpentyl]-N-(2,3-dihydrobenzofuran-7-yl-methyl)glycyl-methionine methyl ester,

N-[2(S)-(L-pyroglutamyl)amino-3(S)-methylpentyl]-N-(2,3-dihydrobenzofuran-7-yl-methyl)glycyl-methionine,

2(S)-[2(S)-N-(L-Pyroglutamyl)-N-methylamino-3(S)-methylpentyloxy]-3-phenylpropionyl-methionine methyl ester,

2(S)-[2(S)-N-(L-Pyroglutamyl)-N-methylamino-3(S)-methylpentyloxy]-3-phenylpropionyl-methionine,

N-[2(S)-(L-pyroglutamyl)amino-3 (S)-N-ethylpentyl]-N-(1-naphthylmethyl)glycyl-O-methylserine methyl ester,

N-[2(S)-(L-pyroglutamyl)amino-3(S)-methylpentyl]-N-(1-naphthylmethyl)glycyl-O-methylserine,

N-(1-Naphthylmethyl)-N-[2(S)-(N′-(L-pyroglutamyl)-N′-methylaino)-3(S)-methylpentyl]-glycyl-methionine methyl ester,

N-(1-Naphthylmethyl)-N-[2(S)-(N′-(L-pyroglutamyl)-N′-methylamino)-3(S)-methylpentyl]-glycyl-methionine,

N-[1-(Pyroglutamylamino)cyclopent-1-yl-methyl]-N-(1-naphthylmethyl)glycyl-methionine methyl ester,

N-[1-(Pyroglutamylamino)-cyclopent-1-yl-methyl]-N-(1-naphthylmethyl)glycyl-methionine,

N-[2(S)-(Pyridin-2-on-6-yl-carbonyl)amino-3(S)-methylpentyl]-N-(1-naphthylmethyl)glycyl-methionine methyl ester,

N-[2(S)-(Pyridin-2-on-6-yl-carbonyl)amino-3(S)-methylpentyl]-N-(1-naphthylmethyl)glycyl-methionine,

N-[2(S)-(L-pyroglutamyl)amino-3(S)-methylpentyl]-N-(3-chlorobenzyl)glycyl-methionine methyl ester,

N-[2(S)-(L-pyroglutamyl)amino-3(S)-methylpentyl]-N-(3-chlorobenzyl)glycyl-methionine,

N-[2(S)-(L-pyroglutamyl)amino-3(S)-methylpentyl]-N-(1-naphthylmethyl)glycyl-O-methylhomoserine methyl ester,

N-[2(S)-(L-pyroglutamyl)amino-3 (S)-methylpentyl]-N-(1-naphthylmethyl)glycyl-O-methylhomoserine,

N-[2(S)-(L-pyroglutamyl)amino-3(S)-methylpentyl]-N-(2,3-dimethylbenzyl)glycyl-methionine methyl ester,

N-[2(S)-(L-pyroglutamyl)amino-3(S)-methylpentyl]-N-(2,3-dimethylbenzyl)glycyl-methionine,

N-[2(S)-(L-pyroglutamyl)amino-3(S)-methylpentyl]-N-(1-naphthylmethyl)glycyl-(2-thienyl)alanine methyl ester,

N-[2(S)-(L-pyroglutamyl)amino-3(S)-methylpentyl]-N-(1-naphthylmethyl)glycyl-(2-thienyl)alanine,

N-[2(S)-(pyrrolidin-2-on-1-yl)-3-methylbutanoyl]-isoleucyl-methionine,

N-[2(S)-(piperidin-2-on-1-yl)-3-methylbutanoyl]-isoleucyl-methionine,

or the pharmaceutically acceptable salts or optical isomers thereof.

(aa) compounds of the following formulae, which compounds are also disclosed in U.S. Pat. No. 5,260,465, incorporated herein by reference,

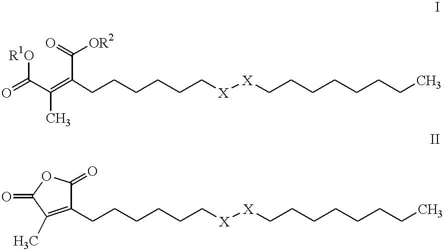

wherein:

X═X is:

CH═CH (cis);

CH═CH (trans); or

CH₂CH₂;

R¹ and R² are each independently selected from:

a) H;

b) C₁₋₅ alkyl;

C) C₁₋₅ alkyl substituted with a member of the group consisting of:

i) phenyl;

ii) phenyl substituted with methyl, methoxy, halogen (Cl, Br, F, I) or hydroxy;

or a pharmaceutically acceptable salt of a compound of formula (I) in which at least one of R¹ and R² is hydrogen;

(bb) compounds of the following formula, which compounds are also disclosed in U.S. Pat. No. 5,420,157, incorporated herein by reference,

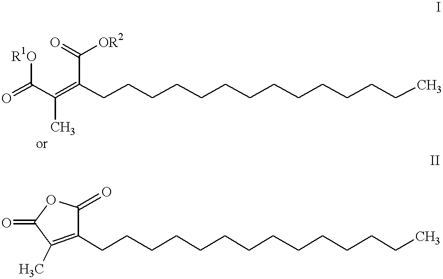

wherein:

R¹ and R² are each independently selected from:

a) H;

b) C₁₋₅ is alkyl;

c) C₁₋₅ is alkyl substituted with a member of the group consisting of:

i) phenyl;

ii) phenyl substituted with methyl, methoxy, halogen (Cl, Br, F, I) or hydroxy;

or a pharmaceutically acceptable salt of a compound of formula (I) in which at least one of R¹ and R² is hydrogen;

(cc) compounds of the following formulae, which compounds are also disclosed in U.S. Pat. Nos. 5,245,061 and 5,350,867, incorporated herein by reference,

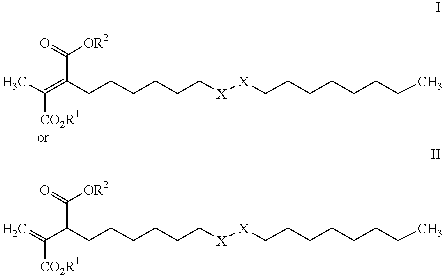

wherein:

X—X is:

CH═CH(cis);

CH═CH (trans); or

CH₂CH₂;

R¹ and R² are each independently selected from:

a) H;

b) C₁₋₃ alkyl;

c) C₁₋₅ alkyl substituted with a member of the group consisting of:

i) phenyl;

ii) phenyl substituted with methyl, methoxy, halogen (Cl, Br, F, I) or hydroxy;

or a pharmaceutically acceptable salt of a compound of formula (I) in which at least one of R¹ and R² is hydrogen;

(dd) compounds of the following formula, which compounds are also disclosed in PCT Publication No. WO 96/10037, incorporated herein by reference,

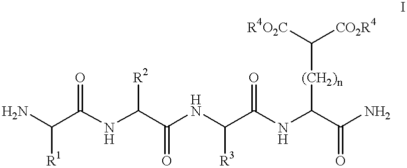

or the pharmaceutically acceptable salts, hydrates, esters or amides thereof, wherein:

n is 0 to 4,

R¹ and R³ independently are C₁₋₄ alkyl, substituted with substituents selected from the group consisting of:

a) aryl, which is defined as phenyl or naphthyl, unsubstituted or substituted with one, two, three or four substituents selected from the group consisting of:

i) F,

ii) Cl,

ii) Br,

iv) nitro,

v) cyano,

vi) C₁₋₈ alkoxy,

vii) C₁₋₈ alkylthio,

viii) C₁₋₈ alkylsulfonyl,

ix) sulfamoyl, or

x) C₁₋₈ alkyl; or

b) heteroaryl, which is defined as indolyl, imidazolyl or pyridyl, unsubstituted or substituted with one, two, three or four substituents selected from the group consisting of:

i) F,

ii) Cl,

iii) Br,

iv) nitro,

v) cyano,

vi) C₁₋₈ alkoxy,

vii) C₁₋₈ alkylthio,

viii) C₁₋₈ alkylsulfonyl,

ix) sulfamoyl, or

x) C₁₋₈ alkyl;

R² is: C₁₋₆ alkyl, which is unsubstituted or substituted with a substituent selected from the group consisting of:

a) unsubstituted or substituted aryl, as defined in R¹(a),

b) unsubstituted or substituted heteroaryl, as defined in R¹(b),

c) C₁₋₈ cycloalkyl,

d) C₁₋₈alkylthio,

e) C₁₋₈ alkylsulfonyl,

f) C₁₋₈ alkoxy, or

g) aryl C₁₋₈ alkyl sulfonyl; and

R⁴ is H;

(ee) compounds of the following formula, which are also disclosed in U.S. Pat. Nos. 5,298,655 and 5,362,906, incorporated herein by reference,

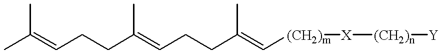

wherein:

X is CH,, CH(OH), C═O, CHCOR, CH(NH₂), CH(NHCOR), O, S(O)ₚ, NH, NHCO,

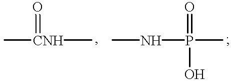

p is 0, 1 or 2;

Y is PO₃RR¹ or CO₂R;

R is H, lower alkyl, or CH₂CH₂N+Me₃A-;

R¹ is H, lower alkyl, or CH₂CH₂N+Me₃A-;

A is a pharmaceutically acceptable anion; m is 0, 1, 2, or 3; and n is 0, 1, 2, or 3;

(ff) compounds of the following formula, which compounds are also disclosed in PCT Publication No. WO 96/05169, incorporated herein by reference,

[chemical structure]

wherein each of

[chemical structure]

which are the same or different, is an aryl group or a heteroaromatic ring group; A is a C₂₋₈ saturated or unsaturated aliphatic hydrocarbon group which may have substituent(s) selected from the group consisting of a lower alkyl group, a hydroxyl group, a lower hydroxyalkyl group, a lower alkoxy group, a carboxyl group, a lower carboxyalkyl group, an aryl group and an aralkyl group; each of X and Y which are the same or different, is an oxygen atom, a sulfur atom, a carbonyl group or a group of the formula —CHR^{a}- (wherein Ra is a hydrogen atom or a lower alkyl group) or —NR^{b}(wherein Rb is a hydrogen atom or a lower alkyl group), or X and Y together represent

a vinylene group or an ethynylene group; each of R¹, R², R³, R⁸ and

R⁹ which are the same or different, is a hydrogen atom, a halogen atom, a hydroxyl group, a lower alkyl group or a lower alkoxy group;

each of R⁴ and R⁵ which are the same or different, is a hydrogen atom. a halogen atom, a hydroxyl group, an amino group, a nitro group, a cyano group, a carboxyl group, a lower alkoxycarbonyl group, a carbamoyl group, a lower alkylcarbamoyl group, a lower alkyl group, a lower hydroxyalkyl group, a lower fluoroalkyl group or a lower alkoxy group; R⁶ is a lower alkyl group; and R⁷ is a hydrogen atom or a lower alkyl group, provided that when one of X and Y is an oxygen atom, a sulfur atom or a group of the formula —NRb (wherein Rb is as defined above), the other is a carbonyl group or a group of the formula —CHR^{a}- (wherein Ra is as defined above);

(gg) compounds of the following formula, which compounds are also disclosed in PCT Publication No. WO 96/05168, incorporated herein by reference,

[chemical structure]

wherein each of

[chemical structure]

which are the same or different, is an aryl group or a heteroaromatic ring group; A is a C₂₋₈ saturated or unsaturated aliphatic hydrocarbon group which may have substituent(s) selected from the group consisting of a lower alkyl group, a hydroxyl group, a lower hydroxyalkyl group, a lower alkoxy group, a carboxyl group, a lower carboxyalkyl group, an aryl group and an aralkyl group; Q is a group of the formula —(CH₂)ₘ- (wherein m is an integer of from 1 to 6) or —(CH₂)ₙ—W(CH₂)p— (wherein W is an oxygen atom, a sulfur atom, a vinylene group or an ethynylene group; and each of n and p which are the same or different, is an integer of from 0 to 3); R¹ is a hydrogen atom, a halogen atom, a hydroxyl group, a lower alkyl group. a lower alkoxy group, or an aryl or heteroaromatic ring group which may have substituent(s) selected from the group consisting of a halogen atom, a lower alkyl group and a lower alkoxy group; each of R², R⁷ and R⁸ which are the same or different, is a hydrogen atom, a halogen atom, a hydroxyl group, a lower alkyl group or a lower alkoxy group; each of R³ and R⁴ which are the same or different, is a hydrogen atom, a halogen atom, a hydroxyl group, an amino group, a nitro group, a cyano group, a carboxyl group, a lower alkoxycarbonyl group, a carbamoyl group, a lower alkylcarbamoyl group, a lower alkyl group, a lower hydroxyalkyl group, a lower fluoroalkyl group or a lower alkoxy group; R⁵ is a lower alkyl group; and R⁶ is a hydrogen atom or a lower alkyl group;

or the pharmaceutically acceptable salts thereof.

Specifically suitable compounds of the above type include the following:

3-Hydroxy-7,11,15-trimethylhexadeca-6,10,14-trienoic acid,

[2-Oxo-6, 10,1 4-trimethylpentadec-5,9, 1 3-trienyl]phosphonic acid,

[2-Hydroxy-6,10,14-trimethylpentadec 5,9,13-trienyl]phosphonic acid,

[1-Acetyl-4,8,12-trimethylpentadeca-3,7,11-trienyl]phosphonic acid,

[2-[(E,E)-3 ,7,11-Trimethyl-2,6,10-dodecatrienylamino]-2-oxo-ethyl]phosphonic acid,

[(E,E)-4,8,12-Trimethyl-3,7,11-tridecatrienyl]thiomethyl-phosphonic acid,

3-[(E,E)-3 ,7,11-Trimethyl-2,6,10-dodecatrienylamino]-3-oxo-propionic acid,

[2-[(E,E)-3,7,11-Trimethyl-2,6,10-dodecatrienylamino]-2-oxoethyl]phosphonic acid monomethyl ester,

[2-[(E,E)-3,7,11-Trimethyl-2,6,10-dodecatrienylamino]-1-oxomethyl]phosphonic acid,

[1-Hydroxy-(E,E)-3,7,11-trimethyl-2,6,10-dodecatrienyl]-phosphonic acid,

[1-Hydroxy-(E,E)-5,9,13-trimethyl-4,8,12-tetradecatrienyl]-phosphonic acid,

[1-Hydroxy-(E,E)-4,8,12-trimethyl-3,7,11-tridecatrienyl]-phosphonic acid,

[2-Acetamido-(E,E)-4,8,12-trimethyl-3,7,11-tridecatrienyl]-phosphonic acid,

[2-Hydroxy-(E,E)-4,8,12-trimethyl-3,7,11-tridecatrienyl]-phosphonic acid,

N-((1RS,2RS,4E)-2-(4-chlorophenyl)-1-methyl-5-(2-naphthyl)-4-pentenyl)-N-(2-naphthylmethyl)-carbamoylmethyl succinic acid,

N-((1RS,2RS,4E)-2-(4-chlorophenyl)-1-methyl-5-(1-naphthyl)-4-pentenyl)-N-(2-naphthylmethyl)-carbamoylmethyl succinic acid,

N-((1RS,2RS)-2-(4-chlorophenyl)-1-methyl-5-(2-naphthyl)pentyl)-N-(2S naphthylmethyl)carbamoylmethyl succinic acid,

N-((1RS,2RS)-2-(4-chlorophenyl)-1-methyl-4-(2-naphthoxy)butyl)-N(2-naphthylmethyl)carbamoylmethyl succinic acid,

N-((1RS,2RS)-2-(4-chlorophenyl)-1-methyl-4-(2-naphthyl)butyl)-N-(2-naphthylmethyl)carbamoylmethyl succinic acid,

N-((1RS,2RS)-2-(4-chlorophenyl)-1-methyl-6-(2-naphthyl)hexyl)-N-(2-naphthylmethyl)carbamoylmethyl succinic acid,

N-((1RS,2RS)-2-(4-chlorophenyl)-1-methyl-5-phenyl-4-pentynyl)-N-(2-naphthylmethyl)carbamoylmethyl succinic acid,

N-((1RS ,2RS ,4E)-2-(4-methoxyphenyl)-1-methyl-5-(2-naphthyl)-4-pentenyl)-N-(2-naphthylmethyl)carbamoylmethyl succinic acid,

N-((1RS,2RS,4E)-1-methyl-2-(4-methylphenyl)-5-(2-naphthyl)-4-pentenyl)-N-(2-naphthyl-methyl)carbamoylmethyl succinic acid,

N-((1RS,2RS,4E)-1-methyl-5-(2-naphthyl)-2-(4-nitrophenyl)-4-pentenyl)-N-(2-naphthylmethyl)carbamoylmethyl succinic acid,

N-((1RS,2RS,4E)-2-(4-fluorophenyl)-1-methyl-5-(2-naphthyl)-4-pentenyl)-N-(2-naphthylmethyl)carbamoylmethyl succinic acid,

N-((1RS,2RS,4E)-1-methyl-5-(2-naphthyl)-2-(4-trifluoromethylphenyl)4-pentenyl)-N-(2-naphthylmethyl)carbamoylmethyl succinic acid,

N-((1RS,2RS,4E)-1-methyl-5-(2-naphthyl)-2-phenyl-4-pentenyl)-N-(2-naphthylmethyl)carbamoylmethyl succinic acid,

N-((1RS,2RS,4E)-1-methyl-2-(6-methyl-3-pyridyl)-5-(2-naphthyl)-4-pentenyl)-N-(2-naphthylmethyl)carbamoylmethyl succinic acid,

N-((1RS,2RS,6E)-2-(4-chlorophenyl)-1-methyl-7-phenyl-6-heptenyl)-N-(2-naphthylmethyl)carbamoylmethyl succinic acid,

N-((1RS,2RS,6E)-2-(4-chlorophenyl)-1-methyl-7-(2-naphthyl)-6-heptenyl)-N-(2-naphthylmethyl)carbamoylmethyl succinic acid,

N-((1RS,2RS,4E)-2-(4-chlorophenyl)-1-methyl-5-(2-naphthyl)-4-pentenyl)-N-(3-quinolylmethyl)carbarnoylmethyl succinic acid,

N-((1RS,2RS,4E)-2-(4-chlorophenyl)-1-methyl-5-(2-naphthyl)-4-pentenyl)-N-(3,4-difluorobenzyl)carbamoylmethyl succinic acid,

N-(2-benzoxazolylmethyl)-N-((1RS,2RS,4E)-2-(4-chlorophenyl)-1-methyl-5-(2-naphthyl)-4-pentenyl)carbamoylmethyl succinic acid,

N-(2-benzo[b]thienylmethyl)-N-((1RS,2RS,4E)-2-(4-chlorophenyl)-1-methyl-5-(2-naphthyl)-4-pentenyl)carbamoylmethyl succinic acid,

N-((1RS,2RS,4E)-1-methyl-2-(3 ,4-methylenedioxyphenyl)-5-(2-naphthyl)-4-pentenyl)-N-(2-naphthylmethyl)carbamoylmethyl succinic acid,

(2R*)-2-[N-((1 S*,2S*,4E)-2-(4-chlorophenyl)-1-methyl-5-(2-naphthyl)-4-pentenyl)-N-(2-naphthylmethyl)carbarmoylmethyl]succinic acid,

(2R*)-2-[N-((1R*,2R*,4E)-2-(4-chlorophenyl)-1-methyl-5-(2-naphthyl)-4-pentenyl)-N-(2-naphthylmethyl)carbamoylmethyl]succinic acid,

(2S *)-2-[N-((1R*,2R*,4E)-2-(4-chlorophenyl)--methyl-5-(2-naphthyl)-4-pentenyl)-N-(2-naphthylmethyl)carbarnoylmethyl]succinic acid,

(2S *)-2-[N-(( iS*,2S*,4E)-2-(4-chlorophenyl)-1-methyl-5-(2-naphthyl)-4-pentenyl)-N-(2-naphthylmethyl)carbamoylmethyl]succinic acid,

5-[N-((1RS,2RS,4E)-2-(4-chlorophenyl)-1-methyl-5-(2-naphthyl)-4-pentenyl)-N-(2-naphthylmethyl)carbamoyl]pentanoic acid,

(2R*)-2-[N-((1RS,2RS,4E)-5-(2-benzoxazolyl)-1-methyl-2-(3,4-methylenedioxyphenyl)-4-pentenyl)-N-(2-naphthylmethyl)carbamoylrethyl]succinic acid,

(2R*)-2-[N-((1RS,2RS,4Z)-5-(2-benzoxazolyl)-1-methyl-2-(3,4-methylenedioxyphenyl)-4-pentenyl)-N-(2-naphthylmethyl)carbamoylmethyl]succinic acid,

(2R*)-2-[N-(2-benzo[b]furanylmethyl)-N-((1RS.2RS,4E)-5-(2-benzoxazolyl)-11-methyl-2-(3,4-methylenedioxyphenyl)-4-pentenyl)carbamoylmethyl]succinate,

(2R*)-2-[N-(2-benzo[b]thienylmethyl)-N-((RS,2RS,4E)-5-(2-benzoxazolyl)-1-methyl-2-(3,4-methylenedioxyphenyl)-4-pentenyl)carbamoylmethyl]succinic acid,

(2R*)-2-[N-[(1RS,2RS,4E)-5-(2-benzoxazolyl)-2-(3,4-bis(methoxycarbonyl)phenyl)-1-methyl-4-pentenyl]-N-(2-naphthylmethyl)carbamoylmethyl]succinic acid,

(2R*)-2-[N-(2-benzo[b]thienylmethyl)-N-((1RS,2RS,4E)-5-(2-benzoxazolyl)-2-(4-methoxycarbonylphenyl)-1-methyl-4-pentenyl)carbamoylmethyl]succinic acid,

(2R*)-2-[N-(2-benzo [b]fuiranylmethyl)-N-((1RS,2RS,4E)-5-(2-benzoxazolyl)-25 2-(4-methoxycarbonylphenyl)-1-methyl-4-pentenyl)carbamoylmethyl]succinic acid,

(2R*)-2-[N-(2-benzo[b]thienylmethyl)-N-((1RS,2RS,4E)-5-(2-benzoxazolyl)-2-(4-cyanophenyl)-1-methyl-4-pentenyl)carbamoylmethyl]succinic acid,

(2R*)-2-[N-(5-benzo[b]thienylmethyl)-N-((1RS,2RS,4E)-5-(2-benzoxazolyl)-2-(4-methoxycarbonylphenyl)-1-methyl-4-pentenyl)carbamoylmethyl]succinic acid,

30 N-((1RS,2RS,4E)-5-(3-chloro-4-methylphenyl)-2-(4-chlorophenyl)-1-methyl-4-pentenyl)-N-(2-naphthylmethyl)carbamoylmethylsuccinic acid,

N-((1RS,2RS,4Z)-5-(3-chloro-4-methylphenyl)-2-(4-chlorophenyl)-1-methyl-4-pentenyl)-N-(2-naphthylmethyl)carbamoylmethylsuccinic acid,

N-((1RS,2RS,4E)-5-(2-benzo[b]furanyl)-2-(4-chlorophenyl)-1-methyl-4-pentenyl)-N-(2-naphthylmethyl)carbamoylmethylsuccinic acid,

N-((1RS,2RS,4Z)-5-(2-benzo[b]furanyl)-2-(4-chlorophenyl)-1-methyl-4-pentenyl)-N-(2-naphthylmethyl)carbamoylmethylsuccinic acid,

N-((1RS,2RS,4E)-5-(2-benzoxazolyl)-2-(4-chlorophenyl)-1-methyl-4-pentenyl)-N-(2-naphthylmethyl)carbamoylmethylsuccinic acid,

N-((1RS,2RS,4Z)-5-(2-benzoxazolyl)-2-(4-chlorophenyl)-1-methyl-4-pentenyl)-N-(2-naphthylmethyl)carbamoylmethyl succinic acid,

N-((1RS,2RS,4E)-5-(2-benzimidazolyl)-2-(4-chlorophenyl)-1-methyl-4-pentenyl)-N-(2-naphthylmethyl)carbamoylmethylsuccinic acid,

N-((1RS,2RS,4E)-2-(4-chlorophenyl)-1-methyl-5-(3,4-methylenedioxyphenyl)-4-pentenyl)-N-(2-naphthylmethyl)carbamoylmethylsuccinic acid,

N-((1RS,2RS ,4E)-5-(2-benzothiazolyl)-2-(4-chlorophenyl)-1-methyl-4-pentenyl)-N-(2-naphthylmethyl)carbamoylmethylsuccinic acid,

N-((1RS,2RS,4E)-5-(2-benzoxazolyl)-2-(4-cyanophenyl)-1-methyl-4-pentenyl)-N-(2-naphthylmethyl)carbamoylmethylsuccinic acid,

4-[N-((1RS,2RS,4E)-5-(2-benzoxazolyl)-1-methyl-2-(3,4-methylenedioxyphenyl)-4-pentenyl)-N-(2-naphthylmethyl)carbamoyl]-1,2,3-butanetricarboxylic acid,

3-[N-((1RS,2RS,4E)-5-(2-benzoxazolyl)-1-methyl-2-(3,4-methylenedioxyphenyl)-4-pentenyl)-N-(2-naphthylmethyl)carbamoyl]1,2,2-propanetricarboxylic acid,

(2S,3R)-4-[N-(( I RS,2RS,4E)-5-(2-benzoxazolyl)-1-methyl-2-(3,4-methylenedioxyphenyl)-4-pentenyl)-N-(2-naphthylmethyl)carbamoylmethyl]-3-carboxy-2-hydroxybutanoic acid,

4-[N-((1RS,2RS,4E)-5-(2-benzoxazolyl)-1-methyl-2-(3,4-methylenedioxyphenyl)-4-pentenyl)-N-(2-naphthylmethyl)carbamoyl]-3-carboxy-4-methioxybutanoic acid,

5-[N-((1RS,2RS,4E)-5-(2-benzoxazolyl)-1-methyl-2-(3,4-methylenedioxyphenyl)-4-pentenyl)-N-(2-naphthylmethyl)carbamoyl]-4-carboxy-3-carboxymethyl pentanoic acid,

1-[N-((1RS,2RS,4E)-5-(2-benzoxazolyl)-2-(4-methoxycarbonylphenyl)-1-methyl-4-pentenyl)-N-(2-naphthylmethyl)carbamoyl]-1,2,3-propanetricarboxylic acid,

(3R*)-4-[N-((1RS,2RS,4E)-5-(2-benzoxazolyl)-1-methyl-2-(3,4-methylenedioxyphenyl)-4-pentenyl)-N-(2-naphthylmethyl)carbarnoyl]-3-methoxybutanoic acid,

(3 S*)-4-[N-(( I RS,2RS,4E)-5-(2-benzoxazolyl)-1-methyl-2-(3,4-methylenedioxyphenyl)-4-pentenyl)-N-(2-naphthylmethyl)carbamoyl]-3-methoxybutanoic acid,

N-((1RS,2RS,4E)-5-(2-benzoxazolyl)-2-(4-carboxyphenyl)-1-methyl-4-pentenyl)-N-(2-naphthylmethyl)carbamoylmethylsuccinic acid,

N-((1RS,2RS,4E)-5-(2-benzoxazolyl)-1-methyl-2-(4- (N-methylcarbamoyl)phenyl)-4-pentenyl]-N-(2-naphthylmethyl)carbamoylmethylsuccinic acid,

(2R*)-2-[N-((1RS,2RS,4E)-5-(2-benzoxazolyl)-2-(4-hydroxy-3-methoxyphenyl)-1-methyl-4-pentenyl)-N-(2-naphthylmethyl)carbamoylmethylsuccinic acid,

N-((1RS,2RS,4E)-2-(4-hydroxymethylphenyl)-1-methyl-5-(2-naphthyl)-4-pentenyl)-N-(2-naphthylmethyl)carbamoylmethylsuccinic acid,

N-(1RS,2RS,4E)-2-(4-aminophenyl)-1-methyl-5-(2-naphthyl)-4-pentenyl)-N-(2-naphthylmethyl)carbamoylmethylsuccinic acid,

disodium (3RS,4RS)-4-[N-((1RS,2RS,4E)-5-(2-benzoxazolyl)-1-methyl-2-(3,4-methylenedioxyphenyl)-4-pentenyl)-N-(2-naphthylmethyl)carbamoyl]-3-carboxy-4-hyroxybutanoate,

N-((1RS,2RS,4E)-5-(2-benzoxazolyl)-1-methyl-2-(3,4-methylenedioxyphenyl)-4-pentenyl)-N-(2-naphthylmethyl)-5-oxotetrahydrofuran-2-carboxyamide,

sodium 4-[N-((1RS,2Rs,4E)-5-(2-benzoxazolyl)-1-methyl-2-(3,4-methylenedioxyphenyl)-4-pentenyl)-N-(2-naphthylmethyl)]carbamoyl-4-hyroxybutanoate,

4-[N-((1RS,2RS,4E)-5-(2-benzoxazolyl)-1-methyl-2-(3,4-methylenedioxyphenyl)-4-pentenyl)-N-(2-naphthylmethyl)carbamoyl]-2-oxotetrahydrofiran-3-yl-acetic acid,

(2R*)-2-[N-((1R*,2R*,⁴E)-5-(2-benzoxazolyl)-2-(4-methoxycarbonylphenyl)-1-methyl-4-pentenyl)-N-(2-naphthylmethyl)carbamoylmethyl]succinic acid,

(2R*)-2-[N-(( i S*,2S*,4E)-5-(2-benzoxazolyl)-2-(4-methoxycarbonylphenyl)-1-methyl-4-pentenyl)-N-(2-naphthylmethyl)carbamoylmethyl]succinic acid,

(2R*)-2-[N-(2-benzo[b]thienylmethyl)-N-(1 S*,2S*,4B)-5-(2-benzoxazolyl)-1-methyl-2-(3,4-methylenedioxyphenyl)-4-pentenyl)carbamoylmethyl]succinic acid,

(2R*)-2-[N-(2-benzo[b]thienylmethyl)-N-((1R*,2R*,4E)-5-(2-benzoxazolyl)-1-methyl-2-(3,4-methylenedioxyphenyl)-4-pentenyl)carbamoylmethyl]succinic acid,

(2R*)-2-[N-((1RS.2RS)-5-(2-benzoxazolyl)-l -methyl-2-(3,4-methylenedioxyphenyl)pentyl)-N-(2-naphthylmethyl)carbamoylmethyl]succinic acid,

(2R*)-2-[N-(2-benzo[b]thienylmethyl)-N-(( RS,2RS)-5-(2-benzoxazolyl)-1-methyl-2-(3,4-methylenedioxyphenyl)pentyl)carbamoylmethyl]succinic acid,

(2R*)-2-[N-((1R*,2R*)-5-(2-benzoxazolyl)-2-(4-methoxycarbonylphenyl)-1-methylpentyl)-N-(2-naphthylmethyl)carbamoylmethyl]succinic acid,

disodium (3 S,4S)-4-[N-((1R,2R,4E)-5-(2-benzoxazolyl)-methyl-2-(3,4-methylenedioxyphenyl)-4-pentenyl)-N-(2-naphthylmethyl)carbamoyl]-3-carboxy-4-hyroxybutanoate,

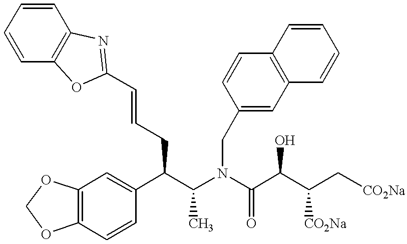

sodium (3S,45)-4-[N-((1R,2R,4E)-5-(2-benzoxazolyl)-1-methyl-2-(3,4-methylenedioxyphenyl)-4-pentenyl)-N-(2-naphthylmethyl)carbamoyl]-3-ethoxycarbonyl-4-hyroxybutanoate,

4-[N-((1R,2R,4E)-5-(2-benzoxazolyl)-1-methyl-2-(3,4-methylenedioxyphenyl)-4-pentenyl)-N-(2-naphthylmethyl)carbamoyl]-3-tert-butoxycarbonyl-4-hydroxy-3-butenoic acid,

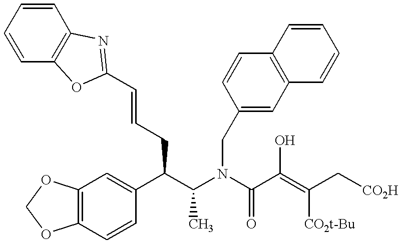

4-[N-((1R,2R,4E)-5-(2-benzoxazo lyl)-1-methyl-2-(3,4-methylenedioxyphenyl)-4-pentenyl)-N-(2-naphthylmethyl)carbarnoyl]-4-hydroxy-3-methoxycarbonyl-3-butenoic acid,

4-[N-((1R,2R,4E)-5-(2-benzoxazolyl)-1-methyl-2-(3,4-methylenedioxyphenyl)-4-pentenyl)-N-(2-naphthylmethyl)carbamoyl]-4-hydroxy-3-isopropoxycarbonyl-3-butenoic acid,

4-[N-((1R,2R,4E)-5-(2-benzoxazolyl)-1-methyl-2-(3,4-methylenedioxyphenyl)-4-pentenyl)-N-(2-naphthylmethyl)carbamoyl]-3-cyclohexyloxycarbonyl-4-hydroxy-3-butenoic acid,

4-[N-((1R,2R,4E)-5-(2-benzoxazolyl)-1-methyl-2-(3,4-methylenedioxyphenyl)-4-pentenyl)-N-(2-naphthylmethyl)carbamoyl]-4-hydroxy-3-(2-methoxyethoxy)carbonyl-3-butenoic acid,

4-[N-((1R,2R,4E)-5-(2-benzoxazolyl)-1-methyl-2-(3,4-methylenedioxyphenyl)-4-pentenyl)-N-(2-naphthylmethyl)carbamoyl]-3-benzyloxycarbonyl-4-hydroxy-3-butenoic acid,

4-[N-((1R,2R,4E)-5-(2-benzoxazolyl)-1-methyl-2-(3,4-methylenedioxyphenyl)-4-pentenyl)-N-(2-naphthylmethyl)carbamoyl]-3-cyclopentyloxycarbonyl-4-hydroxy-3-butenoic acid,

4-[N-((1R,2R,4E)-5-(2-benzoxazolyl)-1-methyl-2-(3,4-methylenedioxyphenyl)-4-pentenyl)-N-(2-naphthylmethyl)carbamoyl]-4-hydroxy-3-(3-tetrahydrofuranyloxycarbonyl)-3-butenoic acid,

4-[N-(( IR,2R,4E)-5-(2-benzoxazolyl)-1-methyl-2-(3,4-methylenedioxyphenyl)-4-pentenyl)-N-(2-naphthylmethyl)carbamoyl]-4-hydroxy-3-(2-hydroxy-1-hydroxymethylethoxycarbonyl)-3-butenoic acid,

3-allyloxycarbonyl-4-[N-((1R,2R,4E)-5-(2-benzoxazolyl)-1-methyl-2-(3,4-methylenedioxyphenyl)-4-pentenyl)-N-(2-naphthylmethyl)carbamoyl]-4-hydroxy-3-butenoic acid,

4-[N-((1R,2R,4E)-5-(2-benzoxazolyl)-2-(3,4-methylenedioxyphenyl)-1-methyl-4-pentenyl)-N-(2-naphthylmethyl)carbamoyl]-3-carboxymethylcarbonyl-4-hydroxy-3-butenoic acid,

5-[N-((1R,2R,4E)-5-(2-benzoxazolyl)-1-methyl-2-(3,4-methylenedioxyphenyl)-4-pentenyl)-N-(2-naphthylmethyl)carbamoyl]-4-ethoxycarbonyl-5-hydroxy-4-pentenoic acid,

5-N-((1R,2R,4E)-5-(2-benzoxazolyl)-1-methyl-2-(3,4-methylenedioxyphenyl)-4-pentenyl)-N-(2-naphthylmethyl)carbamoyl]-4-tert-butoxycarbonyl-5-hydroxy-4-pentenoic acid,

4-N-((1R,2R,4E)-5-(2-benzoxazolyl)-1-methyl-2-(3,4-methylenedioxyphenyl)-4-pentenyl)-N-(2-naphthylmethyl)carbamoyl]-4-hydroxy-3-hydroxymethyl-3-butenoic acid,

4-[N-((1RS,2RS,4E)-6-(2-benzoxazolyl)-1-methyl-2-(3,4-methylenedioxyphenyl)-5-hexenyl)-N-(2-naphthylmethyl)carbamoyl]-3-tert-butoxycarbonyl-4-hydroxy-3-butenoic acid,

(2S*,3R*)-4-[N-(( l R,2R,4E)-5-(2-benzoxazolyl)-1-methyl-2-(3,4-methylenedioxyphenyl)-4-pentenyl)-N-(2-naphthylmethyl)carbamoyl]-1,2,3-butanetricarboxylic acid,

(2R*,3S*)-4-[N-((1R.2R,4E)-5-(2-benzoxazolyl)-1-methyl-2-(3,4-methylenedioxyphenyl)-4-pentenyl)-N-(2-naphthylmethyl)carbamoyl]-1,2,3-butanetricarboxylic acid,

N-[(1RS,2RS)-1-methyl-2-(4-nitrophenyl)-3-(5- (phenylcarbamoyl)-2-furyl)-propyl]-N-(2-naphthylmethyl)carbamoylmethylsuccinic acid,

N-[(1RS,2RS)-3-(5-(3,4-dimethoxyphenylcarbamoyl)-2-furyl)-1-methyl-2-(4-nitrophenyl)propyl]-N-(2-naphthylmethyl)carbamoylmethylsuccinic acid,

N-[(1RS,2RS)-3-(5-(2-hydroxyphenylcarbamoyl)-2-furyl)-1-methyl-2-(4-nitrophenyl)propyl]-N-(2-naphthylmethyl)carbamoylmethylsuccinic acid,

N-[(1RS,2RS)-1-methyl-3-(5- (N-methylphenylcarbamoyl)-2-furyl)-2-(4-nitrophenyl)propyl]-N-(2-naphthylmethyl)carbamoylmethylsuccinic acid,

N-[(1RS,2RS)-1-methyl-2-(4-nitrophenyl)-3-(5-(3-pyridylcarbamoyl)-2-furyl)-propyl]-N-(2-naphthylmethyl)carbamoylmethylsuccinic acid,

N-[(1RS,2RS)-1-methyl-2-(4-nitrophenyl)-3-(5-(4-pyridylcarbamoyl)-2-furyl)-propyl]-N-(2-naphthylmethyl)carbamoylmethylsuccinic acid,

N-[(1RS,2RS)-1-methyl-2-(4-nitrophenyl)-3-(5- (spyrimidinylcarbamoyl)-2-furyl)-propyl]-N-(2-naphthylmethyl)carbamoylmethylsuccinic acid,

N-[(1RS,2RS)-1-methyl-2-(4-nitrophenyl)-3-(5-(2-thiazolylcarbamoyl)-furyl)-propyl]-N-(2-naphthylmethyl)carbamoylmethylsuccinic acid

N-[(1RS,2RS)-2-(4-chlorophenyl)-1-methyl-3-(5- (phenylcarbamoyl)-2-furyl)-propyl]-N-(2-naphthylmethyl)carbarnoylmethylsuccinic acid,

N-((1RS,2RS)-2-(4-chlorophenyl)-1-methyl-3-(3-phenylcarbamoylphenyl)propyl)-N-(2-naphthylmethyl)carbamoylmethylsuccinic acid,

N-[(1RS,2RS)-2-(4-chlorophenyl)-1-methyl-3-(3- (phenylcarbamoyl)-5-isoxazolyl)-propyl]-N-(2-naphthylmethyl)carbamoylmethylsuccinic acid,

N-[(1RS,2RS)-2-(4-chlorophenyl)-1-methyl-3-(4- (phenylcarbamoyl)-2-pyridyl)-propyl]-N-(2-naphthylmethyl)carbamoylmethylsuccinic acid,

(2R*)-2-[N-(2-benzo[b]thienylmethyl)-N-[(1RS,2RS)-1-methyl-2-(3,4-methylenedioxyphenyl)-3-(5- (phenylcarbamoyl)-2-furyl)-propyl]carbamoylmethyl]succinic acid,

(2R*)-2-[N-(2-benzo[b]thienylmethyl)-N-[(1RS,2RS)-1-methyl-2-(3,4-methylenedioxyphenyl)-3-(5-(3-pyridylcarbamoyl)-2-furyl)propyl]carbamoylmethyl]succinic acid,

monopivaloyloxymethyl (2R*)-2-[N-(2-benzo[b)thienylmethyl)-N[(1RS,2RS)-1-methyl-2-(3,4-methylenedioxyphenyl)-3-(5-(phenylcarbamoyl)-2-furyl)propyl]carbamoylmethyl]succinate

(2R*)-2-[N-((1RS,2RS)-2-(4-methoxycarbonylphenyl)-1-methyl-3-(3-phenoxymethylphenyl)propyl)-N-2-naphthylmethyl)carbamoylmethyl]succinic acid,

(2R*)-2-[N-[(1RS,2RS)-2-(4-methoxycarbonylphenyl)-1-methyl-3-(3-(phenoxymethyl)-5-(1,2,4-oxadiazolyl))propyl]-N-(2-naphthylmethyl)carbamoylmethyl]succinic acid,

(2R*)-2-[N-[(IRS,2RS)-2-(4-methoxycarbonylphenyl)-1-methyl-3- ((E)-3-styrylphenyl)propyl]-N-(2-naphthylmethyl)carbamoylmethyl]succinic acid,

(2R*)-2-[N-[(1RS,2RS)-2-(4-methoxycarbonylphenyl)-1-methyl-3-(3-(2-phenylethyl)phenyl)propyl]-N-(2-naphthylmethyl)carbamoylmethyl]succinic acid,

N-((1RS,2RS)-2-(4-chlorophenyl)-1-methyl-3-(4-phenylethynylphenyl)propyl)-N-(2-naphthylmethyl)carbamoylmethylsuccinic acid,

N-[(1RS,2RS)-2-(4-chlorophenyl)-1-methyl-3- ((E)-3-styrylphenyl)propyl]-N-(2-naphthylmethyl)carbamoylmethylsuccinic acid

N-((1RS,2RS)-2-(4-methoxycarbonylphenyl)-1-methyl-3-(5-phenoxymethyl-2-fiuryl)propyl)-N-(2-naphthylmethyl)carbamoylmethylsuccinic acid,

4-[N-[(1RS,2RS)-1-methyl-2-(4-nitrophenyl)-3-(5- (phenylcarbamoyl)-2-furyl)propyl]-N-(2-naphthylmethyl)carbamoyl]-1,2,3-butanetricarboxylic acid,

disodium (3RS,4RS)-3-carboxylate-4-hydroxy-4-[N-[(1RS,2RS)-1-methyl-2-(4-nitrophenyl)-3-(5- (phenylcarbamoyl)-2-furyl)propyl]-N-(2-naphthylmethyl)carbamoyl]butanoate,

disodium (3RS,4RS)-3-carboxylate-4-hydroxy-4-[N-[(1RS,2RS)-1-methyl-2-(4-nitrophenyl)-3-(5- (phenylcarbamoyl)-2-furyl)propyl]-N-(2-naphthylmethyl)carbamoyl]butanoate,

3-tert-butoxycarbonyl-4-hydroxy-4-[N-[(1RS,2RS)-1-methyl-2-(4-nitrophenyl)-3-(5- (phenylcarbamoyl)-2-fI )propyl]-N-(2-naphthylmethyl)carbamoyl]-3-butenoic acid,

3-tert-butoxycarbonyl-4-hydroxy-4-[N-[(1RS,2RS)-1-methyl-2-(3,4-methylenedioxyphenyl)-3-(5- (phenylcarbanoyl)-2-furyl)propyl]-N-(2-naphthylmethyl)carbamoyl]-3-butenoic acid,

3-tert-butoxycarbonyl-4-hydroxy-4-[-((1RS,2RS)-1-methyl-2-(4-nitrophenyl)-3-(3-phenoxymethylphenyl)propyl)-N-(2-Naphthylmethyl)carbamoyl]-3-butenoic acid,

4-hydroxy-3-methoxycarbonyl-4-[N-[(1RS,2RS)-1-methyl-2-(3,4-methylenedioxyphenyl)-3- (S-(phenylcarbamoyl)-2-I)propyl]-N-(2-naphthylmethyl)carbamoyl]-3-butenoic acid,

3-allyloxycarbonyl-4-hydroxy-4-[N-[(1RS,2RS)-1-methyl-2-(3,4-methylenedioxyphenyl)-3-(5- (phenylcarbamoyl)-2-furyl)propyl]-N-(2-naphthylmethyl)carbamoyl]-3-butenoic acid,

5-hydroxy-4-isopropylcarbonyl-5-[N-[(1RS,2RS)-1-methyl-2-(4-nitrophenyl)-3-(5- (phenylcarbamoyl)-2-ftiryl)propyl]-N-(2-naphthylmethyl)carbamoyl]-4-pentenoic acid,

3-tert-butoxycarbonyl-4- (N-(2,3-dichlorobenzyl)-N-[(1RS,2RS)-1-methyl-2-(4-nitrophenyl)-3-(5- (phenylcarbanoyl)-2-furyl)propyl]carbamoyl]-4-hydroxy-3-butenoic acid,or

a pharmaceutically acceptable salt or optical isomer thereof. A further embodiment of the specific farnesyl pyrophosphate-competitive inhibitors includes:

disodium (3RS.4RS)-4-[N-1(1RS,2RS,4E)-5-(2-benzoxazolyl)-1-methyl-2-(3,4-methylenedioxyphenyl)-4-pentenyl)-N-(2-naphthylmethyl)carbamoyl]-3-carboxyl-4-hyroxybutanoate

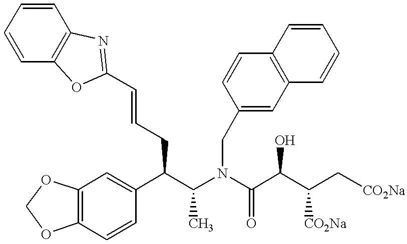

and sodium 4-[N-1(1R,2R,4E)-5-(2-benzoxazolyl)-1-methyl-2-(3,4-methylenedioxyphenyl)-4-pentenyl)-N-(2-naphthylmethyl)carbamoyl]-3-tert-butoxycarbonyl-4-hydroxy-3-butenoate

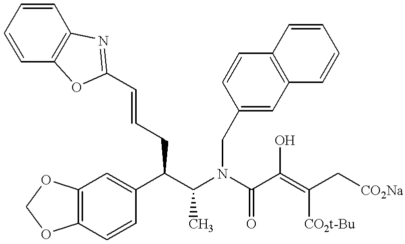

Other farnesyl-protein transferase inhibitor compounds that are suitable for use in the methods of the invention are disclosed in the following publications: European Patent Publication Nos. 0 537 008; and 0 540 782; PCT Patent Publication Nos. WO 94/1935; WO 95/12572; and WO 95/08546.

The inhibitor compounds suitable for use in the methods of the invention may have asymmetric centers and occur as racemates,racemic mixtures,and as individual diastereomers,with all possible isomers,including optical isomers,being included in the present invention. Unless otherwise specified,named amino acids are understood to have the natural “L” stereoconfiguration. Further,inhibitor compounds suitable for use in the methods of the invention may have enol form and keto form tautomers, depending upon the form of its substituents. The compounds of the present invention includes such enol form and keto form isomers and their mixtures. Additionally, when a hydroxyl group is present at the γ or δ-position of the terminal carboxyl group or of a carboxyl group when such a carboxyl group or a lower carboxyalkyl group is present on the saturated or unsaturated aliphatic hydrocarbon group represented by A in the formulas (f

f) and (gg),such a hydroxyl group and a carboxyl group may form an intramolecular ester i.e. a 5-membered or 6-membered lactone ring.

The following definitions apply to the above-discussed compounds,including those of the above general formulae (a) through (ee):

“Alkyl” is intended to include both branched and straight-chain saturated aliphatic hydrocarbon groups having the specified number of carbon atoms. “Cycloalkyl” is intended to include non-aromatic cyclic hydrocarbon groups having the specified number of carbon atoms. Examples of cycloalkyl groups include cyclopropyl, cyclobutyl, cyclopentyl, cyclohexyl and the like. “Alkenyl” groups include those groups having the specified number of carbon atoms and having one or several double bonds. Examples of alkenyl groups include vinyl, allyl, isopropenyl, pentenyl, hexenyl, heptenyl, cyclopropenyl, cyclobutenyl, cyclopentenyl, cyclohexenyl, l-propenyl, 2-butenyl, 2-methyl-2-butenyl, isoprenyl, farnesyl, geranyl, geranylgeranyl and the like. As used herein, “aryl” is intended to include any stable monocyclic, bicyclic or tricyclic carbon ring(s) of up to 7 members in each ring, wherein at least one ring is aromatic. Examples of aryl groups include phenyl, naphthyl, anthracenyl, biphenyl, tetrahydronaphthyl, indanyl, phenanthrenyl and the like. The term heterocycle or heterocyclic, as used herein, represents a stable 5 to 7 membered monocyclic or stable 8 to 11 membered bicyclic or stable I 1-membered tricyclic heterocycle ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothio-pyranyl sulfone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, 2-oxopiperazinyl, 2-oxopiperidinyl, 2-oxopyrrolidinyl, piperidyl, piperazinyl, pyridyl, pyridyl N-oxide, pyridonyl, pyrazinyl, pyrazolidinyl, pyrazolyl, pyrimidinyl, pyrrolidinyl, pyrrolyl, quinazolinyl, quinolinyl, quinolinyl N-oxide, quinoxalinyl, tetrahydrofuryl, tetrahydroisoquinolinyl, tetrahydro-quinolinyl, thiamorpholinyl, thiamorpholinyl sulfoxide, thiazolyl, thiazolinyl, thienofuyl, thienothienyl, and thienyl.

As used herein, the terms “substituted aryl”, “substituted heterocycle” and “substituted cycloalkyl” are intended to include the cyclic group which is substituted with 1 or 2 substituents selected from the group which includes but is not limited to F, Cl, Br, CF₃, NH2, N(C₁-C₆ alkyl)₂, NO₂, CN, (C₁-C₆ alkyl)O—, —OH, (C₁-C₆ alkyl)S(O)ₘ—, (C₁-C₆ alkyl)C(O)NH—, H₂N—C(NH)—, (C₁-C₆ alkyl)C(O)-, (C₁-C₆ alkyl)OC(O)—, N₃, (C₁-C₆ alkyl)OC(O)NR- and C₁-C₂₀ alkyl.

The following structure:

[chemical structure]

represents a cyclic amine moiety having 5 or 6 members in the ring, such a cyclic amine which may be optionally fused to a phenyl or cyclohexyl ring. Examples of such a cyclic amine moiety include, but are not limited to, the following specific structures:

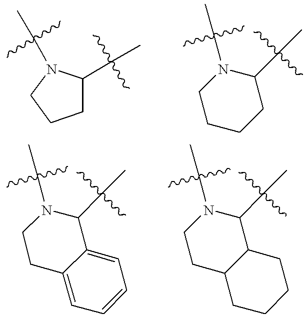

It is also understood that substitution on the cyclic amine moiety by R^{2a}, R²b, R^{1a} and R^{1b} may be on different carbon atoms or on the same carbon atom.

When R^{2a} and R²b, and R³ and R⁴ are combined to form (CH₂O)ₘ—, cyclic moieties are formed. Examples of such cyclic moieties include, but are not limited to:

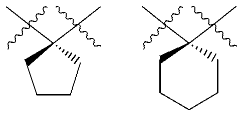

When R^{5a} and R^{5b} are combined to form —(CH₂)ₛ—, cyclic hereinabove for R³ and R⁴ are formed. In addition, such cyclic moieties may optionally include a heteroatom(s). Examples of such heteroatom-containing cyclic moieties include, but are not limited to:

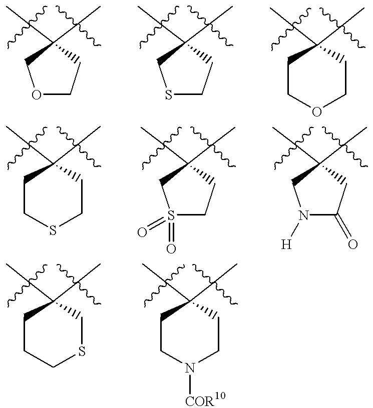

As used herein, the phrase “nitrogen containing C₄-C₉ mono or bicyclic ring system wherein the non-nitrogen containing ring may be a C₆ aromatic ring, a C₅-C₇ saturated ring or a heterocycle” which defines moiety “Q” includes but is not limited to the following ring systems:

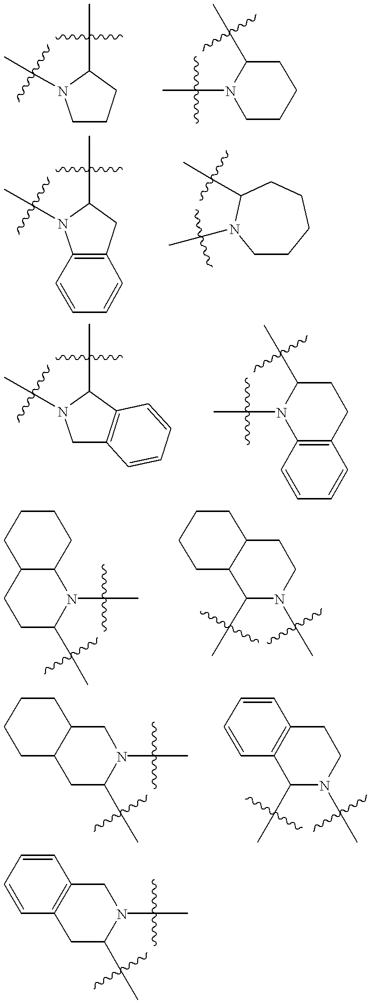

It is intended that the definition of any substituent or variable (e.g., R¹⁰, Z, n, etc.) at a particular location in a molecule be independent of its definitions elsewhere in that molecule. Thus, N(R¹⁰)₂ represents—NHH, —NHCH₃, —NHC₂H₅, etc. It is understood that substituents and substitution patterns on a particular inhibitor compounds suitable for use in the methods of the invention can be selected by one of ordinary skill in the art to provide compounds that are chemically stable and that can be readily synthesized by known techniques.

The pharmaceutically acceptable salts of inhibitor compounds for use in the methods of the invention include known non-toxic salts, e.g. pharmaceutically acceptable inorganic or organic acids such as the following acids: hydrochloric, hydrobromic, sulfuric, sulfamic, phosphoric, nitric, acetic, propionic, succinic, glycolic, stearic, lactic, malic, tartaric, citric, ascorbic, pamoic, maleic, hydroxymaleic, phenyl-acetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. The pharmaceutically acceptable salts of inhibitor compounds for use in the methods of the invention can be synthesized from the corresponding inhibitor of this invention which contain a basic moiety by conventional chemical methods. Generally, the salts are prepared by reacting the free base with stoichiometric amounts or with an excess of the .desired salt-forming inorganic or organic acid in a suitable solvent or various combinations of solvents.

The following definitions apply to compounds of the above general formulae (ff) through (gg): The aryl group means a phenyl group, a naphthyl group or an anthryl group. A phenyl group or a naphthyl group is preferred.

The heteroaromatic ring group means a 5-membered or 6-membered monocyclic aromatic heterocyclic group containing one or two heteroatoms, which are the same or different, selected from the group consisting of an oxygen atom, a nitrogen atom and a sulfur atom, or a fused aromatic heterocyclic group having such a monocyclic aromatic heterocyclic group fused with the above-mentioned aryl group or having the same or different such monocyclic aromatic heterocyclic groups fused with each other, which may, for example, be a pyrrolyl group, an imidazolyl group, a pyrazolyl group, a pyridyl group, a pyrazinyl group, a pyrimidinyl group, a pyridazinyl group, an oxazolyl group, an isoxazolyl group, a furyl group, a thienyl group, a thiazolyl group, an isothiazolyl group, an indolyl group, a benzofuranyl group, a benzothienyl group, a benzimidazolyl group, a benzoxazolyl group, a benzisoxazolyl group, a benzothiazolyl group, a benzisothiazolyl group, an indazolyl group, a purinyl group, a quinolyl group, an isoquinolyl group, a phthalazinyl group, a naphthylidinyl group, a quinoxalinyl group, a quinazolinyl group, a cinnolinyl group or a pteridinyl group. Among them, a furyl group, a thienyl group, a pyridyl group, a pyrimidinyl group, an oxazolyl group, an isoxazolyl group, a thiazolyl group, a benzofuranyl group, a benzothienyl group, a benzimidazolyl group, a benzoxazolyl group, a benzothiazolyl group or a quinolyl group is preferred. The lower alkyl group means a C₁₋₆ linear or branched alkyl group, which may, for example, be a methyl group, an ethyl group, a propyl group, an isopropyl group, a butyl group, a sec-butyl group, a tert-butyl group, a pentyl group or a hexyl group. Among them, a methyl group or an ethyl group is preferred. The lower hydroxyalkyl group means the above-mentioned lower alkyl group having a hydroxyl group, i.e. a C₁₋₆ hydroxyalkyl group, such as a hydroxymethyl group, a hydroxyethyl group, a hydroxypropyl group or a hydroxybutyl group. Among them, a hydroxymethyl group or a hydroxyethyl group is preferred. The lower alkoxy group means a C₁₋₆ alkoxy or alkylenedioxy group, which may, for example, be a methoxy group, an ethoxy group, a propoxy group, an isopropoxy group, a butoxy group, a tert-butoxy group, a methylenedioxy group, an ethylenedioxy group or a trimethylenedioxy group. Among them, a methoxy group, an ethoxy group or a methylenedioxy group is preferred. The lower carboxyalkyl group means the above-mentioned lower alkyl group having a carboxyl group, i.e. a C₁₋₇ carboxyalkyl group, such as a carboxymethyl group, a carboxyethyl group, a carboxypropyl group or a carboxybutyl group. Among them, a carboxymethyl group or a carboxyethyl group is preferred. The aralkyl group means the above-mentioned lower alkyl group having the above-mentioned aryl group, such as a benzyl group, a phenethyl group, a 3-phenylpropyl group, a 1-naphthylmethyl group, a 2-naphthylmethyl group or a 1-(2-naphthyl)ethyl group. Among them, a benzyl group, a phenethyl group or a 2-naphthylmethyl group is preferred. The saturated aliphatic hydrocarbon group may, for example, be an ethylene group, a trimethylene group, a tetramethylene group, a pentamethylene group, a hexamethylene group, a heptamethylene group or an octamethylene group. For example, a trimethylene group, a tetramethylene group or a pentamethylene group is preferred.

The unsaturated aliphatic hydrocarbon group means an unsaturated aliphatic hydrocarbon group having one or more, preferably one or two double bonds, at optional position(s) on the carbon chain, which may, for example, be a vinylene group, a propenylene group, a 1-butenylene group, a 2-butenylene group, a 1,3-butadienylene group, a 1-pentenylene group, a 2-pentenylene group, a 1,3-pentadienylene group, a 1,4-pentadienylene group, a 1-hexenylene group, a 2-hexenylene group, a 3-hexenylene group, a 1,3-hexadienylene group, a 1,4-hexadienylene group, a 1,5-hexadienylene group, a 1,3,5-hexatrienylene group, a 1-heptenylene group, a 2-heptenylene group, a 3-heptenylene group, a 1,3-heptadienylene group, a 1,4-heptadienylene group, a 1,5-heptadienylene group, a 1,6-heptadienylene group, a 1,3,5-heptatrienylene group, a 1-octenylene group, a 2-octenylene group, a 3-octenylene group, a 4-octenylene group, a 1,3-octadienylene group, a 1,4-octadienylene group, a 1,5-octadienylene group, a 1,6-octadienylene group, a 1,7-octadienylene group, a 2, 4-octadienylene group, a 2, 5-octadienylene group, a 2, 6-octadienylene group, a 3,5-octadienylene group, a 1,3,5-octatrienylene group, a 2, 4, 6-octatrienylene group or a 1,3,5,7-octatetraenylene group. Among them, a propenylene group, a I-butenylene group, a 1,3-butadienylene group or a 1-pentenylene group is preferred. The halogen atom may be a fluorine atom, a chlorine atom, a bromine atom or an iodine atom. For example, a fluorine atom or a chlorine atom is preferred.

The lower alkoxycarbonyl group means a C₁₋₇ alkoxycarbonyl group, such as a methoxycarbonyl group, an ethoxycarbonyl group, a propoxycarbonyl group, a butoxycarbonyl group or a tert-butoxycarbonyl group. Among them, a methoxycarbonyl group or an ethoxycarbonyl group is preferred. The lower alkylcarbamoyl group means a carbamoyl group mono-substituted or di-substituted by the above-mentioned lower alkyl group, such as a methylcarbamoyl group, an ethylcarbamoyl group, a dimethylcarbamoyl group or a diethylcarbamoyl group. The lower fluoroalkyl group means the above-mentioned lower alkyl group having fluorine atom(s), i.e. a C₁₋₆ fluoroalkyl group, such as a fluoromethyl group, a difluoromethyl group, a trifluoromethyl group, a l-fluoroethyl group, a 2-fluoroethyl group, a 2,2,2-trifluoroethyl group or a pentafluoroethyl group.

The salt of the compound of a formula (ff) or (gg) may be a pharmaceutically acceptable common salt, which may, for example, be a base-addition salt of the terminal carboxyl group or of a carboxyl group when R⁴ and/or R⁵ or R³ and/or R⁴ is a carboxyl group, or when a carboxyl group or a lower carboxyalkyl group is present on a saturated or unsaturated aliphatic hydrocarbon group represented by A in the formulas (ff) and (gg), or an acid-addition salt of an amino group when R⁴ and/or R⁵ or R³ and/or R⁴ is an amino group, or of a basic heteroaromatic ring when such a basic heteroaromatic ring is present.

The base-addition salt may, for example, be an alkali metal salt such as a sodium salt or a potassium salt; an alkaline earth metal salt such as a calcium salt or a magnesium salt; an ammonium salt; or an organic amine salt such as a trimethylamine salt, a triethylamine salt, a dicyclohexylamine salt, an ethanolamine salt, a diethanolamine salt, a triethanolamine salt, a procaine salt or an N,N′-dibenzylethylenediamine salt. The acid-addition salt may, for example, be an inorganic acid salt such as a hydrochloride, a sulfate, a nitrate, a phosphate or a perchlorate; an organic acid salt such as a maleate, a fumarate, a tartrate, a citrate, an ascorbate or a trifluoroacetate; or a sulfonic acid salt such as a methanesulfonate, an isethionate, a benzenesulfonate or a p-toluenesulfonate.

The ester of a compound of the formula (ff) or (gg) means a pharmaceutically acceptable common ester of the terminal carboxyl group or of a carboxyl group when R⁴ and/or Rs or R³ and/or R⁴ is a carboxyl group, or when a carboxyl group or a lower carboxyalkyl group is present on the saturated or unsaturated aliphatic hydrocarbon group represented by A in the formulas (ff) and (gg). It may, for example, be an ester with a lower alkyl group such as a methyl group, an ethyl group, a propyl group, an isopropyl group, a butyl group, a sec-butyl group, a tert-butyl group, a cyclopropyl group or a cyclopentyl group, an ester with an aralkyl group such as a benzyl group or a phenethyl group, an ester with a lower alkenyl group such as an allyl group or a 2-butenyl group, an ester with a lower alkoxyalkyl group such as a methoxymethyl group, a 2-methoxyethyl group or a 2-ethoxyethyl group, an ester with a lower alkanoyloxyalkyl group such as an acetoxymethyl group, a pivaloyloxymethyl group or a pivaloyloxyethyl group, an ester with a lower alkoxycarbonylalkyl group such as a methoxycarbonylmethyl group or an isopropoxycarbonylmethyl group, an ester with a lower carboxyalkyl group such as a carboxymethyl group, an ester with a lower alkoxycarbonyloxyalkyl group such as a I -(ethoxycarbonyloxy) ethyl group or a 1-(cyclohexyloxycarbonyloxy)ethyl group, an ester with an lower carbarnoyloxyalkyl group such as a carbamoyloxymethyl group, an ester with a phthalidyl group, or an ester with a (5-substituted-2-oxo-1,3-dioxol-4-yl)methyl group such as a (5-methyl-2-oxo-1,3-dioxol-4yl)methyl group.

At least some of the inhibitor compounds useful in the methods of the invention can be synthesized from their constituent amino acids by conventional peptide synthesis techniques, and the additional methods described below. Standard methods of peptide synthesis are disclosed, for example, in the following works: Schroeder et al., The Peptides, Vol. 1, Academic Press 1965, or Bodanszky et al., Peptide Synthesis, Interscience Publishers, 1966, or McOmie (ed.) “Protective Groups in Organic Chemistry”, Plenum Press, 1973, or Barany et al., “The Peptides: Analysis, Synthesis, Biology” 2, Chapter 1, Academic Press, 1980, or Stewart et al., “Solid Phase Peptide Synthesis”, Second Edition, Pierce Chemical Company, 1984. Also useful in exemplifying syntheses of specific unnatural amino acid residues are European Patent Application No. 0 350 163 A2 (particularly page 51-52) and J. E. Baldwin et al., Tetrahedron, 50:5049-5066 (1994). With regards to the synthesis of the above discussed compounds containing a (β-acetylamino)alanine residue at the C-terminus, use of the commercially available N-Z-L-2,3-diaminopropionic acid (Fluk

a) as a starting material is preferred. In general, methods for preparation of the above discussed compounds are known in the art and disclosed e.g. in the above-mentioned publications. Detailed synthetic procedures are also disclosed in PCT/US96/11022. Useful FTase inhibitor compounds are also commercially available.

In the methods of the invention, an FTase inhibitor compound may be administered to a subject by a variety of routes including parenteral (including subcutaneous, intramuscular and intradermal), topical (including eye drops, buccal, sublingual) oral, nasal and the like. Intraviteal or periocular injection of a compound may be a preferred administration route to provide more direct treatment.

Inhibitor compounds for use in the methods of the invention can be employed, either alone or in combination with one or more other therapeutic agents, as a pharmaceutical composition in mixture with conventional excipient, i.e., pharmaceutically acceptable organic or inorganic carrier substances suitable for a desired route of administration which do not deleteriously react with the active compounds and are not deleterious to the recipient thereof. Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds.

For parenteral application, particularly suitable are solutions, preferably oily or aqueous solutions as well as suspensions, emulsions, or implants, including suppositories. Ampules are convenient unit dosages.

For enteral application, particularly suitable are tablets, dragees or capsules having talc and/or carbohydrate carrier binder or the like, the carrier preferably being lactose and/or corn starch and/or potato starch. A syrup, elixir or the like can be used wherein a sweetened vehicle is employed. Sustained release compositions can be formulated including those wherein the active component is protected with differentially degradable coatings, e.g., by microencapsulation, multiple coatings, etc.

It will be appreciated that the actual preferred amounts of active compounds used in a given therapy will vary according to the specific compound being utilized, the particular compositions formulated, the mode of application, the particular site of administration, etc. Optimal administration rates for a given protocol of administration can be readily ascertained by those skilled in the art using conventional dosage determination tests conducted with regard to the foregoing guidelines.

All documents mentioned herein are incorporated herein by reference.

This invention has been described in detail with reference to preferred embodiments thereof. However, it will be appreciated that those skilled in the art, upon consideration of this disclosure, may make modifications and improvements within the spirit and scope of the invention.

What is claimed is:

1. A method for treating ocular neovascularization in a mamrmal suffering from or susceptible to ocular neovascularization, comprising administering to the mammal a therapeutically effective amount of a compound that inhibits famesyl-protein transferase.

2. The method of claim 1 wherein the mammal is suffering from or susceptible to a vasculopathy that affects retinal or chorodial vessels.

3. The method of claim 1 wherein the mammal is suffering from or susceptible to diabetic retinopathy, retinopathy of prematurity, retinal vein or artery occulsion, age-related macular degeneration, corneal graft rejection, neovascular glaucoma, or Eales disease.

4. A method for treating a mammal suffering from or susceptible to a vasculopathy that affects retinal vessels, comprising administering to the mammal a therapeutically effective amount of a compound that inhibits famesyl-protein transferase.

5. A method for treating a disorder that is diabetic retinopathy, retinopathy of prematurity, retinal vein or artery occulsion, age-related macular degeneration, corneal graft rejection, neovascular glaucoma, or Eales disease, comprising administering to a mammal suffering from or susceptible to the disorder a therapeutically effective amount of a compound that inhibits famesyl-protein transferase.

6. A method of any one of claims 1 through 5 wherein the farnesyl-protein transferase inhibitor compound has an IC₅₀ of about 100 nM or less in a standard in vitro famesyl-protein transferase inhibition assay.

7. A method of any one of claims 1 through 5 wherein the famesyl-protein transferase inhibitor compound has an IC₅₀ of about 50 nM or less in a standard in vitro famesyl-protein transferase inhibition assay.

8. A method of any one of claims 1 through 7 wherein the famesyl-protein inhibitor compound exhibits at least about a 20-fold greater inhibition of farnesyl-protein transferase relative to inhibition of geranylgeranyltransferase-protein transferase I as measured in standard in vitro farnesyl-protein transferase and geranylgeranyl-protein tansferase I inhibition assays.

9. A method of claim 6 or 7 wherein the farnesyl-protein transferase inhibitor compound has an IC₅₀ of about 500 nM or less in a standard in vitro geranylgeranyl-protein tansferase I inhibition assay.

10. A method of any one of claims 1 through 9 wherein the farnesyl-protein transferase inhibitor compound is of any one of general groups (a) through (gg) inclusive as those are groups set forth above.

11. A method of any one of claims 1 through 5 wherein the farnesyl-protein transferase inhibitor compound is: 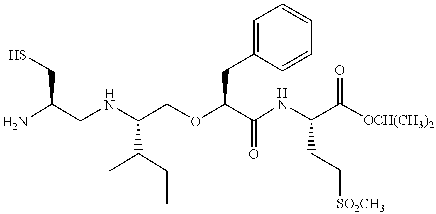
or a pharmaceutically acceptable salt thereof.

12. The method of any one of claims 1 through 5 wherein the farnesyl-protein transferase inhibitor curnpound is: 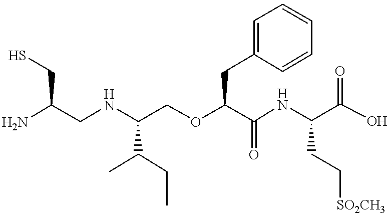
or a pharmaceutically acceptable salt thereof.

13. The method of any one of claims 1 through 5 wherein the farnesyl-protein transferase inhibitor compound is: 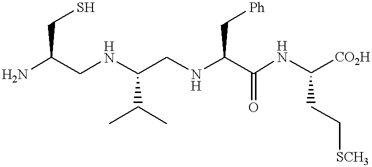
or a pharmaceutically acceptable salt thereof.

14. The method of any one of claims 1 through 5 wherein the compound is 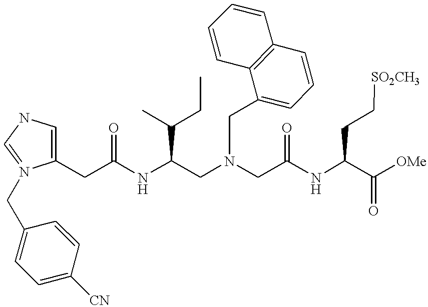
or a pharmaceutically acceptable salt thereof.
